(19) United States
(12) Patent Application Publication
(10) Pub. No.: US 2020/0390909 A1
(43) Pub. Date: Dec. 17, 2020

(54) DRUG FRAGMENT IMAGING AGENT CONJUGATES

(71) Applicant: The Board of Trustees of the Leland Stanford Junior University, Stanford, CA (US)

(72) Inventors: Stefan Harmsen, Menlo Park, CA (US); Stephan Rogalla, Stanford, CA (US); Sanjiv Gambhir, Portola Valley, CA (US)

(21) Appl. No.: 16/977,720

(22) PCT Filed: Mar. 6, 2019
(86) PCT No.: PCT/US2019/020966

§ 371 Date: Sep. 2, 2020

Related U.S. Application Data
(60) Provisional application No. 62/639,795, filed on Mar. 7, 2018.
(60) Provisional application No. 62/798,167, filed on Jan. 29, 2019.

(51) Int. Cl. A61K 49/00; A61K 31/506; A61K 31/519; A61K 31/5375; C09B 23/04; A61K 47/10; A61K 47/02

(52) U.S. Cl. CPC A61K 49/0032; A61K 31/506; A61K 31/519; A61K 9/0053; C09B 23/04; A61K 47/10; A61K 47/02; A61K 31/5375

(57) ABSTRACT

Functional dyes and methods of use are provided. The dyes are useful in a variety of medical applications including, but not limited to, diagnostic imaging and therapy, endoscopic applications for the detection of tumors and other abnormalities, particularly with oral administration of the dyes.

### CROSS-REFERENCE

This application claims the benefit of U.S. Provisional Patent Application No. 62/639,795, filed Mar. 7, 2018 and U.S. Provisional Patent Application No. 62/798,167, filed Jan. 29, 2019, which applications are incorporated herein by reference in their entirety.

### FEDERALLY SPONSORED RESEARCH AND DEVELOPMENT

This invention was made with Government support under contract CA182043 awarded by the National Institutes of Health. The Government has certain rights in the invention.

### BACKGROUND

Non-invasive, optical imaging methods offer a number of advantages over other imaging methods: they provide generally high sensitivity, do not require exposure of test subjects or lab personnel to ionizing radiation, can allow for simultaneous use of multiple, distinguishable probes (important in molecular imaging), and offer high temporal and spatial resolution (important in functional imaging and in vivo microscopy, respectively).

In high-risk patients who already undergo periodic white light endoscopic surveillance, it is estimated that about three times more dysplastic lesions, the most clinically relevant marker for malignant progression, are missed relative to healthy individuals. Important reasons for this high miss rate are the subtle appearance of such lesions, often on a background of inflammation, and sampling errors inherent to a random-biopsy surveillance paradigm. Further, endoscopic assessment and diagnosis of GI tract lesions is operator-dependent and prone to subjectivity, which increases inter-observer variability and thus further compromises diagnostic accuracy. Lastly, surveillance for gastric and colorectal lesions can be challenging due to the large surface area that needs to be surveyed, poor preparation and lack of time for the procedure. Moreover, even when disease is detected, it is often difficult to determine the true extent of the lesion, thus hampering the ability to achieve complete minimally invasive (endoscopic) therapeutic intervention through resection or ablation.

Consequently, approximately 33% of GI tract lesions recur at or near the therapeutic site, commonly requiring more aggressive yet often non-curative (systemic) treatments that also negatively impact the patients' quality of life. There is an unmet need for novel imaging approaches that reliably enable highly sensitive detection of cancerous or precancerous lesions.

### SUMMARY

Functional dyes are provided that highlight cancerous or precancerous lesions following intravenous, oral or topical administration. In some embodiments, the functional dye has the formula:

D-L-F

wherein:
D is a near infrared fluorescent dye;
L is an optional linker; and
F is a small molecule that targets a protein.

In some embodiments a pharmaceutical composition is provided, comprising one or more of the functional dyes described here, and a pharmaceutically acceptable excipient. In some embodiments the pharmaceutical composition is provided in a unit dose. In some embodiments the pharmaceutical composition is provided for oral administration.

In some embodiments a method is provided for imaging cancerous or precancerous lesions, including without limitation dysplastic lesions of the gastrointestinal tract, breast, brain, prostate, pancreas, skin, bladder, head, neck and thyroid, the method comprising administering an effective dose of a functional dye described herein. In some embodiments the functional dye is administered orally. In some embodiments the functional dye is sprayed onto regions for analysis. It is shown herein that the dye is selectively retained by malignant and premalignant lesions, allowing detection of such lesions. The presence, absence, distribution, or level of optical signal emitted by the functional dye is indicative of a disease state.

In some embodiments detection of the dye is performed using fluorescence endoscopy. In some embodiments, visualization of a lesion is used to guide a biopsy or surgery. Fluorescence guidance during endoscopy improves diagnostic accuracy and/or therapeutic efficacy.

Also provided herein is a method of in vivo optical imaging, the method comprising (a) administering to a subject a functional dye of the present invention; (b) allowing time for the dye to distribute within the subject or to contact or interact with a biological target; (c) illuminating the subject with light of a wavelength absorbable by the functional dye; and (d) detecting the optical signal emitted by the functional dye. The optical signal generated by the dyes of the invention, whether collected by tomographic, reflectance, planar, endoscopic, microscopic, surgical goggles, video imaging technologies, capsule endoscopy, or other methods such as microscopy including intravital and two-photon microscopy, and whether used quantitatively or qualitatively, is also considered to be an aspect of the invention.

The imaging method steps can also be repeated at predetermined intervals thereby allowing for the evaluation of emitted signal containing functional dye in a subject or sample over time. The emitted signal may take the form of an image. The subject may be a vertebrate animal, for example, a mammal, including a human.

In some embodiments, the functional dyes are used to provide positive contrast-enhancement of premalignant or malignant lesions during endoscopic examination of the GI tract, such as mouth, throat, esophagus, stomach, duodenum, ileum, colon, rectum and pancreas. In this way, the functional dyes enable fluorescent-guided biopsy or fluorescent-guided therapy in patients that are at increased risk of developing such lesions (e.g. patients with Barrett's esophagus, familial adenomatous polyposis (FAP) patients, etc.). In some embodiments, the functional dyes are used to provide positive contrast enhancement of a cancer tumor, e.g. a breast cancer tumor, and can be used during image-guided resection of tumor tissue.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1A-1B. Sensitive detection of intestinal adenomas after oral administration of exemplary functional dye (Compound (1), SU-783) in preclinical CRC model. FIG. 1A Wide-field near-infrared fluorescence imaging (I_{ex,em}=785, >800 nm) of intestinal section of an ApcMin/+ mouse 5 h after oral administration of SU-783 at a dose of 0.4 mg/kg (in 8.4% bicarbonate). Lesions 1 (TBR=6.1), 2 (TBR=5.3), and 3 (TBR=5.2) were sectioned and processed for histopathological examination by a veterinary pathologist. FIG. 1B All NIRF-positive lesions were identified as sessile adenomas (arrow heads). The sensitivity and specificity is particularly well-illustrated by tissue section 3, where SU-783 discretely highlights the sessile adenoma (high fluorescence signal) without staining normal mucosa. Of note, no SU-783-associated fluorescence signal was found in the blood, liver, kidneys, or bladder indicating that SU-783 was not systemically absorbed following oral administration.

FIG. 2. Illustrates the synthesis of exemplary functional Dye SU-783 (Compound (1)) from the commercially available dye IR-783.

FIG. 3A-3C. Functional dye stability. FIG. 3A Fluorescence intensity of exemplary functional dyes derived from near infrared fluorescent dyes IR783, S0456 and S2180, compared to indocyanine green (ICG), in water imaged each day for a week. FIG. 3B Actual NIRF image of aqueous solutions of the functional dyes at day 0 and day 6. FIG. 3C NIR-II fluorescence intensity of S0456 and S2180 dyes after 2 weeks in water compared to a freshly prepared solution of ICG in water.

FIG. 4. Illustrates an NIRF endoscopy of Apc^{Pirc/+} rats following oral administration of an exemplary functional dye (Compound (1)). An apparent adenoma is detected both by the white-light endoscope (left panel) and highlighted by Compound (1) using NIRF endoscopy (right panel).

FIG. 5. Illustrates that at exemplary functional dye (Compound (1)) highlights human adenomas.

FIG. 6A-6D. Highlighting a breast tumor following oral administration of an exemplary systemic functional dye (Compound (8), SU780). FIG. 6A NIRF imaging of the intact mouse 24 h post oral gavage of SU780. FIG. 6B NIRF imaging of resected tumor demonstrates that SU780 highlights the tumor following oral administration. FIG. 6C H&E stained tumor section of the tumor demonstrated this was a breast cancer. FIG. 6D Higher magnification of tumor margin (black square in C) demonstrates that SU780 highlights the finger-like infiltration of the breast cancer cells into normal mammary fat pad (arrow heads) following oral administration of the dye.

### DESCRIPTION OF THE EMBODIMENTS

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

### Definitions

Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

By the term “imaging agent” is meant a compound suitable for optical imaging of a region of interest of the whole (i.e. intact) mammalian body in vivo. Preferably, the mammal is a human subject. The imaging may be invasive (e.g. intra-operative or endoscopic) or non-invasive. The imaging may optionally be used to facilitate biopsy (e.g. via a biopsy channel in an endoscope instrument), or tumor resection (e.g. during intra-operative procedures via tumor margin identification).

Where compounds described herein contain one or more chiral centers and/or double-bond isomers (i.e., geometric isomers), enantiomers or diastereomers, all possible enantiomers and stereoisomers of the compounds including the stereoisomerically pure form (e.g., geometrically pure, enantiomerically pure or diastereomerically pure) and enantiomerc and stereoisomeric mixtures are included in the description of the compounds herein. Enantiomeric and stereoisomeric mixtures can be resolved into their component enantiomers or stereoisomers using separation techniques or chiral synthesis techniques well known to the skilled artisan. The compounds can also exist in several tautomeric forms including the enol form, the keto form and mixtures thereof. Accordingly, the chemical structures depicted herein encompass all possible tautomeric forms of the illustrated compounds. The compounds described also include isotopically labeled compounds where one or more atoms have an atomic mass different from the atomic mass conventionally found in nature. Examples of isotopes that can be incorporated into the compounds disclosed herein include, but are not limited to, ²H, ³H, ¹¹C, ¹³C, ¹⁴C, ¹⁵N, ¹⁸O, ¹⁷O, etc. Compounds can exist in unsolvated forms as well as solvated forms, including hydrated forms. In general, compounds can be hydrated or solvated. Certain compounds can exist in multiple crystalline or amorphous forms. In general, all physical forms are equivalent for the uses contemplated herein and are intended to be within the scope of the present disclosure.

The term “heteroatom” as used herein refers to an atom that is not carbon or hydrogen, such as O, S, N, Se or Te. In certain cases, the heteroatom may be a cationic heteroatom, such as N⁺, O⁺, S⁺, Se⁺, Te⁺.

The term “alkyl” as used herein refers to a branched or unbranched saturated hydrocarbon group (i.e., a mono-radical) typically although not necessarily containing 1 to about 24 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl and the like. Generally, although not necessarily, alkyl groups herein may contain 1 to about 18 carbon atoms. The term “lower alkyl” intends an alkyl group of 1 to 6 carbon atoms. “Substituted alkyl” refers to alkyl substituted with one or more substituent groups, and this includes instances wherein two hydrogen atoms from the same carbon atom in an alkyl substituent are replaced, such as in a carbonyl group (i.e., a substituted alkyl group may include a —C(═O)— moiety). The terms “heteroatom-containing alkyl” and “heteroalkyl” refer to an alkyl substituent in which at least one carbon atom is replaced with a heteroatom, as described in further detail infra. If not otherwise indicated, the terms “alkyl” and “lower alkyl” include linear, branched, cyclic, bridged, unsubstituted, substituted, and/or heteroatom-containing alkyl or lower alkyl, respectively.

The term “substituted alkyl” refers to an alkyl group as defined herein wherein one or more carbon atoms in the alkyl chain have been optionally replaced with a heteroatom such as —O—, —N—, —S—, —S(O)ₙ— (where n is 0 to 2), —NR— (where R is hydrogen or alkyl) and having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl, —SO₂-heteroaryl, and —NR^{a}R^{b}, wherein R′ and R′ may be the same or different and are chosen from hydrogen, optionally substituted alkyl, cycloalkyl, alkenyl, cycloalkenyl, alkynyl, aryl, heteroaryl and heterocyclic.

The term “alkenyl” as used herein refers to a linear, branched or cyclic hydrocarbon group of 2 to about 24 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, and the like. Generally, although again not necessarily, alkenyl groups herein may contain 2 to about 18 carbon atoms, and for example may contain 2 to 12 carbon atoms. The term “lower alkenyl” intends an alkenyl group of 2 to 6 carbon atoms. The term “substituted alkenyl” refers to alkenyl substituted with one or more substituent groups, and the terms “heteroatom-containing alkenyl” and “heteroalkenyl” refer to alkenyl in which at least one carbon atom is replaced with a heteroatom. If not otherwise indicated, the terms “alkenyl” and “lower alkenyl” include linear, branched, cyclic, bridged, unsubstituted, substituted, and/or heteroatom-containing alkenyl and lower alkenyl, respectively.

“Substituted alkylene” refers to an alkylene group having from 1 to 3 hydrogens replaced with substituents as described for carbons in the definition of “substituted” below.

The term “alkynyl” as used herein refers to a linear or branched hydrocarbon group of 2 to 24 carbon atoms containing at least one triple bond, such as ethynyl, n-propynyl, and the like. Generally, although again not necessarily, alkynyl groups herein may contain 2 to about 18 carbon atoms, and such groups may further contain 2 to 12 carbon atoms. The term “lower alkynyl” intends an alkynyl group of 2 to 6 carbon atoms. The term “substituted alkynyl” refers to alkynyl substituted with one or more substituent groups, and the terms “heteroatom-containing alkynyl” and “heteroalkynyl” refer to alkynyl in which at least one carbon atom is replaced with a heteroatom. If not otherwise indicated, the terms “alkynyl” and “lower alkynyl” include linear, branched, unsubstituted, substituted, and/or heteroatom-containing alkynyl and lower alkynyl, respectively.

“Alkoxy” refers to the group —O-alkyl, wherein alkyl is as defined herein. Alkoxy includes, by way of example, methoxy, ethoxy, n-propoxy, isopropoxy, n-butoxy, t-butoxy, sec-butoxy, n-pentoxy, and the like. The term “alkoxy” also refers to the groups alkenyl-O—, cycloalkyl-O—, cycloalkenyl-O—, and alkynyl-O—, where alkenyl, cycloalkyl, cycloalkenyl, and alkynyl are as defined herein.

The term “substituted alkoxy” refers to the groups substituted alkyl-O—, substituted alkenyl-O—, substituted cycloalkyl-O—, substituted cycloalkenyl-O—, and substituted alkynyl-O— where substituted alkyl, substituted alkenyl, substituted cycloalkyl, substituted cycloalkenyl and substituted alkynyl are as defined herein.

The term “haloalkyl” refers to a substituted alkyl group as described above, wherein one or more hydrogen atoms on the alkyl group have been substituted with a halo group. Examples of such groups include, without limitation, fluoroalkyl groups, such as trifluoromethyl, difluoromethyl, trifluoroethyl and the like.

The term “alkylalkoxy” refers to the groups -alkylene-O-alkyl, alkylene-O-substituted alkyl, substituted alkylene-O-alkyl, and substituted alkylene-O-substituted alkyl wherein alkyl, substituted alkyl, alkylene and substituted alkylene are as defined herein.

“Alkenyl” refers to straight chain or branched hydrocarbyl groups having from 2 to 6 carbon atoms and preferably 2 to 4 carbon atoms and having at least 1 and preferably from 1 to 2 sites of double bond unsaturation. This term includes, by way of example, bi-vinyl, allyl, and but-3-en-1-yl. Included within this term are the cis and trans isomers or mixtures of these isomers.

The term “substituted alkenyl” refers to an alkenyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclyl-C(O)—, and substituted heterocyclyl-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. For example, acyl includes the “acetyl” group CH₃C(O)—.

“Acylamino” refers to the groups —NR²⁰C(O)alkyl, —NR²⁰C(O)substituted alkyl, N R²⁰C(O)cycloalkyl, —NR²⁰C(O)substituted cycloalkyl, —NR²⁰C(O)cycloalkenyl, —NR²⁰C(O)substituted cycloalkenyl, —NR^{Z}C(O)alkenyl, —NR²⁰C(O)substituted alkenyl, —NR²⁰C(O)alkynyl, —NR²⁰C(O)substituted alkynyl, —NR²⁰C(O)aryl, —NR²⁰C(O)substituted aryl, —NR²⁰C(O)heteroaryl, —NR²⁰C(O)substituted heteroaryl, —NR²⁰C(O)heterocyclic, and —NR²⁰C(O)substituted heterocyclic, wherein R²⁰ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyl” or the term “aminoacyl” refers to the group —C(O)NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonylamino” refers to the group —NR²¹C(O)NR²²R²³ where R²¹, R²², and R²³ are independently selected from hydrogen, alkyl, aryl or cycloalkyl, or where two R groups are joined to form a heterocyclyl group.

The term “alkoxycarbonylamino” refers to the group —NRC(O)OR where each R is independently hydrogen, alkyl, substituted alkyl, aryl, heteroaryl, or heterocyclyl wherein alkyl, substituted alkyl, aryl, heteroaryl, and heterocyclyl are as defined herein.

The term “acyloxy” refers to the groups alkyl-C(O)O—, substituted alkyl-C(O)O—, cycloalkyl-C(O)O—, substituted cycloalkyl-C(O)O—, aryl-C(O)O—, heteroaryl-C(O)O—, and heterocyclyl-C(O)O— wherein alkyl, substituted alkyl, cycloalkyl, substituted cycloalkyl, aryl, heteroaryl, and heterocyclyl are as defined herein.

The term “aryl” as used herein, and unless otherwise specified, refers to an aromatic substituent generally, although not necessarily, containing 5 to 30 carbon atoms and containing a single aromatic ring or multiple aromatic rings that are fused together, directly linked, or indirectly linked (such that the different aromatic rings are bound to a common group such as a methylene or ethylene moiety). Aryl groups may, for example, contain 5 to 20 carbon atoms, and as a further example, aryl groups may contain 5 to 12 carbon atoms. For example, aryl groups may contain one aromatic ring or two or more fused or linked aromatic rings (i.e., biaryl, aryl-substituted aryl, etc.). Examples include phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituent, in which at least one carbon atom is replaced with a heteroatom, as will be described in further detail infra. Aryl is intended to include stable cyclic, heterocyclic, polycyclic, and polyheterocyclic unsaturated C₃-C₁₄ moieties, exemplified but not limited to phenyl, biphenyl, naphthyl, pyridyl, furyl, thiophenyl, imidazoyl, pyrimidinyl, and oxazoyl; which may further be substituted with one to five members selected from the group consisting of hydroxy, C₁-C₈ alkoxy, C₁-C₈ branched or straight-chain alkyl, acyloxy, carbamoyl, amino, N-acylamino, nitro, halogen, trifluoromethyl, cyano, and carboxyl (see e.g. Katritzky, Handbook of Heterocyclic Chemistry). If not otherwise indicated, the term “aryl” includes unsubstituted, substituted, and/or heteroatom-containing aromatic substituents.

The term “aralkyl” refers to an alkyl group with an aryl substituent, and the term “alkaryl” refers to an aryl group with an alkyl substituent, wherein “alkyl” and “aryl” are as defined above. In general, aralkyl and alkaryl groups herein contain 6 to 30 carbon atoms. Aralkyl and alkaryl groups may, for example, contain 6 to 20 carbon atoms, and as a further example, such groups may contain 6 to 12 carbon atoms.

“Aryloxy” refers to the group —O-aryl, wherein aryl is as defined herein, including, by way of example, phenoxy, naphthoxy, and the like, including optionally substituted aryl groups as also defined herein.

“Amino” refers to the group —NH₂.

The term “substituted amino” refers to the group —NRR where each R is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl, and heterocyclyl provided that at least one R is not hydrogen.

The term “azido” or “azide” refers to the group —N₃.

“Carboxyl,” “carboxy” or “carboxylate” refers to —CO₂H or salts thereof.

“Carboxyl ester” or “carboxy ester” or the terms “carboxyalkyl” or “carboxylalkyl” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” or “carbonate” refers to the groups —O—C(O)O— alkyl, —O—C(O)O-substituted alkyl, —O—C(O)O-alkenyl, —O—C(O)O-substituted alkenyl, —O—C(O)O— alkynyl, —O—C(O)O-substituted alkynyl, —O—C(O)O-aryl, —O—C(O)O-substituted aryl, —O—C(O)O— cycloalkyl, —O—C(O)O-substituted cycloalkyl, —O—C(O)O-cycloalkenyl, —O—C(O)O-substituted cycloalkenyl, —O—C(O)O-heteroaryl, —O—C(O)O-substituted heteroaryl, —O—C(O)O-heterocyclic, and —O—C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” or “nitrile” refers to the group —CN.

As used herein, “carbocycle” or “carbocyclic ring” is intended to mean any stable monocyclic, bicyclic, or tricyclic ring having the specified number of carbons, any of which may be saturated, unsaturated, or aromatic. For example a C3-14 carbocycle is intended to mean a mono-, bi-, or tricyclic ring having 3, 4, 5, 6, 7, 8, 9, 10, 11, 12, 13, or 14 carbon atoms. Examples of carbocycles include, but are not limited to, cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, cyclooctadienyl, fluorenyl, phenyl, naphthyl, indanyl, adamantyl, and tetrahydronaphthyl. Bridged rings are also included in the definition of carbocycle, including, for example, [3.3.0]bicyclooctane, [4.3.0]bicyclononane, [4.4.0]bicyclodecane, and [2.2.2]bicyclooctane. A bridged ring occurs when a covalent bond or one or more carbon atoms link two non-adjacent carbon atoms in a ring. In one embodiment, bridge rings are one or two carbon atoms. It is noted that a bridge always converts a monocyclic ring into a bicyclic ring. When a ring is bridged, the substituents recited for the ring may also be present on the bridge. Fused (e.g., naphthyl and tetrahydronaphthyl) and spiro rings are also included.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclooctyl and the like. Such cycloalkyl groups include, by way of example, single ring structures such as cyclopropyl, cyclobutyl, cyclopentyl, cyclooctyl, and the like, or multiple ring structures such as adamantanyl, and the like.

The term “substituted cycloalkyl” refers to cycloalkyl groups having from 1 to 5 substituents, or from 1 to 3 substituents, selected from alkyl, substituted alkyl, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

“Cycloalkenyl” refers to non-aromatic cyclic alkyl groups of from 3 to 10 carbon atoms having single or multiple rings and having at least one double bond and preferably from 1 to 2 double bonds.

The term “substituted cycloalkenyl” refers to cycloalkenyl groups having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

“Cycloalkynyl” refers to non-aromatic cycloalkyl groups of from 5 to 10 carbon atoms having single or multiple rings and having at least one triple bond.

“Cycloalkoxy” refers to —O-cycloalkyl.

“Cycloalkenyloxy” refers to —O-cycloalkenyl.

“Halo” or “halogen” refers to fluoro, chloro, bromo, and iodo.

“Hydroxy” or “hydroxyl” refers to the group —OH.

“Heteroaryl” refers to an aromatic group of from 1 to 15 carbon atoms, such as from 1 to 10 carbon atoms and 1 to 10 heteroatoms selected from the group consisting of oxygen, nitrogen, and sulfur, selenium or tellurium within the ring. Such heteroaryl groups can have a single ring (such as, pyridinyl, imidazolyl or furyl) or multiple condensed rings in a ring system (for example as in groups such as, indolizinyl, quinolinyl, benzofuran, benzimidazolyl or benzothienyl), wherein at least one ring within the ring system is aromatic and at least one ring within the ring system is aromatic, provided that the point of attachment is through an atom of an aromatic ring. In certain embodiments, the nitrogen and/or sulfur ring atom(s) of the heteroaryl group are optionally oxidized to provide for the N-oxide (N→O), sulfinyl, or sulfonyl moieties. This term includes, by way of example, pyridinyl, pyrrolyl, indolyl, thiophenyl, and furanyl. Unless otherwise constrained by the definition for the heteroaryl substituent, such heteroaryl groups can be optionally substituted with 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl and —SO₂-heteroaryl, and trihalomethyl.

The term “heteroaralkyl” refers to the groups -alkylene-heteroaryl where alkylene and heteroaryl are defined herein. This term includes, by way of example, pyridylmethyl, pyridylethyl, indolylmethyl, and the like.

“Heteroaryloxy” refers to —O-heteroaryl.

“Heterocycle,” “heterocyclic,” “heterocycloalkyl,” and “heterocyclyl” refer to a saturated or unsaturated group having a single ring or multiple condensed rings, including fused bridged and spiro ring systems, and having from 3 to 20 ring atoms, including 1 to 10 hetero atoms. These ring atoms are selected from the group consisting of nitrogen, sulfur, or oxygen, selenium, tellurium for example, wherein, in fused ring systems, one or more of the rings can be cycloalkyl, aryl, or heteroaryl, provided that the point of attachment is through the non-aromatic ring. In certain embodiments, the nitrogen and/or sulfur atom(s) of the heterocyclic group are optionally oxidized to provide for the N-oxide, —S(O)—, or —SO₂— moieties.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, and the like.

Unless otherwise constrained by the definition for the heterocyclic substituent, such heterocyclic groups can be optionally substituted with 1 to 5, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO— alkyl, —SO-substituted alkyl, —SO-aryl, —SO-heteroaryl, —SO₂-alkyl, —SO₂-substituted alkyl, —SO₂-aryl, —SO₂-heteroaryl, and fused heterocycle.

By “sulfonate” is meant sulfonic acid, or salt of sulfonic acid. Similarly, by “carboxy” is meant carboxylic acid or salt of carboxylic acid. “Phosphate”, as used herein, is an ester of phosphoric acid, and includes salts of phosphate. “Phosphonate”, as used herein, means phosphonic acid and includes salts of phosphonate. As used herein, unless otherwise specified, the alkyl portions of substituents such as alkyl, alkoxy, arylalkyl, alylamino, dialkylamino, trialkylammonium, or perfluoroalkyl are optionally saturated, unsaturated, linear or branched, and all alkyl, alkoxy, alkylamino, and dialkylamino substituents are themselves optionally further substituted by carboxy, sulfonate, amino, or hydroxy.

“Nitro” refers to the group —NO₂.

“Oxo” refers to the atom (═O).

“Sulfonyl” refers to the group SO₂-alkyl, SO₂-substituted alkyl, SO₂-alkenyl, SO₂-substituted alkenyl, SO₂-cycloalkyl, SO₂-substituted cycloalkyl, SO₂-cycloalkenyl, SO₂-substituted cylcoalkenyl, SO₂-aryl, SO₂-substituted aryl, SO₂-heteroaryl, SO₂-substituted heteroaryl, SO₂-heterocyclic, and SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Sulfonyl includes, by way of example, methyl-SO₂—, phenyl-SO₂—, and 4-methylphenyl-SO₂—.

The term “sulfonate-containing group” refers to a group including SO₃— and RSO₃—, wherein R is substituted or unsubstituted alkyl, substituted or unsubstituted heteroalkyl, substituted or unsubstituted aryl, or substituted or unsubstituted heteroaryl.

“Thiol” refers to the group —SH.

“Thioxo” or the term “thioketo” refers to the atom (═S).

“Alkylthio” or the term “thioalkoxy” refers to the group —S-alkyl, wherein alkyl is as defined herein. In certain embodiments, sulfur may be oxidized to —S(O)—. The sulfoxide may exist as one or more stereoisomers.

The term “substituted thioalkoxy” refers to the group —S-substituted alkyl.

The term “thioaryloxy” refers to the group aryl-S— wherein the aryl group is as defined herein including optionally substituted aryl groups also defined herein.

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, —R⁶⁰, halo, ═O, —OR⁷⁰, —SR⁷⁰, —NR⁸⁰R⁸⁰, trihalomethyl, —CN, —OCN, —SCN, —NO, —NO₂, ═N₂, —N₃, —SO₂R⁷⁰, —SO₂O⁻M⁺, —SO₂OR⁷⁰, —OSO₂R⁷⁰, —OSO₂O⁻M⁺, —OSO₂OR⁷⁰, —P(O)(O⁻)₂(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —C(O)O⁻M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰ is selected from the group consisting of optionally substituted alkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R⁸⁰'s, taken together with the nitrogen atom to which they are bonded, form a 5-, 6- or 7-membered heterocycloalkyl which may optionally include from 1 to 4 of the same or different additional heteroatoms selected from the group consisting of O, N, Se, Te and S, of which N may have —H or C₁-C₃ alkyl substitution; and each M⁺ is a counter ion with a net single positive charge. Each M⁺ may independently be, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (“subscript 0.5 means that one of the counter ions for such divalent alkali earth ions can be an ionized form of a compound of the invention and the other a typical counter ion such as chloride, or two ionized compounds disclosed herein can serve as counter ions for such divalent alkali earth ions, or a doubly ionized compound of the invention can serve as the counter ion for such divalent alkali earth ions). As specific examples, —NR⁸⁰R⁸⁰ is meant to include —NH₂, —NH-alkyl, N-pyrrolidinyl, N-piperazinyl, 4N-methyl-piperazin-1-yl and N-morpholinyl.

In addition to the disclosure herein, substituent groups for hydrogens on unsaturated carbon atoms in “substituted” alkene, alkyne, aryl and heteroaryl groups are, unless otherwise specified, —R⁶⁰, halo, —O⁻M⁺, —OR⁷⁰, —SR⁷⁰, —S⁻M⁺, —NR⁸⁰R⁸⁰, trihalomethyl, —CF₃, —CN, —OCN, —SCN, —NO, —NO₂, —N₃, —SO₂R⁷⁰, —SO₃⁻M⁺, —SO₃R⁷⁰, —OSO₂R⁷⁰, —OSO₃⁻M⁺, —OSO₃R⁷⁰, —PO₃⁻²(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO₂R⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰, or —S⁻M⁺.

In addition to the groups disclosed with respect to the individual terms herein, substituent groups for hydrogens on nitrogen atoms in “substituted” heteroalkyl and cycloheteroalkyl groups are, unless otherwise specified, —R⁶⁰, —O⁻M⁺, —OR⁷⁰, —SR⁷⁰, —S⁻M⁺, —NR⁸⁰R⁸⁰, trihalomethyl, —CF₃, —CN, —NO, —NO₂, —S(O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O) NR⁸⁰R⁸⁰, —NR⁷⁰C(NR⁷⁰)R⁷⁰ and —NR⁷⁰C(NR⁷)NR⁸⁰R⁸⁰, where R⁶⁰, R⁷⁰, R⁸⁰ and M⁺ are as previously defined.

In addition to the disclosure herein, in a certain embodiment, a group that is substituted has 1, 2, 3, or 4 substituents, 1, 2, or 3 substituents, 1 or 2 substituents, or 1 substituent.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups specifically contemplated herein are limited to substituted aryl-(substituted aryl)-substituted aryl.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

The term “pharmaceutically acceptable salt” means a salt which is acceptable for administration to a patient, such as a mammal (salts with counterions having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, formate, tartrate, besylate, mesylate, acetate, maleate, oxalate, and the like.

“Pharmaceutically effective amount” and “therapeutically effective amount” refer to an amount of a compound sufficient to elicit the desired therapeutic effect (e.g., treatment of a specified disorder or disease or one or more of its symptoms and/or prevention of the occurrence of the disease or disorder). In reference to polyglutamine diseases, a pharmaceutically or therapeutically effective amount includes an amount sufficient to, among other things, prevent or cause a reduction of proteinaceous deposits in the brain of a subject.

The term “salt thereof” means a compound formed when a proton of an acid is replaced by a cation, such as a metal cation or an organic cation and the like. Where applicable, the salt is a pharmaceutically acceptable salt, although this is not required for salts of intermediate compounds that are not intended for administration to a patient. By way of example, salts of the present compounds include those wherein the compound is protonated by an inorganic or organic acid to form a cation, with the conjugate base of the inorganic or organic acid as the anionic component of the salt.

“Solvate” refers to a complex formed by combination of solvent molecules with molecules or ions of the solute. The solvent can be an organic compound, an inorganic compound, or a mixture of both. Some examples of solvents include, but are not limited to, methanol, N,N-dimethylformamide, tetrahydrofuran, dimethylsulfoxide, and water. When the solvent is water, the solvate formed is a hydrate.

“Stereoisomer” and “stereoisomers” refer to compounds that have same atomic connectivity but different atomic arrangement in space. Stereoisomers include cis-trans isomers, E and Z isomers, enantiomers, and diastereomers.

“Tautomer” refers to alternate forms of a molecule that differ only in electronic bonding of atoms and/or in the position of a proton, such as enol-keto and imine-enamine tautomers, or the tautomeric forms of heteroaryl groups containing a —N═C(H)—NH— ring atom arrangement, such as pyrazoles, imidazoles, benzimidazoles, triazoles, and tetrazoles. A person of ordinary skill in the art would recognize that other tautomeric ring atom arrangements are possible.

By the term “functional groups” is meant chemical groups such as halo, hydroxyl, sulfhydryl, C1-C24 alkoxy, C2-C24 alkenyloxy, C2-C24 alkynyloxy, C5-C20 aryloxy, acyl (including C2-C24 alkylcarbonyl (—CO-alkyl) and C6-C20 arylcarbonyl (—CO-aryl)), acyloxy (—O-acyl), C2-C24 alkoxycarbonyl (—(CO)—O-alkyl), C6-C20 aryloxycarbonyl (—(CO)—O-aryl), halocarbonyl (—CO)—X where X is halo), C2-C24 alkylcarbonato (—O—(CO)—O-alkyl), C6-C20 arylcarbonato (—O—(CO)—O-aryl), carboxy (—COOH), carboxylato (—COO—), carbamoyl (—(CO)—NH₂), mono-substituted C1-C24 alkylcarbamoyl (—(CO)—NH(C1-C24 alkyl)), di-substituted alkylcarbamoyl (—(CO)—N(C1-C24 alkyl)₂), mono-substituted arylcarbamoyl (—(CO)—NH-aryl), thiocarbamoyl (—(CS)—NH₂), carbamido (—NH—(CO)—NH₂), cyano (—C≡N), isocyano (—N+≡C—), cyanato (—O—C≡N), isocyanato (—O—N+≡C—), isothiocyanato (—S—C≡N), azido (—N═N+=N—), formyl (—(CO)—H), thioformyl (—(CS)—H), amino (—NH₂), mono- and di-(C1-C24 alkyl)-substituted amino, mono- and di-(C5-C20 aryl)-substituted amino, C2-C24 alkylamido (—NH—(CO)-alkyl), C5-C20 arylamido (—NH—(CO)-aryl), imino (—CR═NH where R=hydrogen, C1-C24 alkyl, C5-C20 aryl, C6-C20 alkaryl, C6-C20 aralkyl, etc.), alkylimino (—CR═N(alkyl), where R=hydrogen, alkyl, aryl, alkaryl, etc.), arylimino (—CR═N(aryl), where R=hydrogen, alkyl, aryl, alkaryl, etc.), nitro (—NO₂), nitroso (—NO), sulfo (—SO₂—OH), sulfonato (—SO₂-0-), C1-C24 alkylsulfanyl (—S-alkyl; also termed “alkylthio”), arylsulfanyl (—S-aryl; also termed “arylthio”), C1-C24 alkylsulfinyl (—(SO)-alkyl), C5-C20 arylsulfinyl (—(SO)-aryl), C1-C24 alkylsulfonyl (—SO2-alkyl), C5-C20 arylsulfonyl (—SO₂-aryl), phosphono (—P(O)(OH)₂), phosphonato (—P(O)(O—)₂), phosphinato (—P(O)(O—)), phospho (—PO₂), and phosphino (—PH₂), mono- and di-(C1-C24 alkyl)-substituted phosphino, mono- and di-(C5-C20 aryl)-substituted phosphine. In addition, the aforementioned functional groups may, if a particular group permits, be further substituted with one or more additional functional groups or with one or more hydrocarbyl moieties such as those specifically enumerated above.

Nitrogen-containing heterocycles of interest that find use as hydrophilic moieties include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, and substituted versions thereof.

As used herein the term “PEG” refers to a polyethylene glycol or a modified polyethylene glycol. Modified polyethylene glycol polymers include a methoxypolyethylene glycol, and polymers that are unsubstituted or substituted at one end with an alkyl, a substituted alkyl or a functional group (e.g., as described herein). Any convenient linking groups may be utilized at the terminal of a PEG to connect the group to a moiety of interest.

The term “cyanine” means a family of cyanine dyes, Cy2, Cy3, Cy5, Cy7, and their derivatives, based on the partially saturated indole nitrogen heterocyclic nucleus with two aromatic units being connected via a polyalkene bridge of varying carbon number. These probes exhibit fluorescence excitation and emission profiles that are similar to many of the traditional dyes, such as fluorescein and tetramethylrhodamine, but with enhanced water solubility, photostability, and higher quantum yields. Most of the cyanine dyes are more environmentally stable than their traditional counterparts, rendering their fluorescence emission intensity less sensitive to pH and organic mounting media. The excitation wavelengths of the Cy series of synthetic dyes are tuned specifically for use with common laser and arc-discharge sources, and the fluorescence emission can be detected with traditional filter combinations. Marketed by a number of distributors, the cyanine dyes are readily available as reactive dyes or fluorophores coupled to a wide variety of secondary antibodies, dextrin, streptavidin, and egg-white avidin.

The term “Near-infrared” or “NIR” refers to wavelengths within the range of 650-2500 nm. Unless otherwise specified, these terms as used herein refer to wavelengths within the range of 650-900 nm.

It will be appreciated that the term “or a salt or solvate or stereoisomer thereof” is intended to include all permutations of salts, solvates and stereoisomers, such as a solvate of a pharmaceutically acceptable salt of a stereoisomer of subject compound.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

In certain embodiments, a substituent may contribute to optical isomerism and/or stereo isomerism of a compound. Salts, solvates, hydrates, and prodrug forms of a compound are also of interest. All such forms are embraced by the present disclosure. Thus the compounds described herein include salts, solvates, hydrates, prodrug and isomer forms thereof, including the pharmaceutically acceptable salts, solvates, hydrates, prodrugs and isomers thereof. In certain embodiments, a compound may be a metabolized into a pharmaceutically active derivative.

Unless otherwise specified, reference to an atom is meant to include isotopes of that atom. For example, reference to His meant to include ¹H, ²H (i.e., D) and ³H (i.e., T), and reference to C is meant to include ¹²C and all isotopes of carbon (such as ¹³C).

The term “sample” as used herein relates to a material or mixture of materials, typically, although not necessarily, in fluid, i.e., aqueous, form, containing one or more components of interest. Samples may be derived from a variety of sources such as from food stuffs, environmental materials, a biological sample or solid, such as tissue or fluid isolated from an individual, including but not limited to, for example, plasma, serum, spinal fluid, semen, lymph fluid, the external sections of the skin, respiratory, intestinal, and genitourinary tracts, tears, saliva, milk, blood cells, tumors, organs, and also samples of in vitro cell culture constituents (including but not limited to conditioned medium resulting from the growth of cells in cell culture medium, putatively virally infected cells, recombinant cells, and cell components). In certain embodiments of the method, the sample includes a cell. In some instances of the method, the cell is in vitro. In some instances of the method, the cell is in vivo.

The terms “individual,” “subject,” “host,” and “patient,” used interchangeably herein, refer to an individual organism, e.g., a mammal, including, but not limited to, murines, simians, non-human primates, humans, mammalian farm animals, mammalian sport animals, and mammalian pets.

The term “treating” or “treatment” as used herein means the treating or treatment of a disease or medical condition in a patient, such as a mammal (particularly a human) that includes: (a) preventing the disease or medical condition from occurring, such as, prophylactic treatment of a subject; (b) ameliorating the disease or medical condition, such as, eliminating or causing regression of the disease or medical condition in a patient; (c) suppressing the disease or medical condition, for example by, slowing or arresting the development of the disease or medical condition in a patient; or (d) alleviating a symptom of the disease or medical condition in a patient.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a functional dye” includes a plurality of such functional dyes known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Definitions of other terms and concepts appear throughout the detailed description below.

### Compounds and Methods of Use

The present disclosure provides functional dyes, which are useful for highlighting premalignant lesions and malignant lesions in an individual. The present disclosure provides compositions comprising a functional dye. The present disclosure provides methods of administering a functional dye to an individual by oral or topical administration to high-light premalignant or malignant lesions in the individual. The functional dyes, compositions and methods find use in a variety of applications in which highlighting of benign or premalignant lesions (e.g. an adenoma) or malignant lesions is desired.

### Functional Dyes

The present disclosure provides functional dyes, which find use in a variety of applications. For example, the subject dyes may find use in specifically highlighting incipient colorectal cancers (CRC) during endoscopic surveillance following oral administration. In some cases, the functional dyes include a clinically-applied near-infrared fluorescent (NIRF) dye-backbone, conjugated to a small molecule that targets a protein. In some cases, the NIRF dye-backbone is conjugated to an premalignant lesion-targeting moiety. In certain cases, the NIRF is a cyanine dye-backbone. In certain cases, the targeting moiety includes a hetero containing functional group. In certain cases, the targeting moiety includes a morpholine functional group. Exemplary functional dyes including a cyanine dye-backbone are set forth in the following structures 1-8 and formulae A1-A2 or I-III.

In some cases, the subject functional dye is of the formula:

D-L-F

wherein:
D is a near infrared fluorescent dye;
L is an optional linker; and
F is a small molecule that targets a protein.

In some embodiments of the subject functional dye, the near infrared fluorescent dye is a cyanine (Cy) dye. In certain cases, the dye is a commercially available dye, including but not limited to, IR780 (CAS number 207399-07-3), IR783 (CAS number 115970-66-6), S0456 (CAS number 1252007-83-2), S2180 (CAS number 162093-44-9) and S2493 (2-[2-(2-chloro-3-[2-[1,1-dimethyl-7-sulfo-3-(4-sulfobutyl)-1,3-dihydro-benzo[e]indol-2-ylidene]-ethylidene]-cyclopent-1-enyl)-vinyl]-1,1-dimethyl-7-sulfo-3-(4-sulfobutyl)-1H-benzo[e]indolium hydroxide, inner salt, triethylammonium salt, available from FEW Chemicals GmbH). In certain cases, the dye is a derivative of a commercially available dye. In certain cases, the dye is a water-soluble cyanine dye.

In some embodiments, the near infrared fluorescent dye is a cyanine dye of the formula (A):

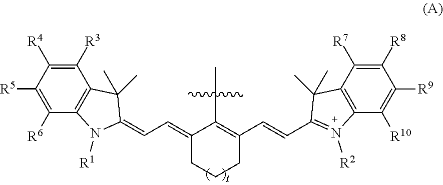

wherein:
R¹ and R² are each independently selected from H, alkyl, and (CH₂)ₙR¹², wherein n is an integer from 1 to 20 and R¹² is selected from the group consisting of H, sulfonate, carboxylic acid, amine, quaternary ammonium cation, phosphate, ester, halogen, azide, cyano, alkyne, and heterocycle; R³ and R⁴, are each independently selected from H, alkyl, substituted alkyl, alkoxy, substituted alkoxy, halogen, CF₃, carboxy, phosphonate, carboxylate, cyano, hydroxyl, carboxyamide, sulfonamide, sulfonate, amino, substituted amino, amide, aryl, substituted aryl, heteroaryl, substituted heteroaryl, heterocycle and substituted heterocycle;
or R³ and R⁴ together with the carbon to which they are attached form a group selected from aryl, substituted aryl, heteroaryl, substituted heteroaryl, heterocycle, substituted heterocycle, cycloalkyl and substituted cycloalkyl;
R⁷ and R⁸ are each independently selected from H, alkyl, substituted alkyl, alkoxy, substituted alkoxy, halogen, CF₃, carboxy, phosphonate, carboxylate, cyano, hydroxyl, carboxyamide, sulfonamide, sulfonate, amino, substituted amino, amide, aryl, substituted aryl, heteroaryl, substituted heteroaryl, heterocycle and substituted heterocycle;
or R⁷ and R⁸ together with the carbon to which they are attached form a group selected from aryl, substituted aryl, heteroaryl, substituted heteroaryl, heterocycle, substituted heterocycle, cycloalkyl and substituted cycloalkyl;
R⁵, R⁶, R⁹ and R¹⁰ are each independently selected from H, alkyl, substituted alkyl, alkoxy, substituted alkoxy, halogen, CF₃, carboxy, phosphonate, carboxylate, cyano, hydroxyl, carboxyamide, sulfonamide, sulfonate, amino, substituted amino, amide, aryl, substituted aryl, heteroaryl, substituted heteroaryl, heterocycle and substituted heterocycle; and
t is an integer from 0 to 1.

In certain embodiments of the dye of formula (A), the dye is substituted with at least one iodide. In certain cases of the dye of formula (A), t is 0, such that the central cyclic group is a 5-membered ring, e.g., a pentene ring. In certain other cases of the dye of formula (A), t is 1, such that the central cyclic group is a 6-membered ring, e.g., a cyclohexene.

In certain embodiments, the dye of formula (A) is a water-soluble dye, e.g., the dye of formula (A) contains one or more water-soluble groups. In certain embodiments of the dye of formula (A), any one or more of R¹ to R¹⁰ comprises a water-soluble group. In certain embodiments, at least two of R¹ to R¹⁰ comprise a water-soluble group. In certain embodiments, at least three of R¹ to R¹⁰ comprise a water-soluble group. In certain embodiments, at least four of R¹ to R¹⁰ comprise a water-soluble group. The term “water-soluble group” (WSG) refers to a group that is well solvated in aqueous environments and that imparts improved water solubility upon the dye/molecules to which it is attached. In some instances, the dyes incorporate at least one charged group to increase water solubility. Any convenient charged groups may be incorporated. Charged groups of interest, include but are not limited to, a sulfonate, an ammonium, a carboxy, a phosphate, an amino, a substituted amino and the like. The term “sulfonate”, by itself or as part of another group, refers to any compound or substituent that contains sulfonic acid, a salt thereof, e.g., one or more moieties having the following structure:

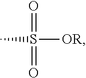

where R is hydrogen or a counter ion, such as a metal ion or ammonium ion. Similarly, by “carboxy” is meant carboxylic acid or salt of carboxylic acid. “Phosphate”, as used herein, is an ester of phosphoric acid, and includes salts of phosphate. “Phosphonate”, as used herein, means phosphonic acid and includes salts of phosphonate. WSG of interest include, but are not limited to, carboxylate, phosphonate, phosphate, sulfonate, sulfate, sulfinate, sulfonium, ester, polyethylene glycols (PEG) and modified PEGs, hydroxyl, amine, ammonium, guanidinium, pyridinium, polyamine and sulfonium, polyalcohols, straight chain or cyclic saccharides, primary, secondary, tertiary, or quaternary amines and polyamines, phosphonate groups, phosphinate groups, ascorbate groups or glycols.

While the increase in solubility may vary, in some instances the increase (as compared to the dye without the WSG(s)) is 2-fold or more, e.g., 5-fold, 10-fold, 25-fold, 50-fold, 100-fold or more. In some embodiments, a WSG increases the solubility of the dye compound in a predominantly aqueous solution, as compared to a control dye which lacks the WSG. In some instances, the WSGs of the dye are non-ionic side groups capable of imparting solubility in water in excess of 10 mg/mL. The water solubilizing groups may be any convenient hydrophilic group that is well solvated in aqueous environments. In some cases, the hydrophilic water solubilizing group is charged, e.g., positively or negatively charged. In certain cases, the hydrophilic water solubilizing group is a neutral hydrophilic group. In some embodiments, the WSG is a hydrophilic polymer, e.g., a polyethylene glycol, a cellulose, a chitosan, or a derivative thereof. Multiple WSGs may be included in the subject dyes. In some embodiments, at least one WSG on the subject dye is a sulfonate group. In some embodiments, at least two WSGs on the subject dye are sulfonate groups. In some embodiments, at least three WSGs on the subject dye are sulfonate groups. In some embodiments, at least four WSGs on the subject dye are sulfonate groups.

In certain embodiments the dye of formula (A) is selected from the following structures:

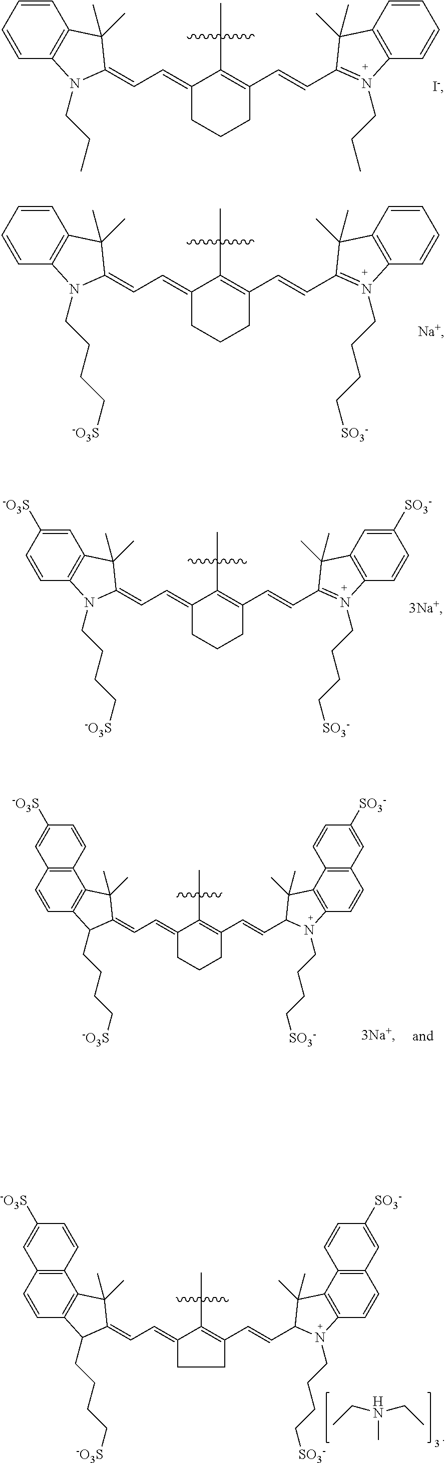

In certain embodiments, the near infrared fluorescent dye is fluorescein or a derivative thereof. In some cases, the fluorescein derivative is fluorescein isothiocyanate (FITC).

In certain embodiments, the near infrared fluorescent dye is imaged in the first near-infrared window (NIR-I), from 700 nm to 1000 nm, such as 700 nm, 725 nm, 750 nm, 775 nm, 800 nm, 825 nm, 850 nm, 875 nm, 900 nm, 925 nm, 950 nm, 975 nm or 1000 nm. In some cases, the dye is imaged at 800 nm or more, such as 825 nm or more, 850 nm or more, 900 nm or more, 950 nm or more, or even more. In certain embodiments, the near infrared fluorescent dye is imaged in the second near-infrared window (NIR-II), from 1000 nm to 1700 nm, such as 1000 nm, 1100 nm, 1200 nm, 1300 nm, 1400 nm, 1500 nm, 1600 nm or 1700 nm. In certain cases, the dye is imaged at 1000 nm or more, such as 1100 nm or more, 1200 nm or more, 1300 nm or more, 1400 nm or more, 1500 nm or more, 1600 nm or more, 1700 nm or more, or even more.

In certain embodiments, the subject functional dye comprises a linker (L) between the near infrared fluorescent dye (D) and the small molecule that targets a protein (F). By “linker” as in “linking group,” “linker moiety,” etc., is meant a linking moiety that connects two groups via covalent bonds. The linker may be linear, branched, cyclic or a single atom. Examples of such linking groups include alkyl, alkenylene, alkynylene, arylene, alkarylene, aralkylene, and linking moieties containing functional groups including, without limitation: amido (—NH—CO—), ureylene (—NH—CO—NH—), imide (—CO—NH—CO—), epoxy (—O—), epithio (—S—), epidioxy (—O—O—), carbonyldioxy (—O—CO—O—), alkyldioxy (—O—(CH2)n-O—), epoxyimino (—O—NH—), epimino (—NH—), carbonyl (—CO—), etc. In certain cases, one, two, three, four or five or more carbon atoms of a linker backbone may be optionally substituted with a sulfur, nitrogen or oxygen heteroatom. The bonds between backbone atoms may be saturated or unsaturated, usually not more than one, two, or three unsaturated bonds will be present in a linker backbone. The linker may include one or more substituent groups, for example with an alkyl, aryl or alkenyl group. A linker may include, without limitations, poly(ethylene glycol) unit(s) (e.g., —(CH₂—CH₂—O)—); ethers, thioethers, amines, alkyls (e.g., (C₁-C₁₂)alkyl), which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle or a cycloalkyl group, where 2 or more atoms, e.g., 2, 3 or 4 atoms, of the cyclic group are included in the backbone. A linker may be cleavable or non-cleavable. Any convenient orientation and/or connections of the linkers to the linked groups may be used.

In certain cases, the linker (L) comprises a group selected from alkyl, substituted alkyl, alkenyl, substituted alkenyl, alkynyl, substituted alkynyl, acyl, substituted acyl, C₁-C₁₂ alkoxy, substituted C₁-C₁₂ alkoxy, cycloalkyl, substituted cycloalkyl, heterocycloalkyl, substituted heterocycloalkyl, aryl, substituted aryl, heteroaryl and substituted heteroaryl. In some cases, L comprises an alkyl or a substituted alkyl group. In some cases, L comprises an alkenyl or a substituted alkenyl group. In some cases, L comprises an alkynyl or a substituted alkynyl group. In some cases, L comprises an acyl or a substituted acyl group. In some cases, L comprises a C₁-C₁₂ alkoxy or a substituted C₁-C₁₂ alkoxy group. In some cases, L comprises a cycloalkyl or a substituted cycloalkyl. In some cases, L comprises a heterocycloalkyl or a substituted heterocycloalkyl. In some cases, L comprises an aryl or a substituted aryl. In some cases, L comprises a heteroaryl or a substituted heteroaryl. In some cases, the linker includes an amino, or a thiol group. In some cases, the linker includes a polyalkene group. In some cases, the linker includes a piperazine group. In some cases, the linker includes an aniline group.

In certain cases of the subject functional dye, the linker (L) is absent such that the infrared fluorescent dye (D) is directly bonded to the small molecule that targets a protein (F). In certain embodiments, the target protein is selected from a wild type, a splice variant, a dominant negative and a mutant protein. In certain cases, (F) targets extracellular proteins. In other cases, (F) targets cell-associated proteins. In certain cases, (F) targets extracellular domains (ECD) of cell surface proteins. In certain other cases, (F) targets intracellular domains of (trans)membrane proteins. In certain cases, (F) targets intracellular proteins. In certain cases, the target protein is expressed. In certain cases, the target protein is overexpressed. In certain cases, the target protein is hyperactive. In certain cases, the target protein is silenced.

In certain cases of the subject functional dye, the small molecule that targets a protein (F) includes the following fragment

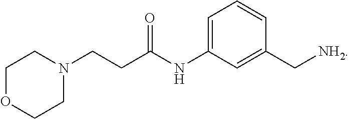 [SMILES: NCc1cccc(NC(=O)CCN2CCOCC2)c1]

In certain cases of the subject functional dye, the small molecule that targets a protein (F) is a fragment of a known drug that targets a protein selected from the group consisting of (non)-receptor kinases, immune checkpoint proteins, G-protein-coupled receptors (GPCRs), influx transporters, efflux transporters, ion channels, human leukocyte antigens (HLA), proteases, caspases and nuclear receptors. In some cases, the fragment is an impurity of a known drug, such as a known impurity obtained during the synthetic process or during the degradation of the drug. In some cases, the impurity is a known compound for which a reference standard is commercially available. In some cases, the fragment is a metabolite of the drug. In certain cases, the metabolite a known compound and in some cases is commercially available.

In certain embodiments of the subject functional dye, the small molecule that targets a protein (F) includes an ion channel agonist or antagonist or fragment thereof. In certain cases, the ion channel antagonist is procainamide.

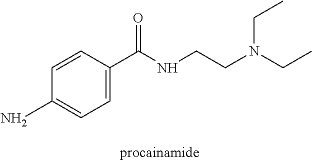 [SMILES: CCN(CC)CCNC(=O)c1ccc(N)cc1]

In certain embodiments of the subject functional dye, the small molecule that targets a protein (F) includes a small molecule kinase inhibitor, a small molecule kinase inhibitor impurity, a small molecule kinase inhibitor fragment or a small molecule kinase inhibitor metabolite. In certain cases, the small molecule kinase inhibitor is selected from imatinib, bosutinib, nintedanib, ponatinib, brigatinib and dasatinib. In certain cases, the small molecule kinase inhibitor is selected from ceritinib and palbociclib.

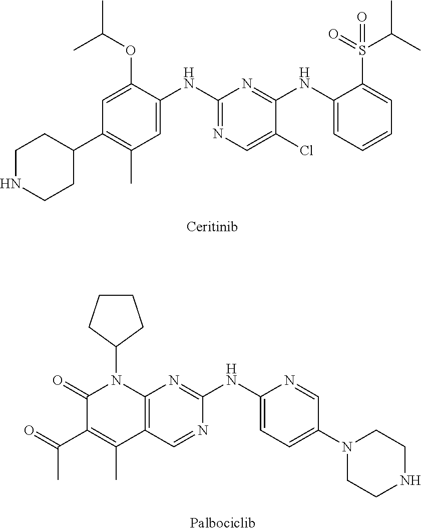 [SMILES: Cc1cc(Nc2ncc(Cl)c(Nc3ccccc3S(=O)(=O)C(C)C)n2)c(OC(C)C)cc1C1CCNCC1 ; CC(=O)c1c(C)c2cnc(Nc3ccc(N4CCNCC4)cn3)nc2n(C2CCCC2)c1=O]

In certain embodiments of the subject functional dye, the small molecule that targets a protein (F) includes a small molecule kinase metabolite selected from N-desmethyl imatinib, N-desmethyl bosutinib, N-desmethyl nintedanib, N-desmethyl ponatinib, N-desmethyl brigatinib, N-deshydroxyethyl dasatinib.

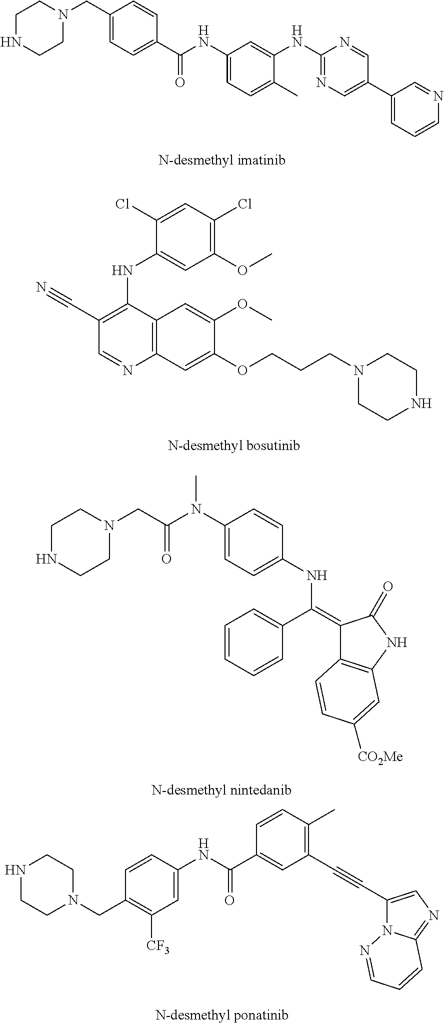 [SMILES: Cc1ccc(NC(=O)c2ccc(CN3CCNCC3)cc2)cc1Nc1ncc(-c2cccnc2)cn1 ; COc1cc(Nc2c(C#N)cnc3cc(OCCCN4CCNCC4)c(OC)cc23)c(Cl)cc1Cl ; COC(=O)c1ccc2c(c1)NC(=O)/C2=C(\Nc1ccc(N(C)C(=O)CN2CCNCC2)cc1)c1ccccc1 ; Cc1ccc(C(=O)Nc2ccc(CN3CCNCC3)c(C(F)(F)F)c2)cc1C#Cc1cnc2cccnn12]

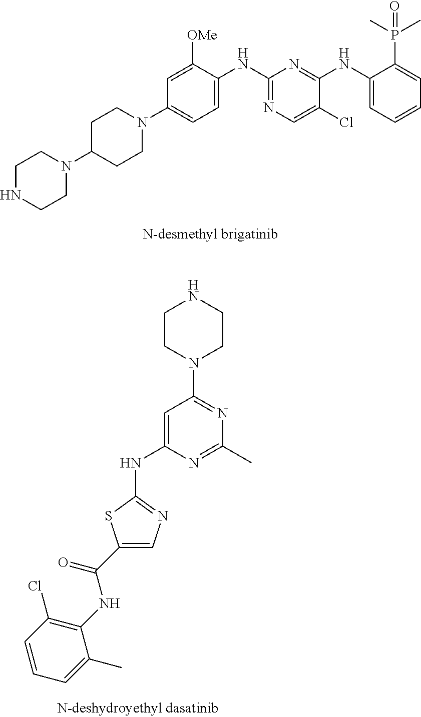 [SMILES: COc1cc(N2CCC(N3CCNCC3)CC2)ccc1Nc1ncc(Cl)c(Nc2ccccc2P(C)(C)=O)n1 ; Cc1nc(Nc2ncc(C(=O)Nc3c(C)cccc3Cl)s2)cc(N2CCNCC2)n1]

In certain embodiments of the subject functional dye, the small molecule that targets a protein (F) includes the small molecule kinase inhibitor erlotinib, or its metabolite N-desmethyl-erlotinib.

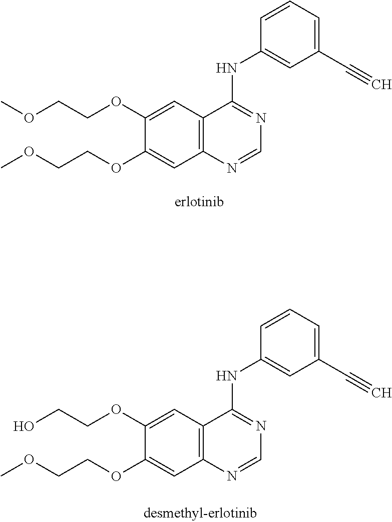

In certain embodiments of the subject functional dye, the small molecule that targets a protein (F) includes a fragment or impurity of nintedanib or lapatinib. In some cases, the fragment or impurity of nintedanib or lapatinib is selected from:

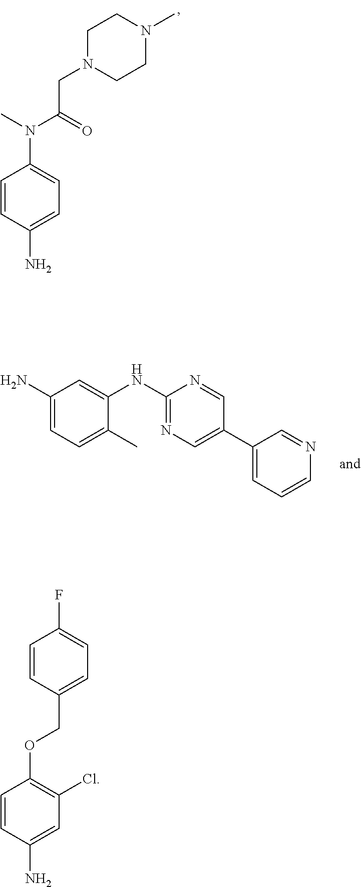 [SMILES: CN1CCN(CC(=O)N(C)c2ccc(N)cc2)CC1 ; Cc1ccc(N)cc1Nc1ncc(-c2cccnc2)cn1 ; Nc1ccc(OCc2ccc(F)cc2)c(Cl)c1]

In some embodiments of the subject functional dye, the small molecule that targets a protein (F) includes a fragment of a small molecule modulator of G-protein-coupled receptors (GPCRs). In some cases, the small molecule modulator of GPCRs is selected from sildenafil and eszopiclone. In some cases, the fragment is an impurity of a small molecule modulator of GPCRs. In some cases, the fragment is a metabolite of a small molecule modulator of GPCRs. In some cases, the metabolite of the small molecule modulator of G-protein-coupled receptors (GPCRs) is selected from N-desmethyl sildenafil and N-desmethyl eszopiclone.

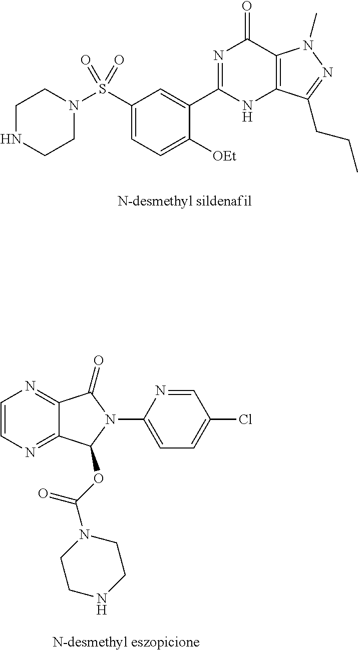 [SMILES: CCCc1nn(C)c2c(=O)nc(-c3cc(S(=O)(=O)N4CCNCC4)ccc3OCC)[nH]c12 ; O=C(O[C@H]1c2nccnc2C(=O)N1c1ccc(Cl)cn1)N1CCNCC1]

In some embodiments of the subject functional dye, the small molecule that targets a protein (F) includes an androgen receptor inhibitor or a fragment of an androgen receptor inhibitor. In some cases, the androgen receptor inhibitor is enzalutamide. In some cases, the fragment of the androgen receptor inhibitor is N-desmethyl-enzalutamide.

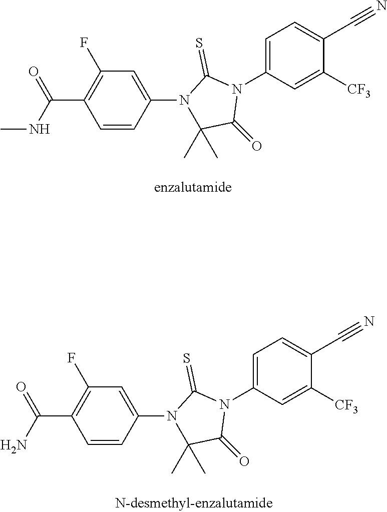 [SMILES: CNC(=O)c1ccc(N2C(=S)N(c3ccc(C#N)c(C(F)(F)F)c3)C(=O)C2(C)C)cc1F ; CC1(C)C(=O)N(c2ccc(C#N)c(C(F)(F)F)c2)C(=S)N1c1ccc(C(N)=O)c(F)c1]

In some embodiments of the subject functional dye, the small molecule that targets a protein (F) includes immunecheckpoint inhibitor such as programmed cell death ligand 1 inhibitor (PD-L1 inhibitor), or a fragment of a PD-L1 inhibitor. In some cases, the PD-L1 inhibitor is a compound of structure (9), or a fragment thereof:

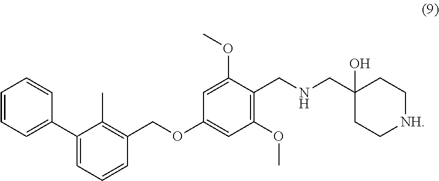 [SMILES: COc1cc(OCc2cccc(-c3ccccc3)c2C)cc(OC)c1CNCC1(O)CCNCC1]

In some embodiments of the subject functional dye, the small molecule that targets a protein (F) is of the formula (F1):

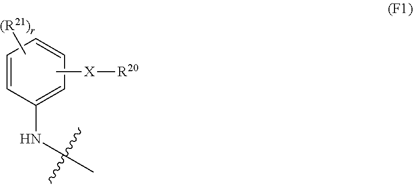

wherein:
X is C(O)NR², NR²², O or S, wherein R²² is selected from H, alkyl, substituted alkyl, aryl, substituted aryl, heterocycle, substituted heterocycle, heteroaryl and substituted heteroaryl;
R²⁰ is selected from substituted alkyl, substituted acyl, benzyl, substituted benzyl, aryl, substituted aryl, heteroaryl, substituted heteroaryl, heterocycle, substituted heterocycle;
R²¹ are each independently selected from alkyl, substituted alkyl, alkoxy, substituted alkoxy, halogen, CF₃, sulfonate, amino, substituted amino, amide, aryl, substituted aryl, heteroaryl, substituted heteroaryl, heterocycle and substituted heterocycle; and r is an integer from 0 to 4.

In certain embodiments of formula (F1), X is NR²². In certain cases, X is NR²² and R²² is H. In certain cases, X is NR²² and R²² is alkyl or substituted alkyl. In certain cases, the alkyl group is a methyl group. In certain cases, X is NR²² and R²² is aryl or substituted aryl. In certain cases, X is NR²² and R²² is a heterocycle or substituted heterocycle. In certain cases, X is NR²² and R²² is heteroaryl or substituted heteroaryl. In certain embodiments of formula (F1), X is C(O)NR²². In certain cases, X is C(O)NR²² and R²² is H. In certain cases, X is C(O)NR²² and R²² is alkyl or substituted alkyl. In certain cases, the alkyl group is a methyl group. In certain cases, X is C(O)NR²² and R²² is aryl or substituted aryl. In certain cases, X is C(O)NR²² and R²² is a heterocycle or substituted heterocycle. In certain cases, X is C(O)NR²² and R²² is heteroaryl or substituted heteroaryl. In certain embodiments of formula (F1), X is O. In certain other embodiments of formula (F1), X is S.

In certain embodiments of formula (F1), R²⁰ is substituted alkyl. In certain cases, R²⁰ is substituted acyl. In certain cases, R²⁰ is substituted acyl. In certain cases, R²⁰ is benzyl or substituted benzyl. In certain cases, R²⁰ is aryl or substituted aryl. In certain cases, R²⁰ is heteroaryl or substituted heteroaryl. In certain cases, R²⁰ is heterocycle or substituted heterocycle.

In certain embodiments of formula (F1), X is NR²², R²² is H or alkyl, and R²⁰ is substituted alkyl. In certain embodiments of formula (F1), X is NR²², R²² is H or alkyl, and R²⁰ is substituted acyl. In certain embodiments of formula (F1), X is NR²², R²² is H or alkyl, and R²⁰ is substituted benzyl or substituted benzyl. In certain embodiments of formula (F1), X is NR²², R²² is H or alkyl, and R²⁰ is aryl substituted aryl. In certain embodiments of formula (F1), X is NR²², R²² is H or alkyl, and R²⁰ is heteroaryl or substituted heteroaryl. In certain embodiments of formula (F1), X is NR²², R²² is H or alkyl, and R²⁰ is heterocycle or substituted heterocycle.

In certain embodiments of formula (F1), X is C(O)NR²², R²² is H or alkyl, and R²⁰ is substituted alkyl. In certain embodiments of formula (F1), X is C(O)NR²², R²² is H or alkyl, and R²⁰ is substituted acyl. In certain embodiments of formula (F1), X is C(O)NR²², R²² is H or alkyl, and R²⁰ is substituted benzyl or substituted benzyl. In certain embodiments of formula (F1), X is NR²², C(O)R²² is H or alkyl, and R²⁰ is aryl substituted aryl. In certain embodiments of formula (F1), X is C(O)NR²², R²² is H or alkyl, and R²⁰ is heteroaryl or substituted heteroaryl. In certain embodiments of formula (F1), X is C(O)NR²², R²² is H or alkyl, and R²⁰ is heterocycle or substituted heterocycle.

In certain embodiments of formula (F1), X is O, and R²⁰ is substituted alkyl. In certain embodiments of formula (F1), X is O, and R²⁰ is substituted acyl. In certain embodiments of formula (F1), X is O, and R²⁰ is substituted benzyl or substituted benzyl. In certain embodiments of formula (F1), X is O, and R²⁰ is aryl substituted aryl. In certain embodiments of formula (F1), X is O, and R²⁰ is heteroaryl or substituted heteroaryl. In certain embodiments of formula (F1), X is O, and R²⁰ is heterocycle or substituted heterocycle.

In certain embodiments of formula (F1), X is S, and R²⁰ is substituted alkyl. In certain embodiments of formula (F1), X is S, and R²⁰ is substituted acyl. In certain embodiments of formula (F1), X is S, and R²⁰ is substituted benzyl or substituted benzyl. In certain embodiments of formula (F1), X is S, and R²⁰ is aryl substituted aryl. In certain embodiments of formula (F1), X is S, and R²⁰ is heteroaryl or substituted heteroaryl. In certain embodiments of formula (F1), X is S, and R²⁰ is heterocycle or substituted heterocycle.

In certain embodiments of formula (F1), r is 0 such that the compound of formula (F1) contains no R²¹ substituents. In other cases, r is 1 such that the compound of formula (F1) contains one R²¹ substituent. In other cases, r is 2 such that the compound of formula (F1) contains two R²¹ substituents. In other cases, r is 3 such that the compound of formula (F1) contains three R²¹ substituents. In other cases, r is 4 such that the compound of formula (F1) contains four R²¹ substituents.

In certain embodiments of formula (F1), at least one R²¹ substituent is alkyl or substituted alkyl. In certain embodiments, at least one R²¹ substituent is alkoxy or substituted alkoxy. In certain embodiments, at least one R²¹ substituent is halogen. In certain embodiments, at least one R²¹ substituent is CF₃. In certain embodiments, at least one R²¹ substituent is sulfonate. In certain embodiments, at least one R²¹ substituent is amino or substituted amino. In certain embodiments, at least one R²¹ substituent is amide. In certain embodiments, at least one R²¹ substituent is aryl or substituted aryl. In certain embodiments, at least one R²¹ substituent is heteroaryl or substituted heteroaryl. In certain embodiments, at least one R²¹ substituent is heterocycle or substituted heterocycle.

In some embodiments, the fragment (F1) is of the formula (F1A):

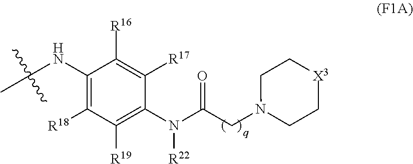

wherein:
q is an integer from 1 to 20;
R²² is selected from H, alkyl, substituted alkyl, aryl, substituted aryl, heterocycle, substituted heterocycle, heteroaryl, substituted heteroaryl;
R¹⁶, R¹⁷, R¹⁸ and R¹⁹ are each independently selected from H, alkyl, substituted alkyl, alkoxy, substituted alkoxy, halogen, CF³, sulfonate, amino, substituted amino, amide, aryl, substituted aryl, heteroaryl, substituted heteroaryl, heterocycle and substituted heterocycle; and X³ is selected from a heteroatom, a substituted heteroatom, CH₂ and a substituted carbon atom.

In certain embodiments of formula (F1A), X³ is a heteroatom or a substituted heteroatom. In certain cases, the heteroatom is O. In certain cases, the heteroatom is S. In certain other cases, X³ is NR²², wherein R²² is as described herein. In certain cases, R²² is H. In certain other cases, R²² is alkyl, such as methyl. In certain cases, X³ is CH₂ or a substituted carbon atom. In certain cases, X³ is a substituted carbon atom, wherein the substituent on the carbon atom is selected from alkyl, substituted alkyl, alkoxy, substituted alkoxy, halogen, CF₃, sulfonate, amino, substituted amino, amide, aryl, substituted aryl, heteroaryl, substituted heteroaryl, heterocycle and substituted heterocycle.

In certain embodiments of formula (F1A), q is 1. In certain embodiments, q is more than 1, such as 2, 3, 4, 5, 6, 7, 8, 9, 10, 11, 12, 13, 14, 15, 16, 17, 18, 19 or 20.

In certain embodiments of formula (F1A), R²² is H. In certain cases, R²² is alkyl or substituted alkyl. In certain cases, the alkyl group is a methyl group. In certain cases, R²² is aryl or substituted aryl. In certain cases, R²² is a heterocycle or substituted heterocycle. In certain cases, R²² is heteroaryl or substituted heteroaryl.

In certain embodiments of formula (F1A), each of R¹⁶, R¹⁷, R¹⁸ and R¹⁹ are H. In certain cases, at least one of R¹⁶, R¹⁷, R¹⁸ and R¹⁹ is a substituent selected from alkyl, substituted alkyl, alkoxy, substituted alkoxy, halogen, CF³, sulfonate, amino, substituted amino, amide, aryl, substituted aryl, heteroaryl, substituted heteroaryl, heterocycle and substituted heterocycle.

In certain embodiments of formula (F1A), at least one of R¹⁶, R¹⁷, R¹⁸ and R¹⁹ is alkyl or substituted alkyl. In certain embodiments, at least one of R¹⁶, R¹⁷, R¹⁸ and R¹⁹ is alkoxy or substituted alkoxy. In certain embodiments, at least one of R¹⁶, R¹⁷, R¹⁸ and R¹⁹ is halogen. In certain embodiments, at least one of R¹⁶, R¹⁷, R¹⁸ and R¹⁹ is CF₃. In certain embodiments, at least one of R¹⁶, R¹⁷, R¹⁸ and R¹⁹ is sulfonate. In certain embodiments, at least one of R¹⁶, R¹⁷, R¹⁸ and R¹⁹ is amino or substituted amino. In certain embodiments, at least one of R¹⁶, R¹⁷, R¹⁸ and R¹⁹ is amide. In certain embodiments, at least one of R¹⁶, R¹⁷, R¹⁸ and R¹⁹ is aryl or substituted aryl. In certain embodiments, at least one of R¹⁶, R¹⁷, R¹⁸ and R¹⁹ is heteroaryl or substituted heteroaryl. In certain embodiments, at least one of R¹⁶, R¹⁷, R¹⁸ and R¹⁹ is heterocycle or substituted heterocycle.

In certain embodiments, the formula (F1A) is of the structure:

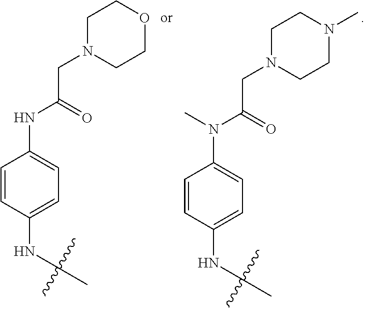

In some embodiments, the fragment (F1) is of the formula (F1B):

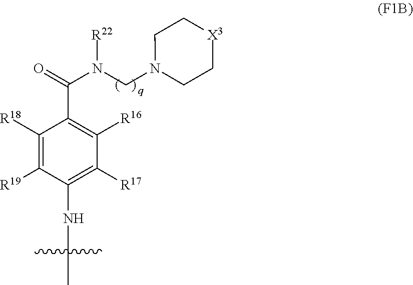

wherein:
q is an integer from 1 to 20;
R²² is selected from H, alkyl, substituted alkyl, aryl, substituted aryl, heterocycle, substituted heterocycle, heteroaryl, substituted heteroaryl;
R¹⁶, R¹⁷, R¹⁸ and R¹⁹ are each independently selected from H, alkyl, substituted alkyl, alkoxy, substituted alkoxy, halogen, CF³, sulfonate, amino, substituted amino, amide, aryl, substituted aryl, heteroaryl, substituted heteroaryl, heterocycle and substituted heterocycle; and
X³ is selected from a heteroatom, a substituted heteroatom, CH₂ and a substituted carbon atom.

In certain embodiments of formula (F1B), X³ is a heteroatom or a substituted heteroatom. In certain cases, the heteroatom is O. In certain cases, the heteroatom is S. In certain other cases, X³ is NR²², wherein R²² is as described herein. In certain cases, R²² is H. In certain other cases, R²² is alkyl, such as methyl. In certain cases, X³ is CH₂ or a substituted carbon atom. In certain cases, X³ is a substituted carbon atom, wherein the substituent on the carbon atom is selected from alkyl, substituted alkyl, alkoxy, substituted alkoxy, halogen, CF₃, sulfonate, amino, substituted amino, amide, aryl, substituted aryl, heteroaryl, substituted heteroaryl, heterocycle and substituted heterocycle.

In certain embodiments of formula (F1B), q is 1. In certain embodiments, q is more than 1, such as 2, 3, 4, 5, 6, 7, 8, 9, 10, 11, 12, 13, 14, 15, 16, 17, 18, 19 or 20.

In certain embodiments of formula (F1B), R²² is H. In certain cases, R²² is alkyl or substituted alkyl. In certain cases, the alkyl group is a methyl group. In certain cases, R²² is aryl or substituted aryl. In certain cases, R²² is a heterocycle or substituted heterocycle. In certain cases, R²² is heteroaryl or substituted heteroaryl.

In certain embodiments of formula (F1B), each of R¹⁶, R¹⁷, R¹⁸ and R¹⁹ are H. In certain cases, at least one of R¹⁶, R¹⁷, R¹⁸ and R¹⁹ is a substituent selected from alkyl, substituted alkyl, alkoxy, substituted alkoxy, halogen, CF³, sulfonate, amino, substituted amino, amide, aryl, substituted aryl, heteroaryl, substituted heteroaryl, heterocycle and substituted heterocycle.

In certain embodiments of formula (F1B), at least one of R¹⁶, R¹⁷, R¹⁸ and R¹⁹ is alkyl or substituted alkyl. In certain embodiments, at least one of R¹⁶, R¹⁷, R¹⁸ and R¹⁹ is alkoxy or substituted alkoxy. In certain embodiments, at least one of R¹⁶, R¹⁷, R¹⁸ and R¹⁹ is halogen. In certain embodiments, at least one of R¹⁶, R¹⁷, R¹⁸ and R¹⁹ is CF₃. In certain embodiments, at least one of R¹⁶, R¹⁷, R¹⁸ and R¹⁹ is sulfonate. In certain embodiments, at least one of R¹⁶, R¹⁷, R¹⁸ and R¹⁹ is amino or substituted amino. In certain embodiments, at least one of R¹⁶, R¹⁷, R¹⁸ and R¹⁹ is amide. In certain embodiments, at least one of R¹⁶, R¹⁷, R¹⁸ and R¹⁹ is aryl or substituted aryl. In certain embodiments, at least one of R¹⁶, R¹⁷, R¹⁸ and R¹⁹ is heteroaryl or substituted heteroaryl. In certain embodiments, at least one of R¹⁶, R¹⁷, R¹⁸ and R¹⁹ is heterocycle or substituted heterocycle.

In certain embodiments, the formula (F1B) is of the structure:

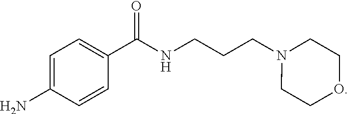 [SMILES: Nc1ccc(C(=O)NCCCN2CCOCC2)cc1]

In some embodiments, the fragment (F1) is of the formula (F1C):

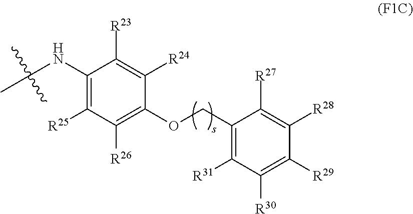

wherein:
s is an integer from 1 to 20; and
R²³ to R³¹ are each independently selected from H, alkyl, substituted alkyl, alkoxy, substituted alkoxy, halogen, CF₃, sulfonate, amino, substituted amino, amide, aryl, substituted aryl, heterocycle, substituted heterocycle, heterocycle and substituted heterocycle.

In certain embodiments of formula (F1C), s is 1. In certain embodiments, s is more than 1, such as 2, 3, 4, 5, 6, 7, 8, 9, 10, 11, 12, 13, 14, 15, 16, 17, 18, 19 or 20.

In certain embodiments of formula (F1B), each of R²³ to R³¹ are H. In certain cases, at least one of R²³ to R³¹ is a substituent selected from alkyl, substituted alkyl, alkoxy, substituted alkoxy, halogen, CF³, sulfonate, amino, substituted amino, amide, aryl, substituted aryl, heteroaryl, substituted heteroaryl, heterocycle and substituted heterocycle.

In certain embodiments of formula (F1C), at least one of R²³ to R³¹ is alkyl or substituted alkyl. In certain embodiments, at least one of R²³ to R³¹ is alkoxy or substituted alkoxy. In certain embodiments, at least one of R²³ to R³¹ is halogen. In certain embodiments, at least one of R²³ to R³¹ is CF₃. In certain embodiments, at least one of R²³ to R³¹ is sulfonate. In certain embodiments, at least one of R²³ to R³¹ is amino or substituted amino. In certain embodiments, at least one of R²³ to R³¹ is amide. In certain embodiments, at least one of R²³ to R³¹ is aryl or substituted aryl. In certain embodiments, at least one of R²³ to R³¹ is heteroaryl or substituted heteroaryl. In certain embodiments, at least one of R²³ to R³¹ is heterocycle or substituted heterocycle.

In certain embodiments, the formula (F1C) is of the structure:

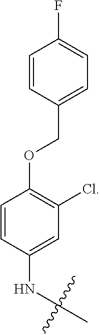 [SMILES: *Nc1ccc(OCc2ccc(F)cc2)c(Cl)c1]

In other embodiments, the formula (F1) is of the structure:

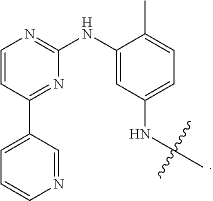 [SMILES: *Nc1ccc(C)c(Nc2nccc(-c3cccnc3)n2)c1]

In some embodiments of the subject functional dye, the small molecule that targets a protein (F) is of the formula (F2):

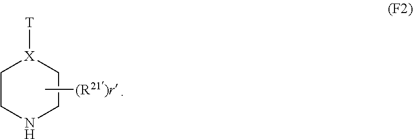

wherein
X is selected from a N, CH and CR³², wherein R³² is selected from alkyl, substituted alkyl, hydroxy, alkoxy, substituted alkoxy, halogen, CF₃, sulfonate, amino, substituted amino, amide, aryl, substituted aryl, heteroaryl, substituted heteroaryl, heterocycle and substituted heterocycle;
T is a protein targeting moiety comprising a fragment of a known drug that targets a protein selected from the group consisting of (non)-receptor kinases, immune checkpoint proteins, G-protein-coupled receptors (GPCRs), influx transporters, efflux transporters, ion channels, human leukocyte antigens (HLA), proteases, caspases and nuclear receptors from a small molecule kinase inhibitor or a fragment of a small molecule modulator of GPCRs;
R^{21′} are each independently selected from alkyl, substituted alkyl, alkoxy, substituted alkoxy, halogen, CF₃, sulfonate, amino, substituted amino, amide, aryl, substituted aryl, heteroaryl, substituted heteroaryl, heterocycle and substituted heterocycle; and
r′ is an integer from 0 to 8.

In certain embodiments of formula (F2), X is N. In certain cases, X is CH₂ or a substituted carbon atom. In certain cases, X is CR³², wherein R³² is selected from alkyl, substituted alkyl, hydroxy, alkoxy, substituted alkoxy, halogen, CF₃, sulfonate, amino, substituted amino, amide, aryl, substituted aryl, heteroaryl, substituted heteroaryl, heterocycle and substituted heterocycle. In certain cases, R³² is as defined for group T. In certain cases, X is CR³² and R³² is hydroxy.

In certain embodiments of formula (F2), T is a fragment of a small molecule kinase inhibitor. In certain cases, the small molecule kinase inhibitor is selected from imatinib, bosutinib, nintedanib, ponatinib, brigatinib and dasatinib, ceritinib, erlotinib and palbociclib.

In certain embodiments of formula (F2), T is a fragment of a small molecule modulator of G-protein-coupled receptors (GPCRs). In certain cases, the small molecule modulator of GPCRs is selected from sildenafil and eszopiclone.

In certain embodiments of formula (F2), T is a fragment of a small molecule androgen receptor inhibitor. In some cases, the androgen receptor inhibitor is enzalutamide.

In certain embodiments of formula (F2), T is a fragment of a small molecule a programmed cell death ligand 1 inhibitor (PD-L1 inhibitor). In some cases, the PD-L1 inhibitor is a compound of structure (9), or a fragment thereof.

In certain embodiments of formula (F2), r′ is 0 such that the compound of formula (F2) contains no R^{21′} substituents. In other cases, r′ is 1 such that the compound of formula (F2) contains one R^{21′} substituent. In other cases, r′ is 2 such that the compound of formula (F2) contains two R^{21′} substituents. In other cases, r′ is 3 such that the compound of formula (F2) contains three R^{21′} substituents. In other cases, r′ is 4 such that the compound of formula (F2) contains four R^{21′} substituents. In other cases, r′ is 5 such that the compound of formula (F2) contains five R^{21′} substituents. In other cases, r′ is 6 such that the compound of formula (F2) contains six R^{21′} substituents. In other cases, r′ is 7 such that the compound of formula (F2) contains seven R^{21′} substituents. In other cases, r′ is 8 such that the compound of formula (F2) contains eight R^{21′} substituents.

In certain embodiments of formula (F2), at least one R^{21′} substituent is alkyl or substituted alkyl. In certain embodiments, at least one R^{21′} substituent is alkoxy or substituted alkoxy. In certain embodiments, at least one R^{21′} substituent is halogen. In certain embodiments, at least one R^{21′} substituent is CF₃. In certain embodiments, at least one R^{21′} substituent is sulfonate. In certain embodiments, at least one R^{21′} substituent is amino or substituted amino. In certain embodiments, at least one R^{21′} substituent is amide. In certain embodiments, at least one R^{21′} substituent is aryl or substituted aryl. In certain embodiments, at least one R^{21′} substituent is heteroaryl or substituted heteroaryl. In certain embodiments, at least one R^{21′} substituent is heterocycle or substituted heterocycle.

In some cases, the subject functional dye is of the formula (I):

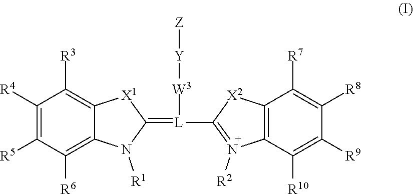

wherein:
L is a polymethine group or a substituted polymethine group;
R¹ and R² are each independently selected from H, alkyl, and (CH₂)ₙR¹², wherein n is an integer from 1 to 20 and R¹² is selected from the group consisting of sulfonate, carboxylic acid, amine, quaternary ammonium cation, phosphate, ester, halogen, azide, cyano, alkyne, and heterocycle;
R³ and R⁴, are each independently selected from H, alkyl, substituted alkyl, alkoxy, substituted alkoxy, halogen, sulfonate, amino, substituted amino, amide, aryl, substituted aryl, heterocycle and substituted heterocycle;
or R³ and R⁴ together with the carbon to which they are attached form a group selected from aryl, substituted aryl, heterocycle, substituted heterocycle, cycloalkyl and substituted cycloalkyl;
R⁷ and R⁸ are each independently selected from H, alkyl, substituted alkyl, alkoxy, substituted alkoxy, halogen, sulfonate, amino, substituted amino, amide, aryl, substituted aryl, heterocycle and substituted heterocycle;
or R⁷ and R⁸ together with the carbon to which they are attached form a group selected from aryl, substituted aryl, heterocycle, substituted heterocycle, cycloalkyl and substituted cycloalkyl;
R⁵, R⁶, R⁹ and R¹⁰ are each independently selected from H, alkyl, substituted alkyl, alkoxy, substituted alkoxy, halogen, sulfonate, amino, substituted amino, amide, aryl, substituted aryl, heterocycle and substituted heterocycle;
X¹ and X² are each independently selected from a heteroatom, CH₂, C(R¹³)₂, wherein each R¹³ is independently selected from H, alkyl and substituted alkyl;
W¹ is selected from a heteroatom or C;
W² is selected from a cationic heteroatom;
W³ is selected from a heteroatom, NR¹¹ or C(R¹¹)₂, wherein R¹¹ is selected from H or alkyl;
Y is selected from the group consisting of alkyl, substituted alkyl, aryl, substituted aryl, carboxyl and substituted carboxyl; and
Z is selected from the group consisting of substituted amine, morpholine, substituted morpholine, thiomorpholine, substituted thiomorpholine, piperazine and substituted piperazine; or a (pro-)drug, a pharmaceutically acceptable salt or a solvate thereof.

In some embodiments of formula (I), the structure of the functional dye has the formula (IA):

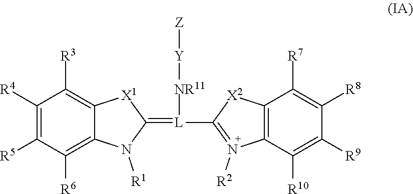

wherein:
L is a polymethine group or a substituted polymethine group;
R¹ and R² are each independently selected from H, alkyl, and (CH₂)ₙR¹², wherein n is an integer from 1 to 20 and R¹² is selected from the group consisting of sulfonate, carboxylic acid, amine, quaternary ammonium cation, phosphate, ester, halogen, azide, cyano, alkyne, and heterocycle;
R³ and R⁴, are each independently selected from H, alkyl, substituted alkyl, alkoxy, substituted alkoxy, halogen, sulfonate, amino, substituted amino, amide, aryl, substituted aryl, heterocycle and substituted heterocycle;
or R³ and R⁴ together with the carbon to which they are attached form a group selected from aryl, substituted aryl, heterocycle, substituted heterocycle, cycloalkyl and substituted cycloalkyl;
R⁷ and R⁸ are each independently selected from H, alkyl, substituted alkyl, alkoxy, substituted alkoxy, halogen, sulfonate, amino, substituted amino, amide, aryl, substituted aryl, heterocycle and substituted heterocycle;
or R⁷ and R⁸ together with the carbon to which they are attached form a group selected from aryl, substituted aryl, heterocycle, substituted heterocycle, cycloalkyl and substituted cycloalkyl;
R⁵, R⁶, R⁹ and R¹⁰ are each independently selected from H, alkyl, substituted alkyl, alkoxy, substituted alkoxy, halogen, sulfonate, amino, substituted amino, amide, aryl, substituted aryl, heterocycle and substituted heterocycle;
X¹ and X² are each independently selected from a heteroatom, CH₂, C(R¹³)₂, wherein each R¹³ is independently selected from H, alkyl and substituted alkyl;
R¹¹ is selected from H or alkyl;
Y is selected from the group consisting of alkyl, substituted alkyl, aryl, substituted aryl, carboxyl and substituted carboxyl; and
Z is selected from the group consisting of substituted amine, morpholine, substituted morpholine, thiomorpholine, substituted thiomorpholine, piperazine and substituted piperazine; or a (pro-)drug, a pharmaceutically acceptable salt or a solvate thereof.

In some embodiments of formula (I), L is selected from one of the following polymethine groups:

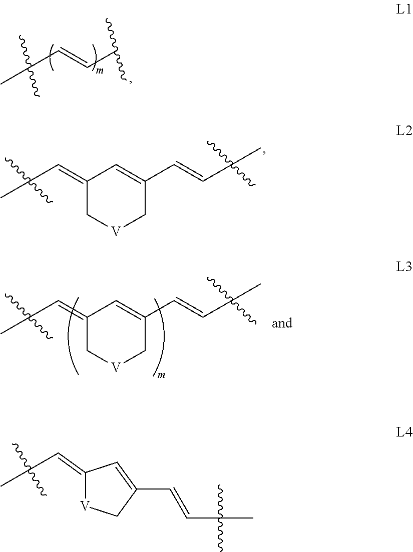

wherein, m is an integer from 1 to 4; and
V is selected from a heteroatom, C(R²¹)₂, wherein each R²¹ is independently selected from H or an alkyl group. In some cases of any of L2-L4, V is C(R²¹)₂ and R²¹ is H. In some cases, V is C(R²¹)₂ and R²¹ is an alkyl group. In certain cases V is a heteroatom, such as O, S, N, Se, Te. In some cases of any of L2-L4, V is O. In some cases of any of L2-L4, V is S. In some cases of any of L2-L4, V is N. In some cases of any of L2-L4, V is Se. In some cases of any of L2-L4, V is Te. In certain cases of L1 or L3, m is the integer 4. In some cases, m is an integer less than 4, such as 3, 2 or 1.

In certain instances of formula (I), L is:

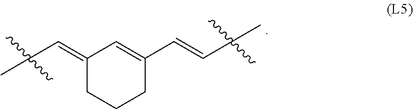 [SMILES: */C=C/C1=CC(=C/*)/CCC1]

In some embodiments of formula (I), the structure of the functional dye has the formula (II):

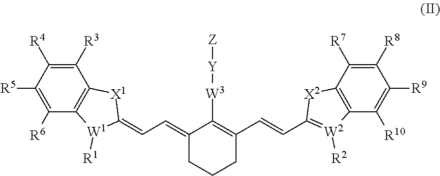

wherein:
R¹ and R² are each independently selected from H, alkyl, and (CH₂)ₙR¹², wherein n is an integer from 1 to 20 and R¹² is selected from the group consisting of sulfonate, carboxylic acid, amine, quaternary ammonium cation, phosphate, ester, halogen, azide, cyano, alkyne, and heterocycle;
R³ and R⁴, are each independently selected from H, alkyl, substituted alkyl, alkoxy, substituted alkoxy, halogen, sulfonate, amino, substituted amino, amide, aryl, substituted aryl, heterocycle and substituted heterocycle;
or R³ and R⁴ together with the carbon to which they are attached form a group selected from aryl, substituted aryl, heterocycle, substituted heterocycle, cycloalkyl and substituted cycloalkyl;
R⁷ and R⁸ are each independently selected from H, alkyl, substituted alkyl, alkoxy, substituted alkoxy, halogen, sulfonate, amino, substituted amino, amide, aryl, substituted aryl, heterocycle and substituted heterocycle;
or R⁷ and R⁸ together with the carbon to which they are attached form a group selected from aryl, substituted aryl, heterocycle, substituted heterocycle, cycloalkyl and substituted cycloalkyl;
R⁵, R⁶, R⁹ and R¹⁰ are each independently selected from H, alkyl, substituted alkyl, alkoxy, substituted alkoxy, halogen, sulfonate, amino, substituted amino, amide, aryl, substituted aryl, heterocycle and substituted heterocycle;
X¹ and X² are each independently selected from a heteroatom, CH₂, C(R¹³)₂, wherein each R¹³ is independently selected from H, alkyl and substituted alkyl;
W¹ is selected from a heteroatom or CH₂;
W² is selected from a cationic heteroatom;
W³ is selected from a heteroatom, NR¹¹ or C(R¹¹)₂, wherein R¹¹ is selected from H or alkyl;
Y is selected from the group consisting of alkyl, substituted alkyl, aryl, substituted aryl, carboxyl and substituted carboxyl; and
Z is selected from the group consisting of substituted amine, morpholine, substituted morpholine, thiomorpholine, substituted thiomorpholine, piperazine and substituted piperazine; or a pro-drug, a pharmaceutically acceptable salt or a solvate thereof.

In some embodiments of formula (II), the functional dye has the structure (IIA):

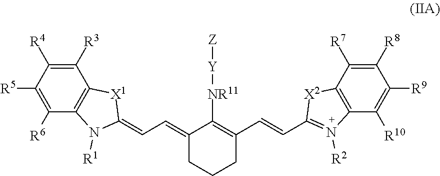

wherein:
R¹ and R² are each independently selected from H, alkyl, and (CH₂)ₙR¹², wherein n is an integer from 1 to 20 and R¹² is selected from the group consisting of sulfonate, carboxylic acid, amine, quaternary ammonium cation, phosphate, ester, halogen, azide, cyano, alkyne, and heterocycle;
R³ and R⁴, are each independently selected from H, alkyl, substituted alkyl, alkoxy, substituted alkoxy, halogen, sulfonate, amino, substituted amino, amide, aryl, substituted aryl, heterocycle and substituted heterocycle;
or R³ and R⁴ together with the carbon to which they are attached form a group selected from aryl, substituted aryl, heterocycle, substituted heterocycle, cycloalkyl and substituted cycloalkyl;
R⁷ and R⁸ are each independently selected from H, alkyl, substituted alkyl, alkoxy, substituted alkoxy, halogen, sulfonate, amino, substituted amino, amide, aryl, substituted aryl, heterocycle and substituted heterocycle;
or R⁷ and R⁸ together with the carbon to which they are attached form a group selected from aryl, substituted aryl, heterocycle, substituted heterocycle, cycloalkyl and substituted cycloalkyl;
R⁵, R⁶, R⁹ and R¹⁰ are each independently selected from H, alkyl, substituted alkyl, alkoxy, substituted alkoxy, halogen, sulfonate, amino, substituted amino, amide, aryl, substituted aryl, heterocycle and substituted heterocycle;
X¹ and X² are each independently selected from a heteroatom, CH₂, C(R¹³)₂, wherein each R¹³ is independently selected from H, alkyl and substituted alkyl;
R¹¹ is selected from H or alkyl;
Y is selected from the group consisting of alkyl, substituted alkyl, aryl, substituted aryl, carboxyl and substituted carboxyl; and
Z is selected from the group consisting of substituted amine, morpholine, substituted morpholine, thiomorpholine, substituted thiomorpholine, piperazine and substituted piperazine; or a pro-drug, a pharmaceutically acceptable salt or a solvate thereof.

In some embodiments of formula (I) or formula (II), W¹ and W² are each independently selected from the heteroatoms O, S, N, Se, Te. In certain cases W¹ and W² are the same heteroatom. In other cases, W¹ and W² are different heteroatoms. In some cases, W¹ and W² are both N. In some cases, W¹ and W² are both O. In some cases W¹ and W² are both S. In some cases W¹ and W² are both Se. In some cases W¹ and W² are both Te. In some cases, W¹ and W² are both C. In certain instances W¹ is a C atom and W² is a heteroatom (e.g. O, S, N, Se, Te). In other instances one of W² is a C atom and W¹ is a heteroatom (e.g. O, S, N, Se, Te).

In some embodiments of formula (I) or formula (II), R¹ and R² are both (CH₂)ₙR¹² and each R¹² is independently selected from the group consisting of sulfonate, carboxylic acid, amine, quaternary ammonium cation, phosphate, ester, halogen, azide, cyano, alkyne, and heterocycle. In certain cases, each R¹² group are the same. In other cases, each R¹² group are different. In certain cases, at least one R¹² group is a sulfonate or a salt thereof. In some cases, both R¹² groups are a sulfonate or a salt thereof. In certain cases n is an integer less than 20, such as 15 or less, 10 or less, 5 or less, or even less. In some instances, n is an integer less than 10, such as 9, 8, 7, 6, 5, 4, 3, 2 or 1. In certain instances, n is 4. In certain instances n is 3.

In certain cases of formula (I) or formula (II), R¹ and R² are both (CH₂)ₙR¹², wherein n is 4 and R¹² is sulfonate or a salt thereof.

In some embodiments of formula (I) or formula (II), X¹ and X² are each independently selected from the heteroatoms O, S, N, Se, CH₂ or C(R¹³)₂ wherein each R¹³ is independently selected from H, alkyl and substituted alkyl. In certain cases one or both of X¹ and X² are heteroatoms. In other cases, X¹ and X² are each different heteroatoms. In other cases X¹ and X² are each the same heteroatoms. In certain cases X¹ and X² are each independently selected from CH₂ and C(R¹³)₂ wherein each R¹³ is independently selected from H, alkyl and substituted alkyl. In some cases, X¹ and X² are the same group. In some cases, X¹ and X² are different groups. In some cases X¹ and X² are both CH₂. In some cases X¹ and X² are both C(R¹³)₂ wherein each R¹³ is independently selected from H, alkyl and substituted alkyl.

In some cases of formula (I) or formula (II), X¹ and X² are both C(R¹³)₂, wherein R¹³ is methyl.

In some embodiments of formula (I) or formula (II), each of R³ to R¹⁰ is H. In some embodiments of formula (I) or formula (II), one or more of R³ to R¹⁰ is a substitute other than H.

In some embodiments of formula (I) or formula (II), W³ is selected from a heteroatom (e.g. O, S, N, Se, Te), NR¹¹ or C(R¹¹)₂, wherein R¹¹ is selected from H or alkyl. In certain cases W³ is a heteroatom selected from O, S, N, Se or Te. In certain cases W³ is NR¹¹. In some cases when W³ is NR¹¹, R¹¹ is H. In other cases, R¹¹ is an alkyl group.

In certain embodiments of formula (II), the structure of the functional dye has the formula (III):

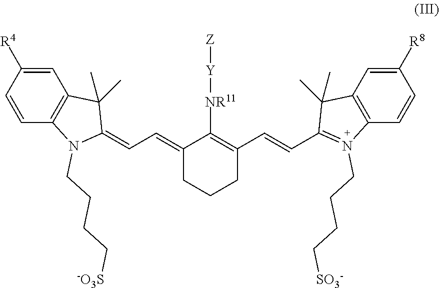

wherein:
R4 and R8 are each independently selected from H, alkyl, substituted alkyl, alkoxy, substituted alkoxy, halogen, sulfonate, amino, substituted amino, amide, aryl, substituted aryl, heterocycle and substituted heterocycle;
R¹¹ is selected from H or alkyl;
Y is selected from the group consisting of alkyl, substituted alkyl, aryl, substituted aryl, carboxyl and substituted carboxyl;
Z is selected from the group consisting of substituted amine, morpholine, substituted morpholine, thiomorpholine, substituted thiomorpholine, piperazine and substituted piperazine; or a pro-drug, a pharmaceutically acceptable salt or a solvate thereof.

In some embodiments of any one of formulas (I)-(III), the group Y is selected from:

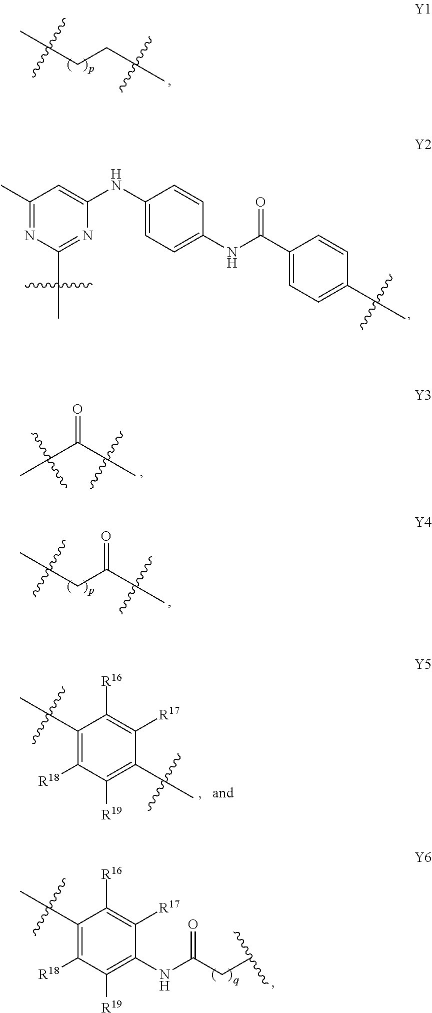

wherein:
each p is independently an integer from 1 to 20;
q is an integer from 1 to 20;
R¹⁶, R¹⁷, R¹⁸ and R¹⁹ are each independently selected from H, alkyl, substituted alkyl, alkoxy, substituted alkoxy, halogen, sulfonate, amino, substituted amino, amide, aryl, substituted aryl, heterocycle and substituted heterocycle.

In some cases of Y1 to Y4, p is an integer less than 20, such as 15 or less, 10 or less, 5 or less, or even less. In some instances, p is an integer less than 5, such as 4, 3, 2, or 1. In some cases of Y6, q is an integer less than 20, such as 15 or less, 10 or less, 5 or less, or even less. In some instances, q is an integer less than 5, such as 4, 3, 2, or 1. In certain instances of Y5 or Y6, each of R¹⁶, R¹⁷, R¹⁸ and R¹⁹ is H. In other cases, at least one of R¹⁶, R¹⁷, R¹⁸ and R¹⁹ is a substitute other than H.

In some embodiments of any one of formulas (I)-(III), the group Z is selected from:

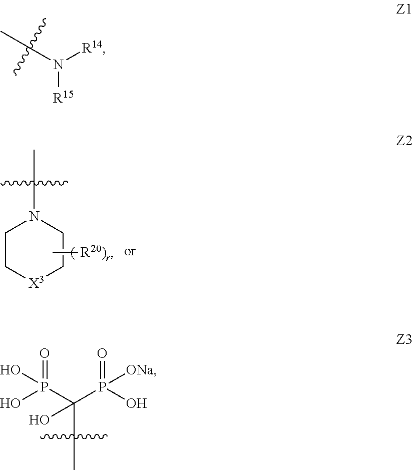

wherein:
R¹⁴ and R¹⁵ are each independently selected from C₁₋₆ alkyl;
R²⁰ is a substituent selected from alkyl, substituted alkyl, alkoxy, substituted alkoxy, halogen, sulfonate, amino, substituted amino, amide, aryl, substituted aryl, heterocycle and substituted heterocycle;
r is an integer from 0 to 4; and
X³ is selected from O, S, N and C.

In certain cases of a formula of Z1, R¹⁴ and R¹⁵ are each independently selected from a lower alkyl group, such as hexyl, pentyl, butyl, propyl, ethyl or methyl. In certain cases of formula Z1, R¹⁴ and R¹⁵ are different lower alkyl groups. In other cases of formula Z1, R¹⁴ and R¹⁵ are the same lower alkyl groups. In some cases, both of R¹⁴ and R¹⁵ are ethyl groups.

In certain cases of a formula of Z2, X³ is a heteroatom selected from O, S or N. In certain cases X³ is O. In certain cases X³ is S. In certain other cases X³ is N. In some cases, X³ is a carbon atom. In certain cases of a formula of Z2, r is an integer less than 4, such as 3, 2, 1 or 0. In some cases, r is 0 and the formula of Z2 contains no R²⁰ substituents.

In certain instances of any one of formulas (I)-(III), Z is Z2, and Z2 is a morpholine group or a substituted morpholine group. In certain cases of formulas (I)-(III), Y is Y4. In other cases, Y is Y6.

In certain instances of anyone of formulas (I)-(III) NR¹¹—Y—Z together forma group selected from:

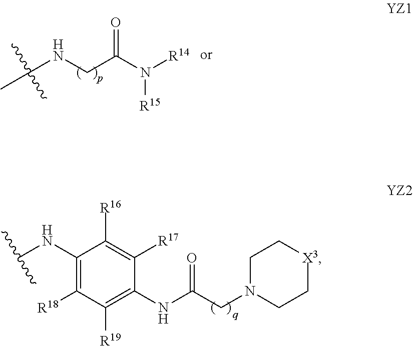

wherein:

p is an integer from 0 to 20;

R¹⁴ and R¹⁵ are each independently selected from C₁₋₆ alkyl;

q is an integer from 1 to 20;

R¹⁶, R¹⁷, R¹⁸ and R¹⁹ are each independently selected from H, alkyl, substituted alkyl, alkoxy, substituted alkoxy, halogen, sulfonate, amino, substituted amino, amide, aryl, substituted aryl, heterocycle and substituted heterocycle; and

X³ is selected from O, S, N and C.

In certain cases of a formula of YZ1, R¹⁴ and R¹⁵ are each independently selected from a lower alkyl group, such as hexyl, pentyl, butyl, propyl, ethyl or methyl. In certain cases of formula YZ1, R¹⁴ and R¹⁵ are different lower alkyl groups. In other cases of formula YZ1, R¹⁴ and R¹⁵ are the same lower alkyl groups. In some cases, both of R¹⁴ and R¹⁵ are ethyl groups. In some cases of a formula of YZ1, p is an integer less than 20, such as 15 or less, 10 or less, 5 or less, or even less. In some instances, p is an integer less than 5, such as 4, 3, 2, or 1.

In some cases of YZ2, q is an integer less than 20, such as 15 or less, 10 or less, 5 or less, or even less. In some instances, q is an integer less than 5, such as 4, 3, 2, or 1. In certain instances of YZ2, each of R¹⁶, R¹⁷, R¹⁸ and R¹⁹ is H. In other cases, at least one of R¹⁶, R¹⁷, R¹⁸ and R¹⁹ is a substitute other than H. In certain cases of a formula of YZ2, X³ is a heteroatom selected from O, S or N. In certain cases X³ is O. In certain cases X³ is S. In certain other cases X is N. In some cases, X³ is a carbon atom.

In certain instances of anyone of formulas (I)-(III) NR¹¹—Y—Z together form:

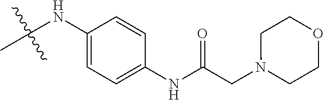 [SMILES: *Nc1ccc(NC(=O)CN2CCOCC2)cc1]

In certain embodiments of any one of formulas (I)-(III) and, the functional dye is described by the structures 1-4:

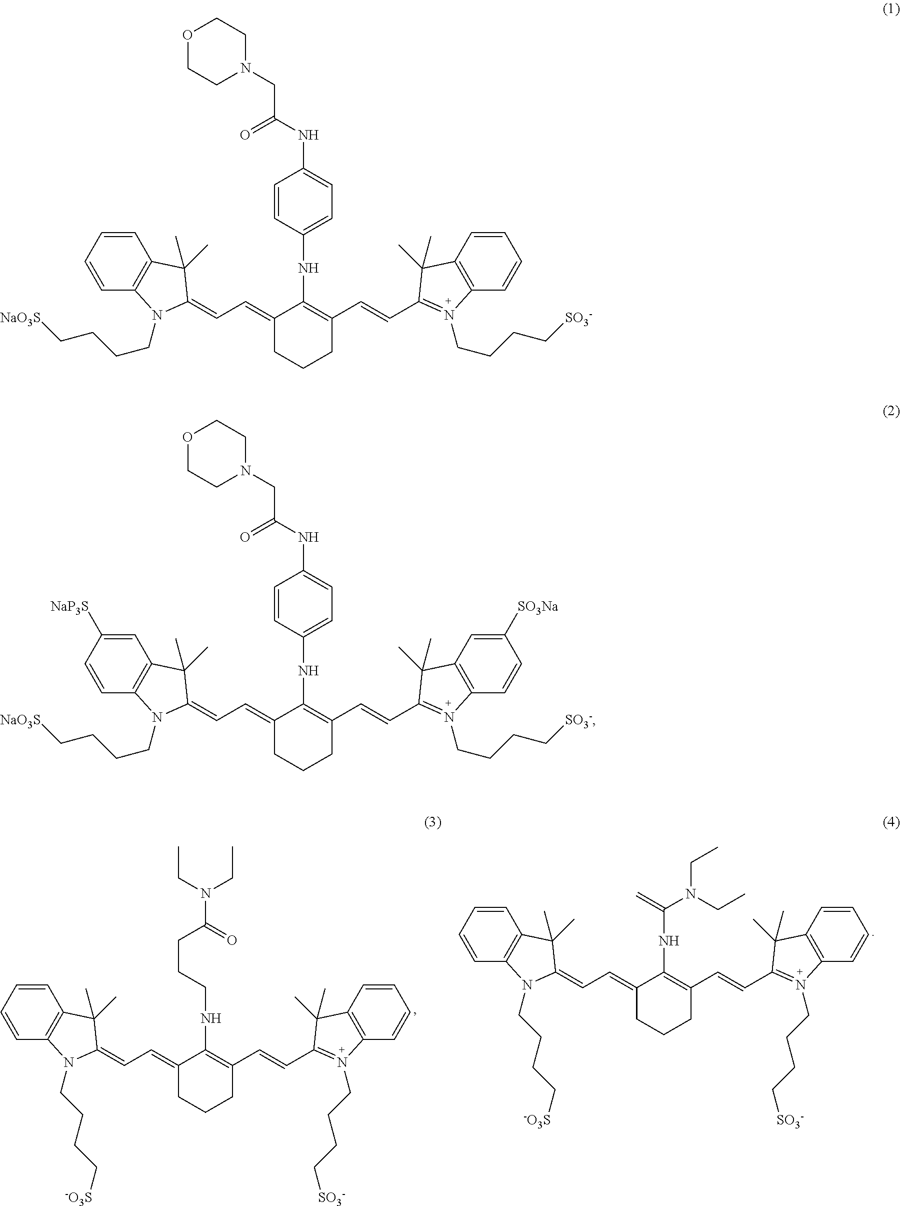

In certain embodiments, the subject functional dye is of the formula (A1):

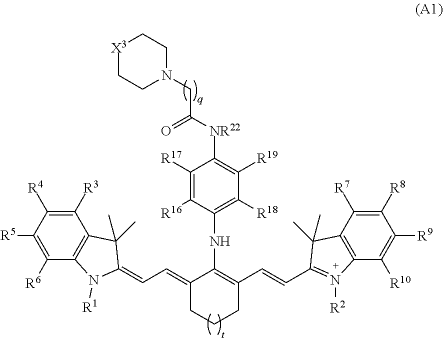

wherein:
R¹ and R² are each independently selected from H, alkyl, and (CH₂)ₙR¹², wherein n is an integer from 1 to 20 and R¹² is selected from the group consisting of H, sulfonate, carboxylic acid, amine, quaternary ammonium cation, phosphate, ester, halogen, azide, cyano, alkyne, and heterocycle;
R³ and R⁴, are each independently selected from H, alkyl, substituted alkyl, alkoxy, substituted alkoxy, halogen, CF₃, carboxy, phosphonate, carboxylate, cyano, hydroxyl, carboxyamide, sulfonamide, sulfonate, amino, substituted amino, amide, aryl, substituted aryl, heteroaryl, substituted heteroaryl, heterocycle and substituted heterocycle;
or R³ and R⁴ together with the carbon to which they are attached form a group selected from aryl, substituted aryl, heteroaryl, substituted heteroaryl, heterocycle, substituted heterocycle, cycloalkyl and substituted cycloalkyl;
R⁷ and R⁸ are each independently selected from H, alkyl, substituted alkyl, alkoxy, substituted alkoxy, halogen, CF₃, carboxy, phosphonate, carboxylate, cyano, hydroxyl, carboxyamide, sulfonamide, sulfonate, amino, substituted amino, amide, aryl, substituted aryl, heteroaryl, substituted heteroaryl, heterocycle and substituted heterocycle;
or R⁷ and R⁸ together with the carbon to which they are attached form a group selected from aryl, substituted aryl, heteroaryl, substituted heteroaryl, heterocycle, substituted heterocycle, cycloalkyl and substituted cycloalkyl; and
R⁵, R⁶, R⁹ and R¹⁰ are each independently selected from H, alkyl, substituted alkyl, alkoxy, substituted alkoxy, halogen, CF₃, carboxy, phosphonate, carboxylate, cyano, hydroxyl, carboxyamide, sulfonamide, sulfonate, amino, substituted amino, amide, aryl, substituted aryl, heteroaryl, substituted heteroaryl, heterocycle and substituted heterocycle;
t is an integer from 0 to 1;
q is an integer from 1 to 20;
R²² is selected from H, alkyl, substituted alkyl, aryl, substituted aryl, heterocycle, substituted heterocycle, heteroaryl, substituted heteroaryl;
R¹⁶, R¹⁷, R¹⁸ and R¹⁹ are each independently selected from H, alkyl, substituted alkyl, alkoxy, substituted alkoxy, halogen, CF₃, sulfonate, amino, substituted amino, amide, aryl, substituted aryl, heteroaryl, substituted heteroaryl, heterocycle and substituted heterocycle; and
X³ is selected from a heteroatom, a substituted heteroatom, CH₂ and a substituted carbon atom.

In certain embodiments, the subject functional dye is of the formula (A1a):

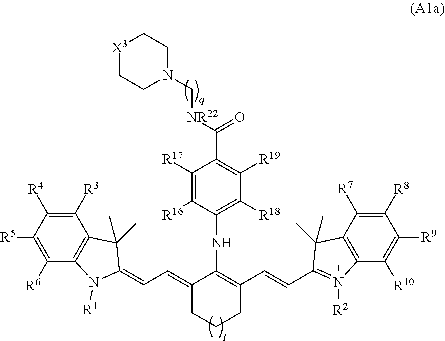

wherein:
R¹ and R² are each independently selected from H, alkyl, and (CH₂)ₙR¹², wherein n is an integer from 1 to 20 and R¹² is selected from the group consisting of H, sulfonate, carboxylic acid, amine, quaternary ammonium cation, phosphate, ester, halogen, azide, cyano, alkyne, and heterocycle;
R³ and R⁴, are each independently selected from H, alkyl, substituted alkyl, alkoxy, substituted alkoxy, halogen, CF₃, carboxy, phosphonate, carboxylate, cyano, hydroxyl, carboxyamide, sulfonamide, sulfonate, amino, substituted amino, amide, aryl, substituted aryl, heteroaryl, substituted heteroaryl, heterocycle and substituted heterocycle;
or R³ and R⁴ together with the carbon to which they are attached form a group selected from aryl, substituted aryl, heteroaryl, substituted heteroaryl, heterocycle, substituted heterocycle, cycloalkyl and substituted cycloalkyl;
R⁷ and R⁸ are each independently selected from H, alkyl, substituted alkyl, alkoxy, substituted alkoxy, halogen, CF₃, carboxy, phosphonate, carboxylate, cyano, hydroxyl, carboxyamide, sulfonamide, sulfonate, amino, substituted amino, amide, aryl, substituted aryl, heteroaryl, substituted heteroaryl, heterocycle and substituted heterocycle;
or R⁷ and R⁸ together with the carbon to which they are attached form a group selected from aryl, substituted aryl, heteroaryl, substituted heteroaryl, heterocycle, substituted heterocycle, cycloalkyl and substituted cycloalkyl; and
R⁵, R⁶, R⁹ and R¹⁰ are each independently selected from H, alkyl, substituted alkyl, alkoxy, substituted alkoxy, halogen, CF₃, carboxy, phosphonate, carboxylate, cyano, hydroxyl, carboxyamide, sulfonamide, sulfonate, amino, substituted amino, amide, aryl, substituted aryl, heteroaryl, substituted heteroaryl, heterocycle and substituted heterocycle;
t is an integer from 0 to 1;
q is an integer from 1 to 20;
R²² is selected from H, alkyl, substituted alkyl, aryl, substituted aryl, heterocycle, substituted heterocycle, heteroaryl, substituted heteroaryl;
R¹⁶, R¹⁷, R¹⁸ and R¹⁹ are each independently selected from H, alkyl, substituted alkyl, alkoxy, substituted alkoxy, halogen, CF₃, sulfonate, amino, substituted amino, amide, aryl, substituted aryl, heteroaryl, substituted heteroaryl, heterocycle and substituted heterocycle; and
X³ is selected from a heteroatom, a substituted heteroatom, CH₂ and a substituted carbon atom.

In some embodiments of formula (A1) or (A1a), R¹ and R² are both (CH₂)ₙR¹² and each R¹² is independently selected from the group consisting of sulfonate, carboxylic acid, amine, quaternary ammonium cation, phosphate, ester, halogen, azide, cyano, alkyne, and heterocycle. In certain cases, each R¹² group are the same. In other cases, each R¹² group are different. In certain cases, at least one R¹² group is a sulfonate or a salt thereof. In some cases, both R¹² groups are a sulfonate or a salt thereof. In certain cases, n is an integer less than 20, such as 15 or less, 10 or less, 5 or less, or even less. In some instances, n is an integer less than 10, such as 9, 8, 7, 6, 5, 4, 3, 2 or 1. In certain instances, n is 4. In certain instances, n is 3.

In certain cases of formula (A1), R¹ and R² are both (CH₂)ₙR¹², wherein n is 4 and R¹² is sulfonate or a salt thereof.

In certain embodiments of formula (A1) or (A1a), each of R³ to R¹⁰ are H. In certain cases, at least one of R³ to R¹⁰ is a substituent selected from alkyl, substituted alkyl, alkoxy, substituted alkoxy, halogen, CF³, sulfonate, amino, substituted amino, amide, aryl, substituted aryl, heteroaryl, substituted heteroaryl, heterocycle and substituted heterocycle.

In certain embodiments of formula (A1) or (A1a), at least one of R³ to R¹⁰ is alkyl or substituted alkyl. In certain embodiments, at least one of R³ to R¹⁰ is alkoxy or substituted alkoxy. In certain embodiments, at least one of R³ to R¹⁰ is halogen. In certain embodiments, at least one of R³ to R¹⁰ is CF₃. In certain embodiments, at least one of R³ to R¹⁰ is carboxy. In certain embodiments, at least one of R³ to R¹⁰ is phosphonate. In certain embodiments, at least one of R³ to R¹⁰ is carboxylate. In certain embodiments, at least one of R³ to R¹⁰ is cyano. In certain embodiments, at least one of R³ to R¹⁰ is hydroxyl. In certain embodiments, at least one of R³ to R¹⁰ is carboxyamide. In certain embodiments, at least one of R³ to R¹⁰ is sulfonamide. In certain embodiments, at least one of R³ to R¹⁰ is sulfonate. In certain embodiments, at least one of R³ to R¹⁰ is amino or substituted amino. In certain embodiments, at least one of R³ to R¹⁰ is amide. In certain embodiments, at least one of R³ to R¹⁰ is aryl or substituted aryl. In certain embodiments, at least one of R³ to R¹⁰ is heteroaryl or substituted heteroaryl. In certain embodiments, at least one of R³ to R¹⁰ is heterocycle or substituted heterocycle.

In certain embodiments of formula (A1) or (A1a), at least one of R³ to R¹⁰ is a water-soluble group as described herein. In certain embodiments, at least two of R³ to R¹⁰ are water-soluble groups. In certain embodiments, the water-soluble group is a sulfonate.

In certain embodiments of formula (A1) or (A1a), R³ and R⁴ together with the carbon to which they are attached form an aryl or substituted aryl group. In certain embodiments of formula (A1), R³ and R⁴ together with the carbon to which they are attached form a heteroaryl or substituted heteroaryl. In certain embodiments of formula (A1) or (A1a), R³ and R⁴ together with the carbon to which they are attached form a heterocycle or substituted heterocycle. In certain embodiments of formula (A1) or (A1a), R³ and R⁴ together with the carbon to which they are attached form a cycloalkyl or substituted cycloalkyl. In some cases, the aryl, heteroaryl, heterocycle or cycloalkyl group is substituted with a water-soluble group, e.g., as described herein. In some cases, the water-soluble group is a sulfonate.

In certain embodiments of formula (A1) or (A1a), R⁷ and R⁸ together with the carbon to which they are attached form an aryl or substituted aryl group. In certain embodiments of formula (A1) or (A1a), R⁷ and R⁸ together with the carbon to which they are attached form a heteroaryl or substituted heteroaryl. In certain embodiments of formula (A1) or (A1a), R⁷ and R⁸ together with the carbon to which they are attached form a heterocycle or substituted heterocycle. In certain embodiments of formula (A1) or (A1a), R⁷ and R⁸ together with the carbon to which they are attached form a cycloalkyl or substituted cycloalkyl. In some cases, the aryl, heteroaryl, heterocycle or cycloalkyl group is substituted with a water-soluble group, e.g., as described herein. In some cases, the water-soluble group is a sulfonate.

In certain embodiments of formula (A1) or (A1a), X³ is a heteroatom or a substituted heteroatom. In certain cases, the heteroatom is O. In certain cases, the heteroatom is S. In certain other cases, X³ is NR²², wherein R²² is as described herein. In certain cases, R²² is H. In certain other cases, R²² is alkyl, such as methyl. In certain cases, X³ is CH₂ or a substituted carbon atom. In certain cases, X³ is a substituted carbon atom, wherein the substituent on the carbon atom is selected from alkyl, substituted alkyl, alkoxy, substituted alkoxy, halogen, CF₃, sulfonate, amino, substituted amino, amide, aryl, substituted aryl, heteroaryl, substituted heteroaryl, heterocycle and substituted heterocycle.

In certain embodiments of formula (A1) or (A1a), q is 1. In certain embodiments, q is more than 1, such as 2, 3, 4, 5, 6, 7, 8, 9, 10, 11, 12, 13, 14, 15, 16, 17, 18, 19 or 20.

In certain embodiments of formula (A1) or (A1a), R²² is H. In certain cases, R²² is alkyl or substituted alkyl. In certain cases, the alkyl group is a methyl group. In certain cases, R²² is aryl or substituted aryl. In certain cases, R²² is a heterocycle or substituted heterocycle. In certain cases, R²² is heteroaryl or substituted heteroaryl.

In certain embodiments of formula (A1) or (A1a), each of R¹⁶, R¹⁷, R¹⁸ and R¹⁹ are H. In certain cases, at least one of R¹⁶, R¹⁷, R¹⁸ and R¹⁹ is a substituent selected from alkyl, substituted alkyl, alkoxy, substituted alkoxy, halogen, CF³, sulfonate, amino, substituted amino, amide, aryl, substituted aryl, heteroaryl, substituted heteroaryl, heterocycle and substituted heterocycle.

In certain embodiments of formula (A1) or (A1a), at least one of R¹⁶, R¹⁷, R¹⁸ and R¹⁹ is alkyl or substituted alkyl. In certain embodiments, at least one of R¹⁶, R¹⁷, R¹⁸ and R¹⁹ is alkoxy or substituted alkoxy. In certain embodiments, at least one of R¹⁶, R¹⁷, R¹⁸ and R¹⁹ is halogen. In certain embodiments, at least one of R¹⁶, R¹⁷, R¹⁸ and R¹⁹ is CF₃. In certain embodiments, at least one of R¹⁶, R¹⁷, R¹⁸ and R¹⁹ is sulfonate. In certain embodiments, at least one of R¹⁶, R¹⁷, R¹⁸ and R¹⁹ is amino or substituted amino. In certain embodiments, at least one of R¹⁶, R¹⁷, R¹⁸ and R¹⁰ is amide. In certain embodiments, at least one of R¹⁶, R¹⁷, R¹⁸ and R¹⁹ is aryl or substituted aryl. In certain embodiments, at least one of R¹⁶, R¹⁷, R¹⁸ and R¹⁹ is heteroaryl or substituted heteroaryl. In certain embodiments, at least one of R¹⁶, R¹⁷, R¹⁸ and R¹⁹ is heterocycle or substituted heterocycle.

In certain embodiments of formula (A1), the functional dye is described by a structure selected from:

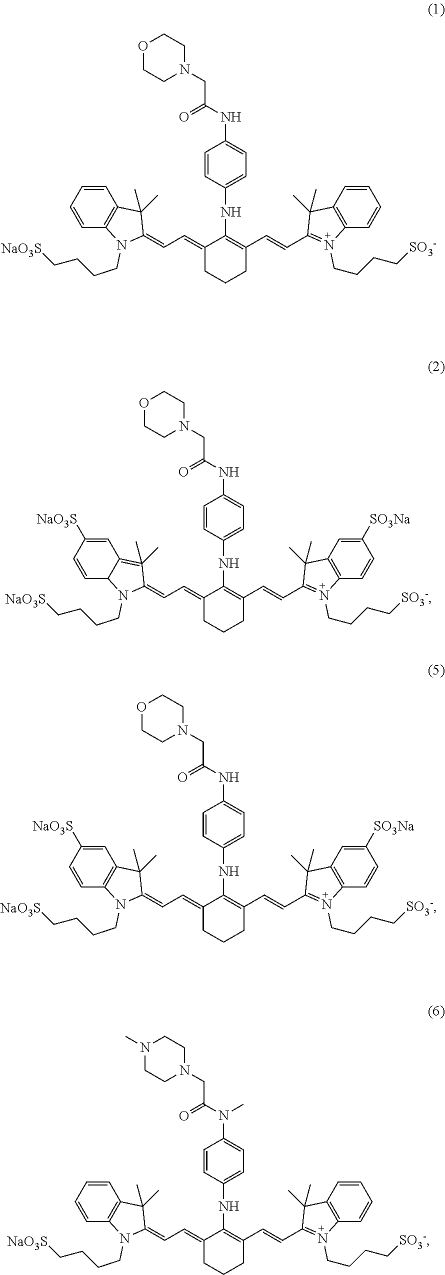

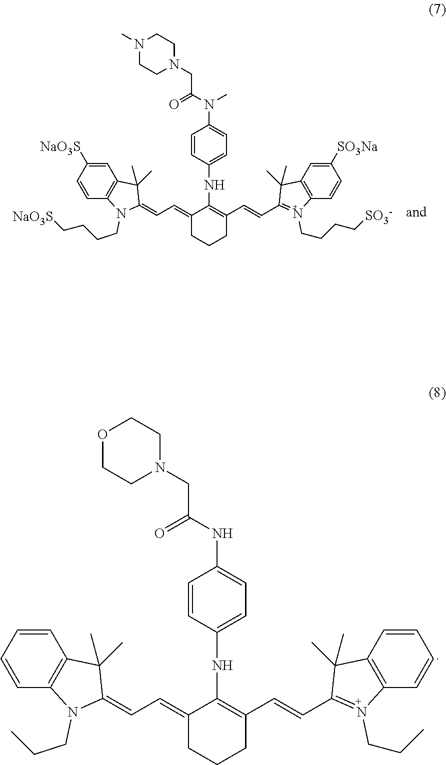

In certain embodiments of formula (A1a), the functional dye is described by a structure selected from:

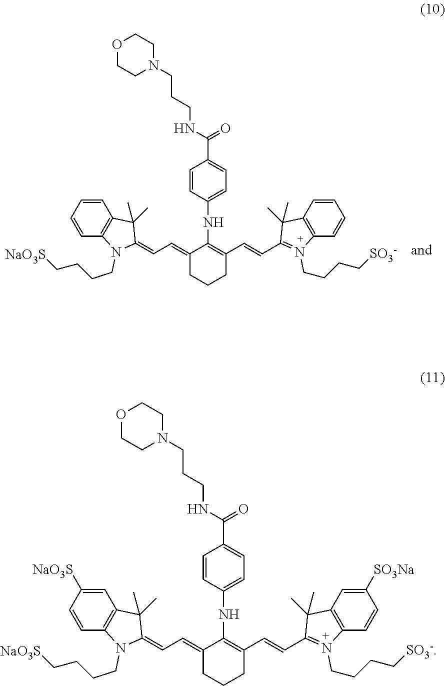

In certain embodiments, the subject functional dye is of the formula (A2):

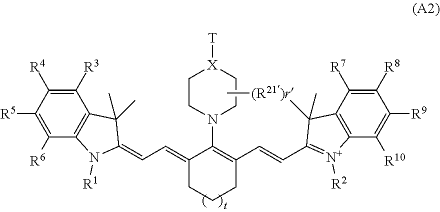

wherein:
R¹ and R² are each independently selected from H, alkyl, and (CH₂)ₙR¹², wherein n is an integer from 1 to 20 and R¹² is selected from the group consisting of H, sulfonate, carboxylic acid, amine, quaternary ammonium cation, phosphate, ester, halogen, azide, cyano, alkyne, and heterocycle;
R³ and R⁴, are each independently selected from H, alkyl, substituted alkyl, alkoxy, substituted alkoxy, halogen, CF₃, carboxy, phosphonate, carboxylate, cyano, hydroxyl, carboxyamide, sulfonamide, sulfonate, amino, substituted amino, amide, aryl, substituted aryl, heteroaryl, substituted heteroaryl, heterocycle and substituted heterocycle;
or R³ and R⁴ together with the carbon to which they are attached form a group selected from aryl, substituted aryl, heteroaryl, substituted heteroaryl, heterocycle, substituted heterocycle, cycloalkyl and substituted cycloalkyl;
R⁷ and R⁸ are each independently selected from H, alkyl, substituted alkyl, alkoxy, substituted alkoxy, halogen, CF₃, carboxy, phosphonate, carboxylate, cyano, hydroxyl, carboxyamide, sulfonamide, sulfonate, amino, substituted amino, amide, aryl, substituted aryl, heteroaryl, substituted heteroaryl, heterocycle and substituted heterocycle;
or R⁷ and R⁸ together with the carbon to which they are attached form a group selected from aryl, substituted aryl, heteroaryl, substituted heteroaryl, heterocycle, substituted heterocycle, cycloalkyl and substituted cycloalkyl; and
R⁵, R⁶, R⁹ and R¹⁰ are each independently selected from H, alkyl, substituted alkyl, alkoxy, substituted alkoxy, halogen, CF₃, carboxy, phosphonate, carboxylate, cyano, hydroxyl, carboxyamide, sulfonamide, sulfonate, amino, substituted amino, amide, aryl, substituted aryl, heteroaryl, substituted heteroaryl, heterocycle and substituted heterocycle;
t is an integer from 0 to 1;
X is selected from a N, CH and CR³², wherein R³² is selected from alkyl, substituted alkyl, hydroxy, alkoxy, substituted alkoxy, halogen, CF₃, sulfonate, amino, substituted amino, amide, aryl, substituted aryl, heteroaryl, substituted heteroaryl, heterocycle and substituted heterocycle;
T is a protein targeting moiety comprising a fragment of a known drug that targets a protein selected from the group consisting of (non-)receptor kinases, immune checkpoint proteins, G-protein-coupled receptors (GPCRs), influx transporters, efflux transporters, ion channels, human leukocyte antigens (HLA), proteases, caspases and nuclear receptors from a small molecule kinase inhibitor or a fragment of a small molecule modulator of GPCRs;
R^{21′} are each independently selected from alkyl, substituted alkyl, alkoxy, substituted alkoxy, halogen, CF₃, sulfonate, amino, substituted amino, amide, aryl, substituted aryl, heteroaryl, substituted heteroaryl, heterocycle and substituted heterocycle; and
r′ is an integer from 0 to 8.

In some embodiments of formula (A2), R¹ and R² are both (CH₂)ₙR¹² and each R¹² is independently selected from the group consisting of sulfonate, carboxylic acid, amine, quaternary ammonium cation, phosphate, ester, halogen, azide, cyano, alkyne, and heterocycle. In certain cases, each R¹² group are the same. In other cases, each R¹² group are different. In certain cases, at least one R¹² group is a sulfonate or a salt thereof. In some cases, both R¹² groups are a sulfonate or a salt thereof. In certain cases, n is an integer less than 20, such as 15 or less, 10 or less, 5 or less, or even less. In some instances, n is an integer less than 10, such as 9, 8, 7, 6, 5, 4, 3, 2 or 1. In certain instances, n is 4. In certain instances, n is 3.

In certain cases of formula (A2), R¹ and R² are both (CH₂)ₙR¹², wherein n is 4 and R¹² is sulfonate or a salt thereof.

In certain embodiments of formula (A2), each of R³ to R¹⁰ are H. In certain cases, at least one of R³ to R¹⁰ is a substituent selected from alkyl, substituted alkyl, alkoxy, substituted alkoxy, halogen, CF³, sulfonate, amino, substituted amino, amide, aryl, substituted aryl, heteroaryl, substituted heteroaryl, heterocycle and substituted heterocycle.

In certain embodiments of formula (A2), at least one of R³ to R¹⁰ is alkyl or substituted alkyl. In certain embodiments, at least one of R³ to R¹⁰ is alkoxy or substituted alkoxy. In certain embodiments, at least one of R³ to R¹⁰ is halogen. In certain embodiments, at least one of R³ to R¹⁰ is CF₃. In certain embodiments, at least one of R³ to R¹⁰ is carboxy. In certain embodiments, at least one of R³ to R¹⁰ is phosphonate. In certain embodiments, at least one of R³ to R¹⁰ is carboxylate. In certain embodiments, at least one of R³ to R¹⁰ is cyano. In certain embodiments, at least one of R³ to R¹⁰ is hydroxyl. In certain embodiments, at least one of R³ to R¹⁰ is carboxyamide. In certain embodiments, at least one of R³ to R¹⁰ is sulfonamide. In certain embodiments, at least one of R³ to R¹⁰ is sulfonate. In certain embodiments, at least one of R³ to R¹⁰ is amino or substituted amino. In certain embodiments, at least one of R³ to R¹⁰ is amide. In certain embodiments, at least one of R³ to R¹⁰ is aryl or substituted aryl. In certain embodiments, at least one of R³ to R¹⁰ is heteroaryl or substituted heteroaryl. In certain embodiments, at least one of R³ to R¹⁰ is heterocycle or substituted heterocycle.

In certain embodiments of formula (A2), at least one of R³ to R¹⁰ is a water-soluble group as described herein. In certain embodiments, at least two of R³ to R¹⁰ are water-soluble groups. In certain embodiments, the water-soluble group is a sulfonate.

In certain embodiments of formula (A2), R³ and R⁴ together with the carbon to which they are attached form an aryl or substituted aryl group. In certain embodiments of formula (A2), R³ and R⁴ together with the carbon to which they are attached form a heteroaryl or substituted heteroaryl. In certain embodiments of formula (A2), R³ and R⁴ together with the carbon to which they are attached form a heterocycle or substituted heterocycle. In certain embodiments of formula (A2), R³ and R⁴ together with the carbon to which they are attached form a cycloalkyl or substituted cycloalkyl. In some cases, the aryl, heteroaryl, heterocycle or cycloalkyl group is substituted with a water-soluble group, e.g., as described herein. In some cases, the water-soluble group is a sulfonate.

In certain embodiments of formula (A2), R⁷ and R⁸ together with the carbon to which they are attached form an aryl or substituted aryl group. In certain embodiments of formula (A2), R⁷ and R⁸ together with the carbon to which they are attached form a heteroaryl or substituted heteroaryl. In certain embodiments of formula (A2), R⁷ and R⁸ together with the carbon to which they are attached form a heterocycle or substituted heterocycle. In certain embodiments of formula (A2), R⁷ and R⁸ together with the carbon to which they are attached form a cycloalkyl or substituted cycloalkyl. In some cases, the aryl, heteroaryl, heterocycle or cycloalkyl group is substituted with a water-soluble group, e.g., as described herein. In some cases, the water-soluble group is a sulfonate.

In certain embodiments of formula (A2), X is N. In certain cases, X is CH₂ or a substituted carbon atom. In certain cases, X is CR³², wherein R³² is selected from alkyl, substituted alkyl, hydroxy, alkoxy, substituted alkoxy, halogen, CF₃, sulfonate, amino, substituted amino, amide, aryl, substituted aryl, heteroaryl, substituted heteroaryl, heterocycle and substituted heterocycle. In certain cases, R³² is as defined for group T. In certain cases, X is CR³² and R³² is hydroxy.

In certain embodiments of formula (A2), T is a fragment of a small molecule kinase inhibitor. In certain cases, the small molecule kinase inhibitor is selected from imatinib, bosutinib, nintedanib, ponatinib, brigatinib and dasatinib, ceritinib, erlotinib and palbociclib. In some cases, T is a fragment of imatinib, bosutinib, nintedanib, ponatinib, brigatinib or dasatinib.

In certain embodiments of formula (A2), T is a fragment of a small molecule modulator of G-protein-coupled receptors (GPCRs). In certain cases, the small molecule modulator of GPCRs is selected from sildenafil and eszopiclone.

In certain embodiments of formula (A2), T is a fragment of a small molecule androgen receptor inhibitor. In some cases, the androgen receptor inhibitor is enzalutamide.

In certain embodiments of formula (A2), T is a fragment of a small molecule immune checkpoint inhibitor such as a programmed cell death ligand 1 inhibitor (PD-L1 inhibitor). In some cases, the PD-L1 inhibitor is a compound of structure (9), or a fragment thereof.

In certain embodiments of formula (A2), r′ is 0 such that the compound of formula (A2) contains no R^{21′} substituents. In other cases, r′ is 1 such that the compound of formula (A2) contains one R^{21′} substituent. In other cases, r′ is 2 such that the compound of formula (A2) contains two R^{21′} substituents. In other cases, r′ is 3 such that the compound of formula (A2) contains three R^{21′} substituents. In other cases, r′ is 4 such that the compound of formula (A2) contains four R^{21′} substituents. In other cases, r′ is 5 such that the compound of formula (A2) contains five R^{21′} substituents. In other cases, r′ is 6 such that the compound of formula (A2) contains six R^{21′} substituents. In other cases, r′ is 7 such that the compound of formula (A2) contains seven R^{21′} substituents. In other cases, r′ is 8 such that the compound of formula (A2) contains eight R^{21′} substituents.

In certain embodiments of formula (A2), at least one R^{21′} substituent is alkyl or substituted alkyl. In certain embodiments, at least one R^{21′} substituent is alkoxy or substituted alkoxy. In certain embodiments, at least one R^{21′} substituent is halogen. In certain embodiments, at least one R^{21′} substituent is CF₃. In certain embodiments, at least one R^{21′} substituent is sulfonate. In certain embodiments, at least one R^{21′} substituent is amino or substituted amino. In certain embodiments, at least one R^{21′} substituent is amide. In certain embodiments, at least one R^{21′} substituent is aryl or substituted aryl. In certain embodiments, at least one R^{21′} substituent is heteroaryl or substituted heteroaryl. In certain embodiments, at least one R^{21′} substituent is heterocycle or substituted heterocycle.

In certain embodiments of a functional dye of formula (A2), T is selected from:

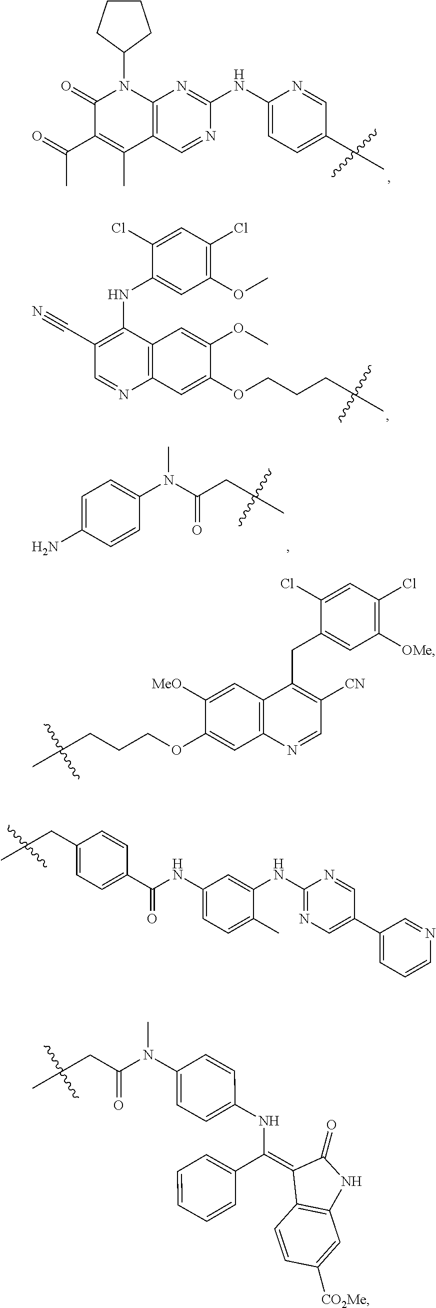

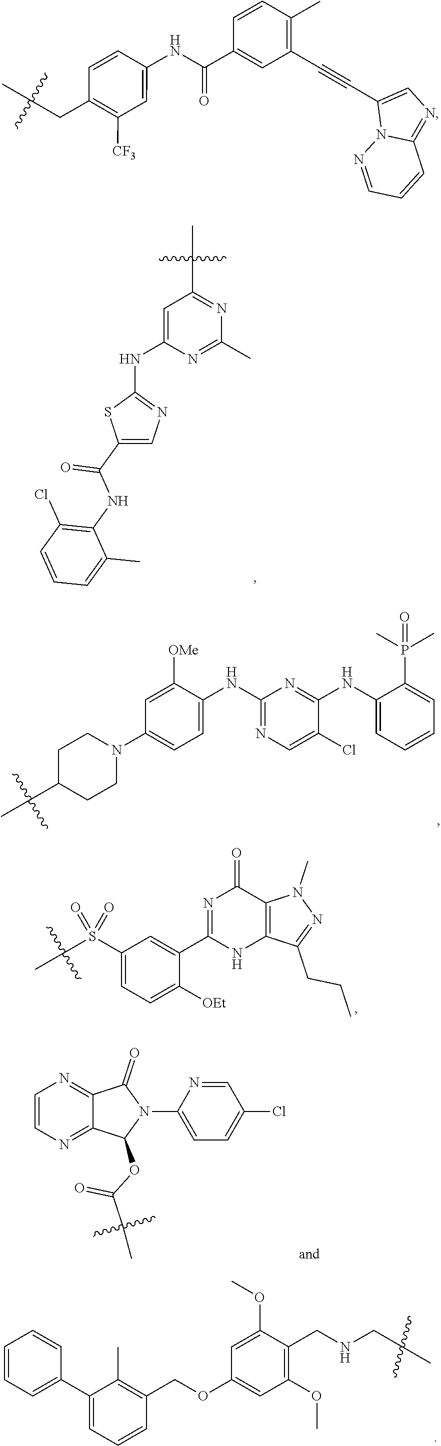

In certain embodiments, any one or more of the carbon atoms in a phenyl ring of any one of formulas (I)-(III) or (A1)-(A2) may be replaced by a heteroatom (e.g. O, S, N). For example, any one of the phenyl rings may be replaced by a heteroatom to form a pyridine, diazine, triazine, tetrazine, pyran, oxazine, thiopyran, thiazine, dioxine, dithiin, provided the structure is synthetically feasible.

In certain embodiments, the functional dye is described by the structure of compound 1 to 8 or by the formula (I), (II), (III) or (A1)-(A2). It is understood that any of the subject structures or formulas may be present in a salt form. In some cases, the salt form of the compound is a pharmaceutically acceptable salt.

Aspects of the present disclosure include functional dyes (e.g., as described herein), salts thereof (e.g., pharmaceutically acceptable salts), prodrug and/or solvate, hydrate forms thereof. In addition, it is understood that, in any compound described herein having one or more chiral centers, if an absolute stereochemistry is not expressly indicated, then each center may independently be of R-configuration or S-configuration or a mixture thereof. It will be appreciated that all permutations of salts, solvates, hydrates, prodrugs and stereoisomers are meant to be encompassed by the present disclosure.

In some embodiments, the subject functional dyes, or a prodrug form thereof, are provided in the form of pharmaceutically acceptable salts. Compounds containing an amine or nitrogen containing heteroaryl group may be basic in nature and accordingly may react with any number of inorganic and organic acids to form pharmaceutically acceptable acid addition salts. Acids commonly employed to form such salts include inorganic acids such as hydrochloric, hydrobromic, hydriodic, sulfuric and phosphoric acid, as well as organic acids such as para-toluenesulfonic, methanesulfonic, oxalic, para-bromophenylsulfonic, carbonic, succinic, citric, benzoic and acetic acid, and related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephathalate, sulfonate, xylenesulfonate, phenylacetate, phenylpropionate, phenylbutyrate, citrate, lactate, s-hydroxybutyrate, glycollate, maleate, tartrate, methanesulfonate, propanesulfonates, naphthalene-1-sulfonate, naphthalene-2-sulfonate, mandelate, hippurate, gluconate, lactobionate, and the like salts. In certain specific embodiments, pharmaceutically acceptable acid addition salts include those formed with mineral acids such as hydrochloric acid and hydrobromic acid, and those formed with organic acids such as fumaric acid and maleic acid.

In some embodiments, the subject compounds are provided in a prodrug form. “Prodrug” refers to a derivative of an active agent that requires a transformation within the body to release the active agent. In certain embodiments, the transformation is an enzymatic transformation. Prodrugs are frequently, although not necessarily, pharmacologically inactive until converted to the active agent. “Promoiety” refers to a form of protecting group that, when used to mask a functional group within an active agent, converts the active agent into a prodrug. In some cases, the promoiety will be attached to the drug via bond(s) that are cleaved by enzymatic or non-enzymatic means in vivo. Any convenient prodrug forms of the subject compounds can be prepared, e.g., according to the strategies and methods described by Rautio et al. (“Prodrugs: design and clinical applications”, Nature Reviews Drug Discovery 7, 255-270 (February 2008

In some embodiments, the subject compounds, prodrugs, stereoisomers or salts thereof are provided in the form of a solvate (e.g., a hydrate). The term “solvate” as used herein refers to a complex or aggregate formed by one or more molecules of a solute, e.g. a prodrug or a pharmaceutically-acceptable salt thereof, and one or more molecules of a solvent. Such solvates are typically crystalline solids having a substantially fixed molar ratio of solute and solvent. Representative solvents include byway of example, water, methanol, ethanol, isopropanol, acetic acid, and the like. When the solvent is water, the solvate formed is a hydrate.

The term “solvate” as used herein refers to a complex or aggregate formed by one or more molecules of a solute, e.g. a pharmaceutically-acceptable salt thereof, and one or more molecules of a solvent. Such solvates are typically crystalline solids having a substantially fixed molar ratio of solute and solvent. Representative solvents include by way of example, water, methanol, ethanol, isopropanol, acetic acid, and the like. When the solvent is water, the solvate formed is a hydrate.

In some embodiments, the subject functional dye is a non-systemic functional dye. As used herein, “non-systemic” refers to local administration, such that the subject functional dye does not come into contact with more than a limited portion of the host, such as less than 50%, less than 25%, less than 10%, or even less by volume of the host will be contacted with the subject functional dye that is delivered to the host by the subject methods. In other embodiments, the subject functional dye is a systemic functional dye. In some embodiments, the subject compounds are provided by oral dosing and absorbed into the bloodstream. In some embodiments, the oral bioavailability of the subject compounds is 30% or more, such as 40% or more, 50% or more, 60% or more, or even more. Modifications may be made to the subject compounds or their formulations using any convenient methods to increase or decrease absorption across the gut lumen or their bioavailability.

In some embodiments, the subject functional dye compounds are metabolically stable (e.g., remain substantially intact in vivo during the half-life of the compound). In certain embodiments, the compounds have a half-life (e.g., an in vivo half-life) of 5 minutes or more, such as 10 minutes or more, 12 minutes or more, 15 minutes or more, 20 minutes or more, 30 minutes or more, 60 minutes or more, 2 hours or more, 6 hours or more, 12 hours or more, 24 hours or more, or even more.

### Compositions

The herein-discussed functional dyes can be formulated using any convenient excipients, reagents and methods. Compositions are provided in formulation with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7th ed., Lippincott, Williams, & Wilkins; and Handbook of Pharmaceutical Excipients (2000) A. H. Kibbe et al., eds., 3rd ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, the subject compound is formulated in an aqueous buffer. Suitable aqueous buffers include, but are not limited to, acetate, succinate, citrate, and phosphate buffers varying in strengths from 5 mM to 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures. In some embodiments, the subject compound is formulated for sustained release.

In certain embodiments, the pharmaceutical composition includes sodium bicarbonate. In certain embodiments, the pharmaceutical composition includes polyethylene glycol (PEG). In some cases, the PEG included in the composition is PEG3350.

The subject compounds may be administered in a unit dosage form and may be prepared by any methods well known in the art. Such methods include combining the subject compound with a pharmaceutically acceptable carrier or diluent which constitutes one or more accessory ingredients. A pharmaceutically acceptable carrier is selected on the basis of the chosen route of administration and standard pharmaceutical practice. Each carrier must be “pharmaceutically acceptable” in the sense of being compatible with the other ingredients of the formulation and not injurious to the subject. This carrier can be a solid or liquid and the type is generally chosen based on the type of administration being used.

Examples of suitable solid carriers include lactose, sucrose, gelatin, agar and bulk powders. Examples of suitable liquid carriers include water, pharmaceutically acceptable fats and oils, alcohols or other organic solvents, including esters, emulsions, syrups or elixirs, suspensions, solutions and/or suspensions, and solution and or suspensions reconstituted from non-effervescent granules and effervescent preparations reconstituted from effervescent granules. Such liquid carriers may contain, for example, suitable solvents, preservatives, emulsifying agents, suspending agents, diluents, sweeteners, thickeners, and melting agents. Preferred carriers are edible oils, for example, corn or canola oils. Polyethylene glycols (PEG), e.g. PEG3350, are also good carriers.

For oral preparations, the functional dye can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Furthermore, a functional dye can be made into suppositories by mixing with a variety of bases such as emulsifying bases or water-soluble bases. A functional dye can be administered rectally via a suppository. The suppository can include vehicles such as cocoa butter, carbowaxes and polyethylene glycol monomethyl ethers, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the functional dye. Similarly, unit dosage forms for injection or intravenous administration may comprise a functional dye in a composition as a solution in sterile water, normal saline or another pharmaceutically acceptable carrier.

A functional dye can be formulated for administration by injection. Typically, injectable compositions are prepared as liquid solutions or suspensions; solid forms suitable for solution in, or suspension in, liquid vehicles prior to injection may also be prepared. The preparation may also be emulsified or the active ingredient encapsulated in liposome vehicles.

In certain embodiments, the functional dye can be formulated for administration by topical administration, e.g., via liquid, gel, ointment or fluid which contains the functional dye and can be applied directly to the area of interest. In some cases, the functional dye can be formulated for administration as a spray during endoscopy. Other formulations can be configured for administration of the functional dye via a surface adherent device.

### Methods

The functional dyes, and pharmaceutical compositions can be administered orally, sublingually, parentally, by spraying, by inhalation, topically, rectally, nasally, buccally, vaginally, or via an implanted reservoir. In certain cases, the composition is administered as a mouthwash. The term “parental administration” includes intravenous, intramuscular, subcutaneous, intraarterial, intraarticular, intrasynovial, intrasternal, intrathecal, intraperitoneal, intracisternal, intrahepatic, intralesional, intracranial and intralymphatic injection or infusion techniques. In some embodiments, the administration is intravenous. In some embodiments, the administration is intramuscular. In some embodiments, the administration is subcutaneous. In some embodiments, the administration is intravesicular. The functional dyes can also be administered via catheters or through a needle to a tissue. In some embodiments, the administration is percutaneous. In some embodiments, administration is oral. In some embodiments, the administration is sublingual. In some embodiments, the administration is topical. In some embodiments, administration is via a topical spray. In some embodiments, the administration is transdermal, e.g., via a patch.

In one embodiment, an effective amount (which is an amount effective to cause or increase fluorescence) of the functional dyes are administered. In one embodiment, between about 1 ng/kg and about 100 mg/kg, between about 100 ng/kg and 10 mg/kg, between about 1 μg/kg and about 5 mg/kg, between about 10 μg/kg and about 2 mg/kg, between about 50 μg/kg and about 1 mg/kg of the compound of the present invention is administered.

Since pre-malignant lesions have a high probability of progressing to colorectal cancer in the future, detection results obtained according to the detection method of the present invention serve as extremely useful information when assessing the risk of existing cancer, including colorectal cancer, and during minimally invasive assessment of the risk of future colorectal cancer at an early stage. For example, according to the detection method of the present invention, the subject can be assessed has having a high risk of the onset of colorectal cancer and colorectal adenoma in the future.

Conditions of interest for imaging include, without limitation, inflammatory conditions, a pathogen infection, precancer conditions, cancers and cancer related conditions. In some embodiments, the condition is an immune-related disease, disorder or condition. In some embodiments, the condition is a viral-related disease, disorder or condition.

In some instances of the subject methods, the target disease or condition is an inflammatory condition. Inflammatory conditions of interest include, but are not limited to, chronic inflammatory diseases (e.g., cardiovascular disease), cancer, multiple sclerosis (MS), experimental autoimmune encephalomyelitis (EAE), and oxidation stress related conditions.

In some instances of the subject methods, the target disease is a pathogen infection. In some embodiments of the methods of treatment, the infective disease condition results from infection with a positive-stranded RNA virus, negative stranded RNA virus, or a DNA virus. In some embodiments, the infective disease condition results from infection with a pathogen selected from the group of viral families consisting of Picomaviridae, Flaviviridae, Retroviridae, Filoviridae, Togaviridae, Papovaviridae, Caliciviridae, Coronavirinae, Hepeviridae, Bunyaviridae, Poxviridae and Orthomyxoviridae. In some embodiments, the infective disease condition results from infection with a pathogen selected from the phylum Apicomplexa or from the order Kinetoplastida. In some embodiments, the infective disease condition results from infection with a bacterium. In some embodiments, the infective disease condition results from infection with a fungus.

In some instances of the subject methods, the target disease or condition is a precancerous condition, e.g., a condition or lesion involving abnormal cells which are associated with an increased risk of developing into cancer. The precancerous condition or lesion may be in a variety of organ systems, including but not limited to, the skin, oral cavity, stomach, colon and hematological system. Examples of precancerous conditions include, without limitation, colon polyps, monoclonal gammopathy, cervical dysplasia, benign neoplasias, dysplasia.

In some instances of the subject methods, the target disease or condition is a cancer. Cancer conditions of interest which can be targeted according to the subject methods include a wide variety of cancer cells. In some instances, the cancer cell is selected from bladder, breast, colon, endometrial, cervix, testicle, liver, lung, non-small cell lung cancer (NSCLC), ovarian, prostate, pancreatic, brain, thyroid, stomach, kidney, melanoma and sarcoma cancer cells.

Conditions of interest for imaging include, without limitation, hyperproliferative conditions of the GI tract, including malignant and premalignant lesions. The term GI tract includes, for example, oral cavity, esophagus, stomach, small intestine, and large intestine. Gastrointestinal cancer refers to malignant conditions of the GI tract and accessory organs of digestion, including the esophagus, stomach, biliary system, pancreas, small intestine, large intestine, rectum and anus. The symptoms relate to the organ affected and can include obstruction (leading to difficulty swallowing or defecating), abnormal bleeding or other associated problems. The diagnosis often requires endoscopy, followed by biopsy of suspicious tissue. Conditions of interest for imaging also include, without limitation, hyperproliferative conditions of the breast, brain, prostate, pancreas, skin, bladder, head, neck and thyroid, including malignant and premalignant lesions. In certain cases, the lesion is a cancerous lesion. In certain cases, the lesion is a precancerous lesion.

Esophageal cancer is the sixth-most-common cancer in the world. There are two main types of esophageal cancer-adenocarcinoma and squamous cell carcinoma. Adenocarcinomas of the esophagus tend to arise in a field defect called Barrett's esophagus, a red patch of tissue in the generally pink lower esophagus. Esophageal squamous-cell carcinomas may occur as second primary tumors associated with head and neck cancer. Cancer of the stomach, also called gastric cancer, is the fourth-most-common type of cancer. The most common type of gastric cancer is adenocarcinoma. Pancreatic cancer is the fifth-most-common cause of cancer deaths in the United States. These cancers are classified as endocrine or nonendocrine tumors. The most common is ductal adenocarcinoma. Colorectal cancer may be associated with hereditary syndromes like Peutz-Jegher's, hereditary nonpolyposis colorectal cancer or familial adenomatous polyposis, or may be age related. Colorectal cancer can be detected through the bleeding of a polyp, colicky bowel pain, a bowel obstruction or the biopsy of a polyp at a screening colonoscopy. Anal cancers include carcinomas and squamous cell carcinomas.

The general principles of fluorescence, optical image acquisition, and image processing can be applied in the practice of the invention. For a review of optical imaging techniques, see, e.g., Alfano et al., Ann. NY Acad. Sci. 820:248-270, 1997. An imaging system useful in the practice of methods described herein typically includes three basic components: (1) an appropriate light source for fluorescent molecule excitation, (2) a means for separating or distinguishing emissions from light used for the excitation, and (3) a detection system to detect the optical signal emitted.

In general, the optical detection system can be viewed as including an optical gathering/image forming component and an optical detection/image recording component. The optical detection system can be a single integrated device that incorporates both components.

A particularly useful optical gathering/image forming component is an endoscope. Endoscopic devices and techniques which have been used for in vivo optical imaging of numerous tissues and organs, including peritoneum, colon and rectum, bile ducts, stomach, bladder, lung, brain, esophagus, and head and neck regions can be employed in the practice of the present invention. Other types of optical gathering components useful in the invention are catheter-based devices, including fiber optics devices. Still other imaging technologies, including phased array technology, optical tomography, intravital microscopy, confocal imaging and fluorescence molecular tomography (FMT) can be employed in the practice of the present invention. In certain cases, the functional dye is detected with a fluorescent endoscopic camera or a fluorescent bronchoscopic camera. In certain cases, the functional dye is detected using a fluorescence capsule endoscopic camera, e.g., by Video Capsule Endoscopy. For a review see, for example Pasha (2018) Curr Gastroenterol Rep. 20(5):22.

A suitable optical detection/image recording component, e.g., charge coupled device (CCD) systems or photographic film, can be used in the invention. The choice of optical detection/image recording will depend on factors including type of optical gathering/image forming component being used. Selecting suitable components, assembling them into an optical imaging system, and operating the system is within ordinary skill in the art.

In embodiments where the functional dye is administered topically, e.g., as a mouthwash, a suitable smart phone app may be used for detection of the functional dye. For a review, see Hernández-Neuta et al. (2019) JIM 285:19-39.

### Diagnostic and Disease Applications and Methods

The methods described herein can be used to determine a number of indicia, including tracking the localization of the functional dyes in the subject over time, or assessing changes in the subject over time. The methods can also be used to follow therapy for such diseases by imaging molecular events and biological pathways.

The methods can be used to help a physician or surgeon to identify and characterize areas of disease, such as pre-malignant lesions, cancers and specifically colon polyps, to distinguish diseased and normal tissue, help dictate a therapeutic or surgical intervention, e.g., by determining whether a lesion is cancerous and should be removed or non-cancerous and left alone, or in surgically staging a disease. In certain cases, the methods can be used to help a radiologist or gastroenterologist. In certain cases of the subject methods, detection of the functional dye is used as a fluorescence-guided diagnosis tool. In certain cases of the subject methods, detection of the functional dye is used as a biopsy guide, e.g. biopsy needle, or optical mammography. In certain cases of the subject methods, detection of the functional dye is used in optical-guided intervention. In certain cases of the subject methods, detection of the functional dye is used as fluorescence-guided surgery. In certain cases the fluorescence-guided surgery is robotically assisted surgery. In certain cases of the subject methods, detection of the functional dye is used to guide surgery of a lesion.

In one embodiment, there is provided a method of performing image-guided surgery on a patient having a precancerous or cancerous lesion, the method comprising: a) administering a subject functional dye (e.g., including one or more compounds as described herein): b) detecting the functional dye, wherein detection of the functional dye is indicative of a lesion; and b) using at least one image of the lesion and surrounding tissue to determine where resection of the lesion is needed. Imaging of the lesion may be performed, for example, pre-operatively to assist surgical planning and/or intra-operatively to provide image-guidance during surgery, e.g., for lesion margin delineation or evaluation of completeness of resection.

In certain embodiments of the subject methods, detection of the functional dye/lesion is with a fluorescence endoscopic camera or a fluorescence bronchoscopic camera. In certain cases, the functional dye/lesion is detected with a fluorescence capsule endoscopic camera, a cystoscope or a laparoscope. In certain cases, the camera used for detection is a combined white light near-infrared (WL/NIRF) camera.

In certain other embodiments of the subject methods, detection of the functional dye is used for treatment monitoring. In some cases, the subject methods are used in an ambulant setting, e.g., imaging in an outpatient. For example, the subject functional dye may be administered to a patient, e.g., orally or topically, and the patient may be monitored over a period of time. For example, the patient is equipped with a portable or wearable device which can count the fluorescent circulating cells over a period of time, e.g., tumor cells, at home or in a remote location. In some cases, the portable or wearable device is a wearable cytometer. In some cases, the wearable cytometer non-invasively, counts circulating cells (e.g. cancer cells, white blood cells, etc.) labeled with the functional dye optically from the patient (e.g., through the skin of the patient's wrist), performs analysis by cytometry, then sends the data to an electronic device (e.g., a smartphone app).

The methods of the invention can also be used in the detection, characterization and/or determination of the localization of a disease, especially early disease, the severity of a disease or a disease-associated condition, the staging of a disease, and monitoring and guiding various therapeutic interventions, such as surgical procedures, and monitoring drug therapy, including cell-based therapies.

The methods of the invention can therefore be used, for example, to determine the presence of tumor cells and localization of tumor cells. The methods and compounds (compositions) of the invention can also be used in identification and evaluation of apoptosis, necrosis, hypoxia and angiogenesis. The subject methods can be used to determine the presence of a lesion in one or more locations selected from the gastrointestinal tract, breast, brain, prostate, pancreas, skin, bladder, head, neck and thyroid, cervix.

The compounds (compositions) and methods of the present invention can be used in combination with other imaging compositions and methods. For example, the methods of the present invention can be used in combination with other traditional imaging modalities such as X-ray, computed tomography (CT), positron emission tomography (PET), single photon computerized tomography (SPECT), and magnetic resonance imaging (MRI). For instance, the compounds (compositions) and methods of the present invention can be used in combination with CT and MR imaging to obtain both anatomical and biological information simultaneously, for example, by co-registration of a tomographic image with an image generated by another imaging modality. In particular, the combination with MRI or CT is preferable, given the high spatial resolution of these imaging techniques. The compounds (compositions) and methods of the present invention can also be used in combination with X-ray, CT, PET, SPECT and MR contrast agents or the fluorescent silicon nanoparticle imaging probes of the present invention may also contain components, such as iodine, gadolinium atoms and radioactive isotopes, which can be detected using CT, PET, SPECT, and MR imaging modalities in combination with optical imaging.

Accordingly, in certain cases, the functional dye is multimodal. In certain embodiments, the imaging of the functional dye is performed using one or more additional methods selected from ultrasound imaging (UI), positron emission tomography (PET), single photon emission computed tomography (SPECT), magnetic resonance imaging (MRI), computed tomography (CT), optical imaging (01), photoacoustic imaging (PI).

In some embodiments, the functional dye can include various labels, including but not limited to, an isotopic label, a chemiluminescent label, a bioluminescent label, a paramagnetic ion, an enzyme, or a photoactive agent.

Exemplary isotopic labels may comprise radioactive isotopes (e.g., gamma-emitters, beta-emitters, and positron-emitters) or non-radioactive isotopes (e.g., stable trace isotopes), such as, but not limited to:

²H, ¹²⁰I, ¹²³I, ¹²⁴I, ¹²⁵I, ¹³¹I, ²⁰⁹As, ²¹⁰As, ²¹¹As, ³⁵S, ^{U}C, ¹³C, ¹⁴C, ³²P, ¹⁵N, ¹³N, ¹¹⁰In, ¹¹¹In, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹⁵⁴Gd, ¹⁵⁵Gd, ¹⁵⁵Gd, ¹⁵⁷Gd, ¹⁵⁸Gd, ¹⁵O, ¹⁸⁶Re, ¹⁸⁸Re, ⁵¹M, ^{52m}Mn, ⁵⁵Co, ⁷²As, ⁷⁵Br, ⁷⁶Br, ^{82m}Rb and ⁸³Sr.

Exemplary paramagnetic ions include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III).

Exemplary agents for CT imaging include without limitation, gold, iodine and barium.

### Kits

The functional dyes described herein can be packaged as a kit, which may optionally include instructions for using the dyes in various exemplary applications. Non-limiting examples include kits that contain, e.g., the compounds (compositions) in a powder or lyophilized form, and instructions for using, including reconstituting, dosage information, and storage information for in vivo and/or in vitro applications. Kits may optionally contain containers of the dyes in a liquid form ready for use, or requiring further mixing with solutions for administration. For in vivo applications, the kit may contain the compounds (compositions) in a dosage and form suitable for a particular application, e.g. a liquid in a vial, a pill for ingestion, etc. In some embodiments, the kit includes the functional dye formulated as topical application, including but not limited to a mouth wash.

The kit can include optional components that aid in the administration of the unit dose to subjects, such as vials for reconstituting powder forms, etc. The kits may be supplied in either a container which is provided with a seal which is suitable for single or multiple puncturing with a hypodermic needle (e.g. a crimped-on septum seal closure) while maintaining sterile integrity. Such containers may contain single or multiple subject doses. Additionally, the unit dose kit can contain customized components that aid in the detection of the dyes in vivo or in vitro, e.g., specialized endoscopes (such as those used in capsule endoscopy), light filters. The kits may also contain instructions for preparation and administration of the compounds (compositions). The kit may be manufactured as a single use unit dose for one subject, multiple uses for a particular subject, or the kit may contain multiple doses suitable for administration to multiple subjects (“bulk packaging”). The kit components may be assembled in cartons, blister packs, bottles, tubes, and the like.

In some embodiments, the subject kits comprise, a subject functional dye (e.g., a compound or composition as described herein), and a capsule endoscope. In some cases, the subject kits comprise a subject functional dye and a wearable or portable device configured for detection of the functional dye.

In addition to the above components, the subject kits may further include instructions for practicing the subject methods. These instructions may be present in the subject kits in a variety of forms, one or more of which may be present in the kit One form in which these instructions may be present is as printed information on a suitable medium or substrate, e.g., a piece or pieces of paper on which the information is printed, in the packaging of the kit, in a package insert, etc. Another means would be a computer readable medium, e.g., CD, DVD, Blu-Ray, computer-readable memory (e.g., flash memory), etc., on which the information has been recorded or stored. Yet another means that may be present is a website address, e.g., such as a link to a website for downloading a suitable smart phone app for use in detecting the functional dye, which may be used via the Internet to access the information at a removed site. Any convenient means may be present in the kits.

### Additional Embodiments

Additional embodiments are set forth in the following clauses:

### Additional Embodiments

Clause 1. A functional dye of formula:

D-L-F
wherein:
D is a near infrared fluorescent dye;
L is an optional linker; and
F is a small molecule that targets a protein.

Clause 2. The functional dye of clause 1, wherein the small molecule is a known drug, or a fragment of a known drug that targets a protein selected from the group consisting of (non)-receptor kinases, immune checkpoint proteins, G-protein-coupled receptors (GPCRs), influx transporters, efflux transporters, ion channels, human leukocyte antigens (HLA), proteases, caspases and nuclear receptors.

Clause 3. The functional dye of clause 1 or 2, wherein the protein is selected from a wild type, a splice variant,

Clause 4. The functional dye of any one of clauses 1 to 3, wherein F comprises a small molecule kinase inhibitor or a small molecule kinase inhibitor fragment.

Clause 5. The functional dye of clause 4, wherein the small molecule kinase inhibitor is selected from ceritinib and palbociclib.

Clause 6. The functional dye of clause 4, wherein the small molecule kinase inhibitor fragment is selected from N-desmethyl imatinib, N-desmethyl bosutinib, N-desmethyl nintedanib, N-desmethyl ponatinib, N-desmethyl brigatinib, N-deshydroxyethyl dasatinib.

Clause 7. The functional dye of clause 4, wherein the small molecule kinase inhibitor fragment comprises a fragment of nintedanib or lapatinib.

Clause 8. The functional dye of any one of clauses 1 to 3, wherein F comprises a small molecule modulator of G-protein-coupled receptors (GPCRs) or a fragment of a small molecule modulator of G-protein-coupled receptors (GPCRs).

Clause 9. The functional dye of clause 8, wherein the small molecule modulator of G-protein-coupled receptors (GPCRs) is selected from N-desmethyl sildenafil and N-desmethyl eszopiclone.

Clause 10. The functional dye of clause 1, wherein F comprises a small molecule agonist of ion-channels.

Clause 11. The functional dye of clause 10, wherein the small molecule agonist of ion-channels is procainamide.

Clause 12. The functional dye of clause 1, wherein F comprises a small molecule of the following structure:

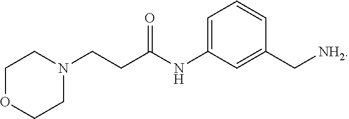 [SMILES: NCc1cccc(NC(=O)CCN2CCOCC2)c1]

Clause 13. The functional dye of clause 1, wherein F is selected from a fragment of formula (F1) and a fragment of formula (F2), wherein fragment (F1) is of the formula:

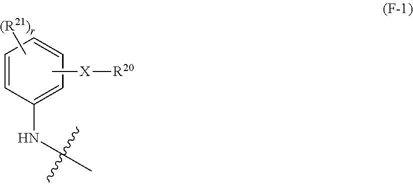

wherein:
X is selected from C(O)NR²², NR²², O and S, wherein R²² is selected from H, alkyl, substituted alkyl, aryl, substituted aryl, heterocycle, substituted heterocycle, heteroaryl and substituted heteroaryl;
R²⁰ is selected from substituted alkyl, substituted acyl, benzyl, substituted benzyl, aryl, substituted aryl, heteroaryl, substituted heteroaryl, heterocycle, substituted heterocycle;
R²¹ are each independently selected from alkyl, substituted alkyl, alkoxy, substituted alkoxy, halogen, CF₃, sulfonate, amino, substituted amino, amide, aryl, substituted aryl, heteroaryl, substituted heteroaryl, heterocycle and substituted heterocycle; and
r is an integer from 0 to 4; and fragment (F2) is of the formula

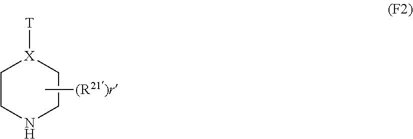

wherein:
X is selected from a N, CH and CR³², wherein R³² is selected from alkyl, substituted alkyl, hydroxy, alkoxy, substituted alkoxy, halogen, CF₃, sulfonate, amino, substituted amino, amide, aryl, substituted aryl, heteroaryl, substituted heteroaryl, heterocycle and substituted heterocycle;
T is a protein-targeting moiety comprising a fragment of a known drug that targets a protein selected from the group consisting of (non)-receptor kinases, immune checkpoint proteins, G-protein-coupled receptors (GPCRs), influx transporters, efflux transporters, ion channels, human leukocyte antigens (HLA), proteases, caspases and nuclear receptors from a small molecule kinase inhibitor or a fragment of a small molecule modulator of GPCRs;
R^{21′} are each independently selected from alkyl, substituted alkyl, alkoxy, substituted alkoxy, halogen, CF₃, sulfonate, amino, substituted amino, amide, aryl, substituted aryl, heteroaryl, substituted heteroaryl, heterocycle and substituted heterocycle; and
r′ is an integer from 0 to 8.

Clause 14. The functional dye of claim 13, wherein fragment (F1) is of the formula (F1A):

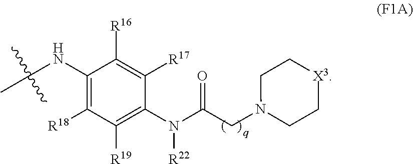

wherein:
q is an integer from 1 to 20;
R²² is selected from H, alkyl, substituted alkyl, aryl, substituted aryl, heterocycle, substituted heterocycle, heteroaryl, substituted heteroaryl;
R¹⁶, R¹⁷, R¹⁸ and R¹⁹ are each independently selected from H, alkyl, substituted alkyl, alkoxy, substituted alkoxy, halogen, CF³, sulfonate, amino, substituted amino, amide, aryl, substituted aryl, heteroaryl, substituted heteroaryl, heterocycle and substituted heterocycle; and
X³ is selected from a heteroatom, a substituted heteroatom, CH₂ and a substituted carbon atom.

Clause 15. The functional dye of clause 14, wherein formula (F1A) is of the structure:

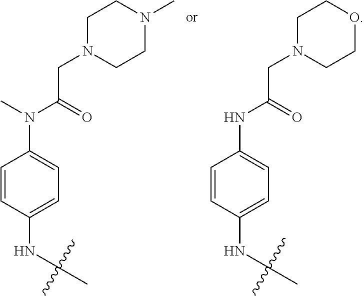

Clause 16. The functional dye of clause 13, wherein fragment (F1) is of the formula (F1B):

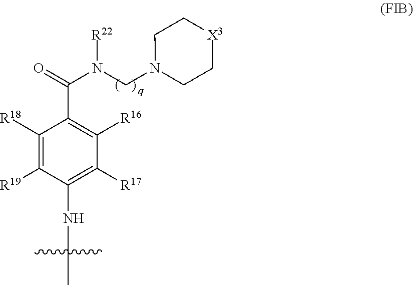

wherein:
q is an integer from 1 to 20;
R²² is selected from H, alkyl, substituted alkyl, aryl, substituted aryl, heterocycle, substituted heterocycle, heteroaryl, substituted heteroaryl;
R¹⁶, R¹⁷, R¹⁸ and R¹⁹ are each independently selected from H, alkyl, substituted alkyl, alkoxy, substituted alkoxy, halogen, CF³, sulfonate, amino, substituted amino, amide, aryl, substituted aryl, heteroaryl, substituted heteroaryl, heterocycle and substituted heterocycle; and
X³ is selected from a heteroatom, a substituted heteroatom, CH₂ and a substituted carbon atom.

Clause 17. The functional dye of clause 16, wherein formula (F1B) is of the structure:

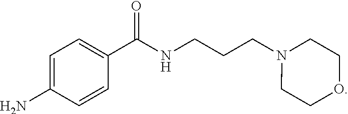 [SMILES: Nc1ccc(C(=O)NCCCN2CCOCC2)cc1]

Clause 18. The functional dye of claim 13, wherein fragment (F1) is of the formula (F1C):

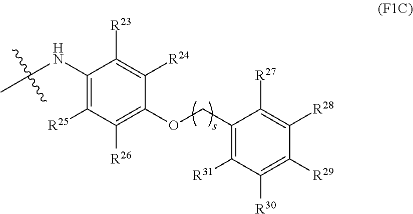

wherein:
s is an integer from 1 to 20; and
R²³ to R³¹ are each independently selected from H, alkyl, substituted alkyl, alkoxy, substituted alkoxy, halogen, CF₃, sulfonate, amino, substituted amino, amide, aryl, substituted aryl, heterocycle, substituted heterocycle, heterocycle and substituted heterocycle.

Clause 19. The functional dye of claim 18, wherein the formula (F1C) is of the structure:

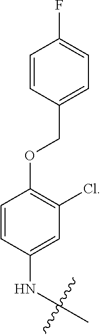 [SMILES: *Nc1ccc(OCc2ccc(F)cc2)c(Cl)c1]

Clause 20. The functional dye of claim 13, wherein the formula (F1) is of the structure:

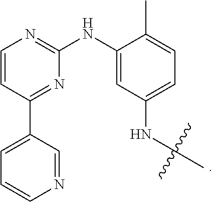 [SMILES: *Nc1ccc(C)c(Nc2nccc(-c3cccnc3)n2)c1]

Clause 21. The functional dye of any one of clauses 1 to 20, wherein the near infrared fluorescent dye is a cyanine (Cy) dye.

Clause 22. The functional dye of clause 21, wherein the cyanine dye is of the formula (A):

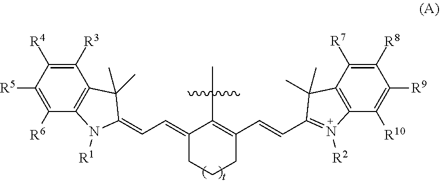

wherein:
R¹ and R² are each independently selected from H, alkyl, and (CH₂)ₙR¹², wherein n is an integer from 1 to 20 and R¹² is selected from the group consisting of H, sulfonate, carboxylic acid, amine, quaternary ammonium cation, phosphate, ester, halogen, azide, cyano, alkyne, and heterocycle;
R³ and R⁴, are each independently selected from H, alkyl, substituted alkyl, alkoxy, substituted alkoxy, halogen, CF₃, carboxy, phosphonate, carboxylate, cyano, hydroxyl, carboxyamide, sulfonamide, sulfonate, amino, substituted amino, amide, aryl, substituted aryl, heteroaryl, substituted heteroaryl, heterocycle and substituted heterocycle;
or R³ and R⁴ together with the carbon to which they are attached form a group selected from aryl, substituted aryl, heteroaryl, substituted heteroaryl, heterocycle, substituted heterocycle, cycloalkyl and substituted cycloalkyl;
R⁷ and R⁸ are each independently selected from H, alkyl, substituted alkyl, alkoxy, substituted alkoxy, halogen, CF₃, carboxy, phosphonate, carboxylate, cyano, hydroxyl, carboxyamide, sulfonamide, sulfonate, amino, substituted amino, amide, aryl, substituted aryl, heteroaryl, substituted heteroaryl, heterocycle and substituted heterocycle;
or R⁷ and R⁸ together with the carbon to which they are attached form a group selected from aryl, substituted aryl, heteroaryl, substituted heteroaryl, heterocycle, substituted heterocycle, cycloalkyl and substituted cycloalkyl;
R⁵, R⁶, R⁹ and R¹⁰ are each independently selected from H, alkyl, substituted alkyl, alkoxy, substituted alkoxy, halogen, CF₃, carboxy, phosphonate, carboxylate, cyano, hydroxyl, carboxyamide, sulfonamide, sulfonate, amino, substituted amino, amide, aryl, substituted aryl, heteroaryl, substituted heteroaryl, heterocycle and substituted heterocycle; and
t is an integer from 0 to 1.

Clause 23. The functional dye of clause 22, wherein the cyanine dye is substituted with at least one iodide.

Clause 24. The functional dye of clause 22 or 23, wherein the cyanine dye is substituted with at least one isotopic label.

Clause 25. The functional dye of clause 24, wherein the isotopic label is selected from ²H, ¹²⁰I, ¹²³I, ¹²⁴I, ¹²⁵I, ¹³¹I, ²⁰⁹As, ²¹⁰As, ²¹¹As, ³⁵S, ^{U}C, ¹³C, ¹⁴C, ³²P, ¹⁵N, ¹³N, ¹¹⁰In, ¹¹¹In, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹⁵⁴Gd, ¹⁵⁵Gd, ¹⁵⁵Gd, ¹⁵⁷Gd, ¹⁵⁸Gd, ¹⁵O, ¹⁸⁶Re, ¹⁸⁸Re, ⁵¹M, ^{52m}Mn, ⁵⁵Co, ⁷²As, ⁷⁵Br, ⁷⁶Br, ^{82m}Rb and ⁸³Sr.

Clause 26. The functional dye of clause 22, wherein the cyanine dye is selected from:

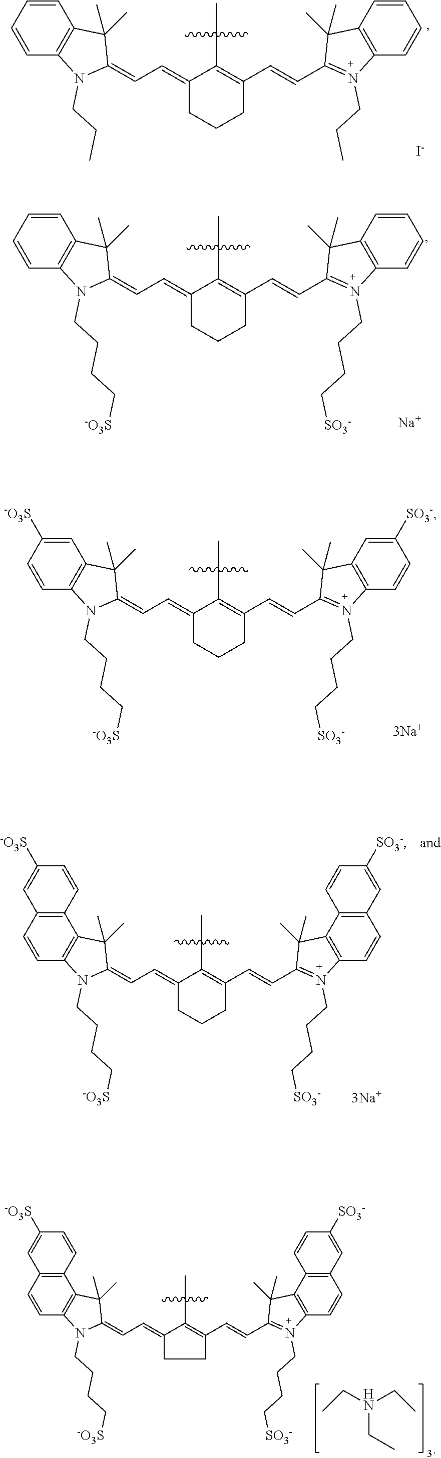

Clause 27. The functional dye of any one of clauses 1 to 20, wherein the near infrared fluorescent dye is fluorescein or a derivative thereof.

Clause 28. The functional dye of claim 27, wherein the fluorescein derivative is fluorescein isothiocyanate (FITC).

Clause 29. The functional dye of any one of clauses 1 to 20, wherein the near infrared fluorescent dye is rhodamine isothiocyanate (RITC).

Clause 30. The functional dye of any one of clauses 1 to 3, wherein the functional dye is of the formula (A1):

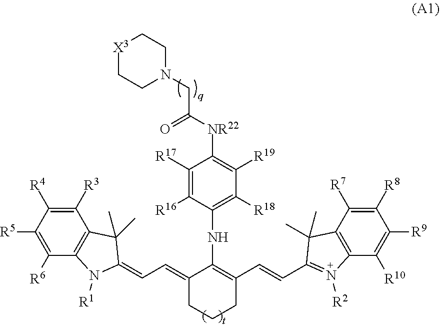

wherein:
R¹ and R² are each independently selected from H, alkyl, and (CH₂)ₙR¹², wherein n is an integer from 1 to 20 and R¹² is selected from the group consisting of H, sulfonate, carboxylic acid, amine, quaternary ammonium cation, phosphate, ester, halogen, azide, cyano, alkyne, and heterocycle;
R³ and R⁴, are each independently selected from H, alkyl, substituted alkyl, alkoxy, substituted alkoxy, halogen, CF₃, carboxy, phosphonate, carboxylate, cyano, hydroxyl, carboxyamide, sulfonamide, sulfonate, amino, substituted amino, amide, aryl, substituted aryl, heteroaryl, substituted heteroaryl, heterocycle and substituted heterocycle;
or R³ and R⁴ together with the carbon to which they are attached form a group selected from aryl, substituted aryl, heteroaryl, substituted heteroaryl, heterocycle, substituted heterocycle, cycloalkyl and substituted cycloalkyl;
R⁷ and R⁸ are each independently selected from H, alkyl, substituted alkyl, alkoxy, substituted alkoxy, halogen, CF₃, carboxy, phosphonate, carboxylate, cyano, hydroxyl, carboxyamide, sulfonamide, sulfonate, amino, substituted amino, amide, aryl, substituted aryl, heteroaryl, substituted heteroaryl, heterocycle and substituted heterocycle;
or R⁷ and R⁸ together with the carbon to which they are attached form a group selected from aryl, substituted aryl, heteroaryl, substituted heteroaryl, heterocycle, substituted heterocycle, cycloalkyl and substituted cycloalkyl; and
R⁵, R⁶, R⁹ and R¹⁰ are each independently selected from H, alkyl, substituted alkyl, alkoxy, substituted alkoxy, halogen, CF₃, carboxy, phosphonate, carboxylate, cyano, hydroxyl, carboxyamide, sulfonamide, sulfonate, amino, substituted amino, amide, aryl, substituted aryl, heteroaryl, substituted heteroaryl, heterocycle and substituted heterocycle;
T is an integer from 0 to 1;
q is an integer from 1 to 20;
R²² is selected from H, alkyl, substituted alkyl, aryl, substituted aryl, heterocycle, substituted heterocycle, heteroaryl, substituted heteroaryl;
R¹⁶, R¹⁷, R¹⁸ and R¹⁹ are each independently selected from H, alkyl, substituted alkyl, alkoxy, substituted alkoxy, halogen, CF₃, sulfonate, amino, substituted amino, amide, aryl, substituted aryl, heteroaryl, substituted heteroaryl, heterocycle and substituted heterocycle; and
X³ is selected from a heteroatom, a substituted heteroatom, CH₂ and a substituted carbon atom.

Clause 31. The functional dye of clause 30, selected from the following structures:

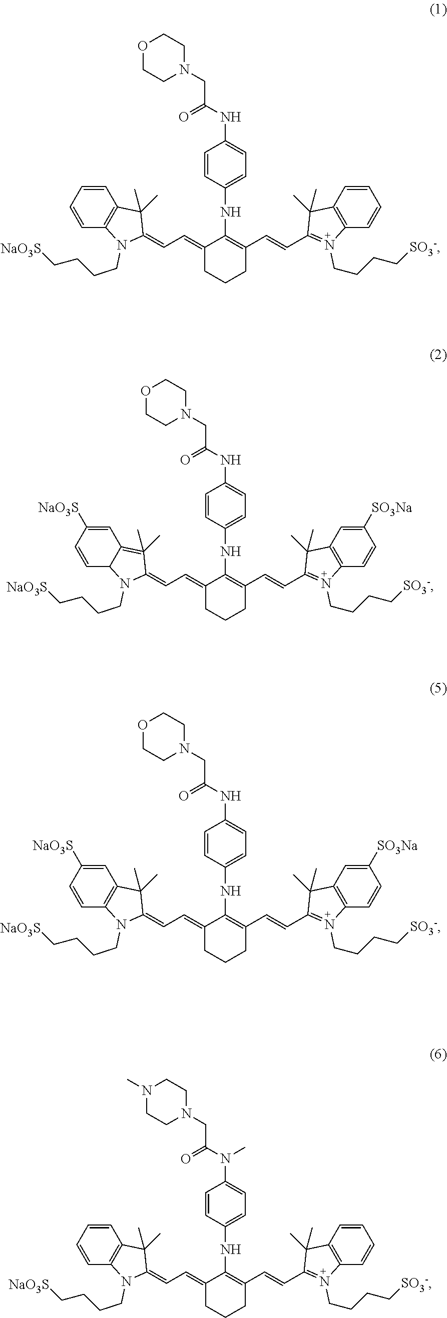

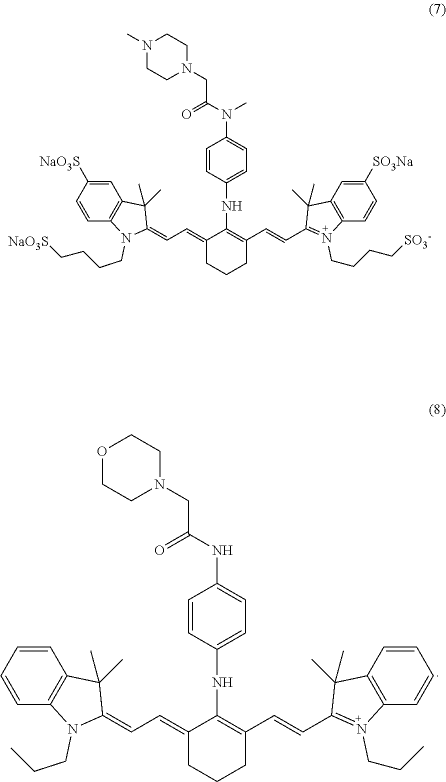

Clause 32. The functional dye of any one of clauses 1 to 3, wherein the functional dye is of the formula (A1a):

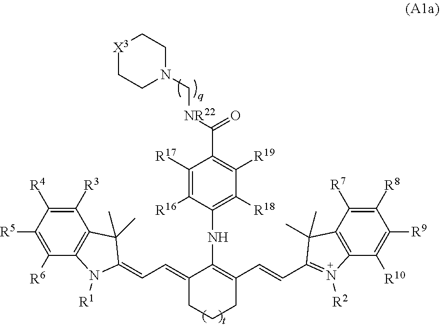

wherein:
R¹ and R² are each independently selected from H, alkyl, and (CH₂)ₙR¹², wherein n is an integer from 1 to 20 and R¹² is selected from the group consisting of H, sulfonate, carboxylic acid, amine, quaternary ammonium cation, phosphate, ester, halogen, azide, cyano, alkyne, and heterocycle;
R³ and R⁴, are each independently selected from H, alkyl, substituted alkyl, alkoxy, substituted alkoxy, halogen, CF₃, carboxy, phosphonate, carboxylate, cyano, hydroxyl, carboxyamide, sulfonamide, sulfonate, amino, substituted amino, amide, aryl, substituted aryl, heteroaryl, substituted heteroaryl, heterocycle and substituted heterocycle;
or R³ and R⁴ together with the carbon to which they are attached form a group selected from aryl, substituted aryl, heteroaryl, substituted heteroaryl, heterocycle, substituted heterocycle, cycloalkyl and substituted cycloalkyl;
R⁷ and R⁸ are each independently selected from H, alkyl, substituted alkyl, alkoxy, substituted alkoxy, halogen, CF₃, carboxy, phosphonate, carboxylate, cyano, hydroxyl, carboxyamide, sulfonamide, sulfonate, amino, substituted amino, amide, aryl, substituted aryl, heteroaryl, substituted heteroaryl, heterocycle and substituted heterocycle;
or R⁷ and R⁸ together with the carbon to which they are attached form a group selected from aryl, substituted aryl, heteroaryl, substituted heteroaryl, heterocycle, substituted heterocycle, cycloalkyl and substituted cycloalkyl; and
R⁵, R⁶, R⁹ and R¹⁰ are each independently selected from H, alkyl, substituted alkyl, alkoxy, substituted alkoxy, halogen, CF₃, carboxy, phosphonate, carboxylate, cyano, hydroxyl, carboxyamide, sulfonamide, sulfonate, amino, substituted amino, amide, aryl, substituted aryl, heteroaryl, substituted heteroaryl, heterocycle and substituted heterocycle;
T is an integer from 0 to 1;
q is an integer from 1 to 20;
R²² is selected from H, alkyl, substituted alkyl, aryl, substituted aryl, heterocycle, substituted heterocycle, heteroaryl, substituted heteroaryl;
R¹⁶, R¹⁷, R¹⁸ and R¹⁹ are each independently selected from H, alkyl, substituted alkyl, alkoxy, substituted alkoxy, halogen, CF₃, sulfonate, amino, substituted amino, amide, aryl, substituted aryl, heteroaryl, substituted heteroaryl, heterocycle and substituted heterocycle; and
X³ is selected from a heteroatom, a substituted heteroatom, CH₂ and a substituted carbon atom.

Clause 33. The functional dye of claim 32, of structure (10):

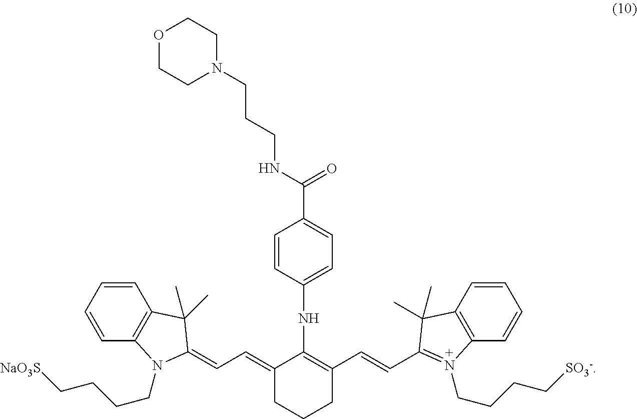

Clause 34. The functional dye of any one of clauses 1 to 3, wherein the functional dye is of the formula (A2):

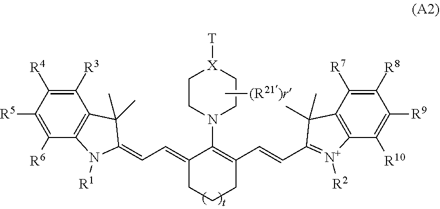

wherein:
R¹ and R² are each independently selected from H, alkyl, and (CH₂)ₙR¹², wherein n is an integer from 1 to 20 and R¹² is selected from the group consisting of H, sulfonate, carboxylic acid, amine, quaternary ammonium cation, phosphate, ester, halogen, azide, cyano, alkyne, and heterocycle;
R³ and R⁴, are each independently selected from H, alkyl, substituted alkyl, alkoxy, substituted alkoxy, halogen, CF₃, carboxy, phosphonate, carboxylate, cyano, hydroxyl, carboxyamide, sulfonamide, sulfonate, amino, substituted amino, amide, aryl, substituted aryl, heteroaryl, substituted heteroaryl, heterocycle and substituted heterocycle;
or R³ and R⁴ together with the carbon to which they are attached form a group selected from aryl, substituted aryl, heteroaryl, substituted heteroaryl, heterocycle, substituted heterocycle, cycloalkyl and substituted cycloalkyl;
R⁷ and R⁸ are each independently selected from H, alkyl, substituted alkyl, alkoxy, substituted alkoxy, halogen, CF₃, carboxy, phosphonate, carboxylate, cyano, hydroxyl, carboxyamide, sulfonamide, sulfonate, amino, substituted amino, amide, aryl, substituted aryl, heteroaryl, substituted heteroaryl, heterocycle and substituted heterocycle;
or R⁷ and R⁸ together with the carbon to which they are attached form a group selected from aryl, substituted aryl, heteroaryl, substituted heteroaryl, heterocycle, substituted heterocycle, cycloalkyl and substituted cycloalkyl; and
R⁵, R⁶, R⁹ and R¹⁰ are each independently selected from H, alkyl, substituted alkyl, alkoxy, substituted alkoxy, halogen, CF₃, carboxy, phosphonate, carboxylate, cyano, hydroxyl, carboxyamide, sulfonamide, sulfonate, amino, substituted amino, amide, aryl, substituted aryl, heteroaryl, substituted heteroaryl, heterocycle and substituted heterocycle;
t is an integer from 0 to 1;
X is selected from a N, CH and CR³², wherein R³² is selected from alkyl, substituted alkyl, hydroxy, alkoxy, substituted alkoxy, halogen, CF₃, sulfonate, amino, substituted amino, amide, aryl, substituted aryl, heteroaryl, substituted heteroaryl, heterocycle and substituted heterocycle;
T is a protein targeting moiety comprising a fragment of a known drug that targets a protein selected from the group consisting of (non)-receptor kinases, immune checkpoint proteins, G-protein-coupled receptors (GPCRs), influx transporters, efflux transporters, ion channels, human leukocyte antigens (HLA), proteases, caspases and nuclear receptors from a small molecule kinase inhibitor or a fragment of a small molecule modulator of GPCRs;
R^{21′} are each independently selected from alkyl, substituted alkyl, alkoxy, substituted alkoxy, halogen, CF₃, sulfonate, amino, substituted amino, amide, aryl, substituted aryl, heteroaryl, substituted heteroaryl, heterocycle and substituted heterocycle; and
r′ is an integer from 0 to 8.

Clause 35. The functional dye of claim 34, wherein T is a fragment of imatinib, bosutinib, nintedanib, ponatinib, brigatinib, dasatinib or palbociclib.

Clause 36. The functional dye of clause 34, wherein T is a fragment of sildenafil or eszopiclone.

Clause 37. The functional dye of clause 34, wherein T is selected from:

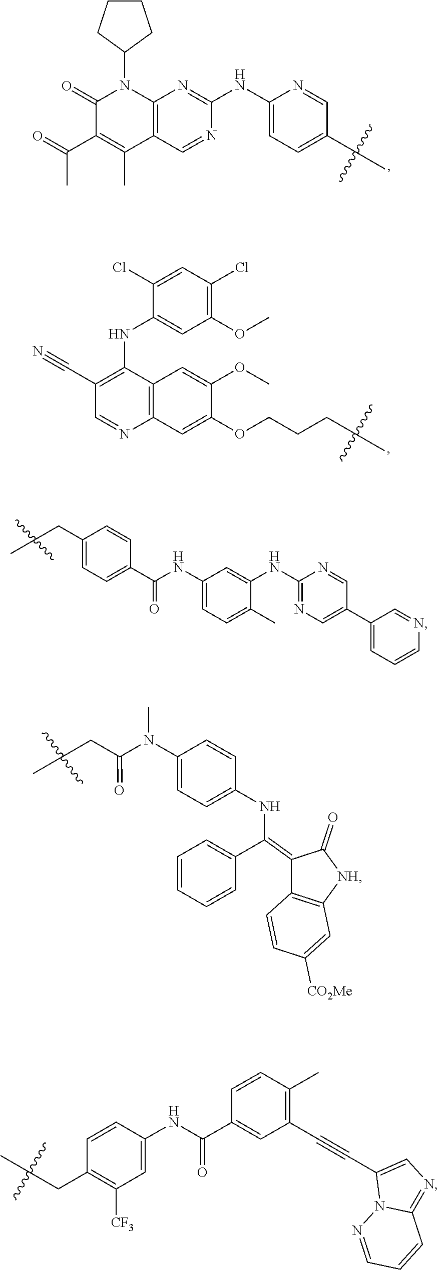 [SMILES: *c1ccc(Nc2ncc3c(C)c(C(C)=O)c(=O)n(C4CCCC4)c3n2)nc1 ; *CCCOc1cc2ncc(C#N)c(Nc3cc(OC)c(Cl)cc3Cl)c2cc1OC ; *Cc1ccc(C(=O)Nc2ccc(C)c(Nc3ncc(-c4cccnc4)cn3)c2)cc1 ; *CC(=O)N(C)c1ccc(N/C(=C2\C(=O)Nc3cc(C(=O)OC)ccc32)c2ccccc2)cc1 ; *Cc1ccc(NC(=O)c2ccc(C)c(C#Cc3cnc4cccnn34)c2)cc1C(F)(F)F]

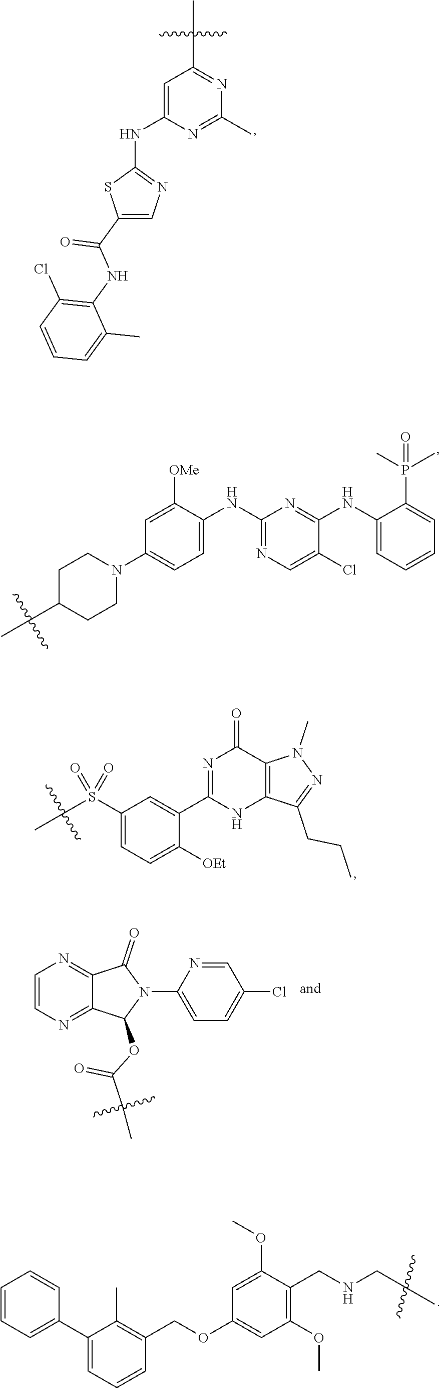 [SMILES: *c1cc(Nc2ncc(C(=O)Nc3c(C)cccc3Cl)s2)nc(C)n1 ; *C1CCN(c2ccc(Nc3ncc(Cl)c(Nc4ccccc4P(C)(C)=O)n3)c(OC)c2)CC1 ; *S(=O)(=O)c1ccc(OCC)c(-c2nc(=O)c3c([nH]2)c(CCC)nn3C)c1 ; *C(=O)O[C@H]1c2nccnc2C(=O)N1c1ccc(Cl)cn1 ; *CNCc1c(OC)cc(OCc2cccc(-c3ccccc3)c2C)cc1OC]

Clause 38. A pharmaceutical composition, comprising: a functional dye of any one of clauses 1-37, and a pharmaceutically acceptable excipient.

Clause 39. A pharmaceutical composition for use as an oral contrast agent, comprising: a functional dye of any one of clauses 1-37; and a pharmaceutically acceptable excipient.

Clause 40. The pharmaceutical composition of clauses 38 or 39, further comprising one or more agents selected from PEG and sodium bicarbonate.

Clause 41. The pharmaceutical composition of clause 40, wherein the PEG is PEG3350.

Clause 42. A method of imaging a tumor, the method comprising: administering a pharmaceutical composition according to any one of clauses 38 to 40 to an individual; detecting the presence of the functional dye; wherein increased concentration of the dye is indicative of a lesion.

Clause 43. The method of clause 42, wherein the functional dye is detected with a fluorescence endoscopic camera or a fluorescence bronchoscopic camera.

Clause 44. The method of clause 42, wherein the functional dye is detected with a fluorescence capsule endoscopic camera, a cystoscope or a laparoscope.

Clause 45. The method of clause 43 or 44 wherein the camera is a combined white light near-infrared (WL/NIRF) camera.

Clause 46. The method of any one of clauses 42 to 45, wherein the lesion is a cancerous or precancerous lesion.

Clause 47. The method of clause 46, wherein the lesion is found in one or more locations selected from the gastrointestinal tract, breast, brain, prostate, pancreas, skin, bladder, head, neck and thyroid.

Clause 48. The method of any one of claims 42 to 47, wherein administration of the functional dye is oral.

Clause 49. The method of any one of claims 42 to 48, wherein detection of the dye is used to guide biopsy or surgical resection of the lesion.

Clause 50. A kit comprising: a functional dye of any one of clauses 1-37; and a device for detecting the presence of the functional dye.

Clause 51. The kit of clause 50, wherein the device for detection of the functional dye is selected from a capsule endoscope, a portable cytometer, a smartphone add on (e.g., combined NIRF/WL camera unit) and a wearable cytometer.

### Additional Embodiments A

Clause 1A. A functional dye of the formula:

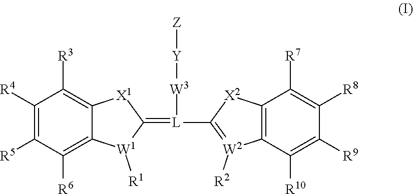

wherein:
L is a polymethine group or a substituted polymethine group;
R¹ and R² are each independently selected from H, alkyl, and (CH₂)ₙR¹², wherein n is an integer from 1 to 20 and R¹² is selected from the group consisting of sulfonate, carboxylic acid, amine, quaternary ammonium cation, phosphate, ester, halogen, azide, cyano, alkyne, and heterocycle;
R³ and R⁴, are each independently selected from H, alkyl, substituted alkyl, alkoxy, substituted alkoxy, halogen, sulfonate, amino, substituted amino, amide, aryl, substituted aryl, heterocycle and substituted heterocycle;
or R³ and R⁴ together with the carbon to which they are attached form a group selected from aryl, substituted aryl, heterocycle, substituted heterocycle, cycloalkyl and substituted cycloalkyl;
R⁷ and R⁸ are each independently selected from H, alkyl, substituted alkyl, alkoxy, substituted alkoxy, halogen, sulfonate, amino, substituted amino, amide, aryl, substituted aryl, heterocycle and substituted heterocycle;
or R⁷ and R⁸ together with the carbon to which they are attached form a group selected from aryl, substituted aryl, heterocycle, substituted heterocycle, cycloalkyl and substituted cycloalkyl;
R⁵, R⁸, R⁹ and R¹⁰ are each independently selected from H, alkyl, substituted alkyl, alkoxy, substituted alkoxy, halogen, sulfonate, amino, substituted amino, amide, aryl, substituted aryl, heterocycle and substituted heterocycle;
X¹ and X² are each independently selected from a heteroatom, CH₂, C(R¹³)₂, wherein each R¹³ is independently selected from H, alkyl and substituted alkyl;
W¹ is selected from a heteroatom or CH₂;
W² is selected from a cationic heteroatom;
W³ is selected from a heteroatom, NR¹¹ or C(R¹¹)₂, wherein R¹¹ is selected from H or alkyl;
Y is selected from the group consisting of alkyl, substituted alkyl, aryl, substituted aryl, carboxyl and substituted carboxyl; and
Z is selected from the group consisting of substituted amine, morpholine, substituted morpholine, thiomorpholine, substituted thiomorpholine, piperazine and substituted piperazine;
or a (pro-)drug, a pharmaceutically acceptable salt or a solvate thereof.

Clause 2A. The functional dye of clause 1A, wherein L is selected from:

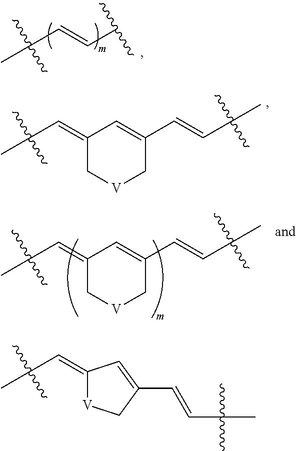

wherein, m is an integer from 1 to 4; and
V is selected from a heteroatom, C(R²¹)₂, wherein each R²¹ is independently selected from H or an alkyl group.

Clause 3A. The functional dye of clause 2A, wherein L is:

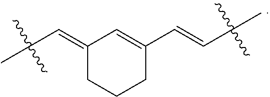 [SMILES: */C=C/C1=CC(=C/*)/CCC1]

Clause 4A. The functional dye of any one of clauses 1A to 3A, wherein R¹ and R² are both (CH₂)ₙR¹², wherein n is 3 and R¹² is sulfonate or a salt thereof.

Clause 5A. The functional dye of any one of clauses 1A-4A, wherein X¹ and X² are both C(R¹³)₂, wherein R¹³ is methyl.

Clause 6A. The functional dye of any one of clauses 1A-5A, wherein each of R³ to R¹⁰ is H.

Clause 7A. The functional dye of clause 1A, of the formula:

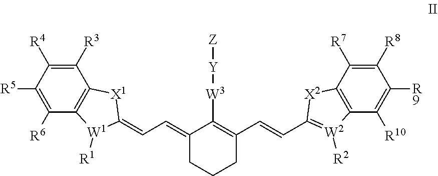

wherein:
R¹ and R² are each independently selected from H, alkyl, and (CH₂)ₙR¹², wherein n is an integer from 1 to 20 and R¹² is selected from the group consisting of sulfonate, carboxylic acid, amine, quaternary ammonium cation, phosphate, ester, halogen, azide, cyano, alkyne, and heterocycle;
R³ and R⁴, are each independently selected from H, alkyl, substituted alkyl, alkoxy, substituted alkoxy, halogen, sulfonate, amino, substituted amino, amide, aryl, substituted aryl, heterocycle and substituted heterocycle;
or R³ and R⁴ together with the carbon to which they are attached form a group selected from aryl, substituted aryl, heterocycle, substituted heterocycle, cycloalkyl and substituted cycloalkyl;
R⁷ and R⁸ are each independently selected from H, alkyl, substituted alkyl, alkoxy, substituted alkoxy, halogen, sulfonate, amino, substituted amino, amide, aryl, substituted aryl, heterocycle and substituted heterocycle;
or R⁷ and R⁸ together with the carbon to which they are attached form a group selected from aryl, substituted aryl, heterocycle, substituted heterocycle, cycloalkyl and substituted cycloalkyl;
R⁵, R⁶, R⁹ and R¹⁰ are each independently selected from H, alkyl, substituted alkyl, alkoxy, substituted alkoxy, halogen, sulfonate, amino, substituted amino, amide, aryl, substituted aryl, heterocycle and substituted heterocycle;
X¹ and X² are each independently selected from a heteroatom, CH₂, C(R¹³)₂, wherein each R¹³ is independently selected from H, alkyl and substituted alkyl;
W¹ is selected from a heteroatom or CH₂;
W² is selected from a cationic heteroatom;
W³ is selected from a heteroatom, NR¹¹ or C(R¹¹)₂, wherein R¹¹ is selected from H or alkyl;
Y is selected from the group consisting of alkyl, substituted alkyl, aryl, substituted aryl, carboxyl and substituted carboxyl; and
Z is selected from the group consisting of substituted amine, morpholine, substituted morpholine, thiomorpholine, substituted thiomorpholine, piperazine and substituted piperazine;
or a pro-drug, a pharmaceutically acceptable salt or a solvate thereof.

Clause 8A. The functional dye of clause 7A, of the formula:

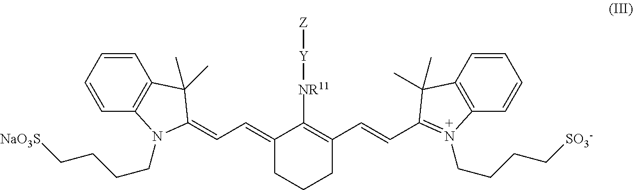

wherein:
R¹¹ is selected from H or alkyl;
Y is selected from the group consisting of alkyl, substituted alkyl, aryl, substituted aryl, carboxyl and substituted carboxyl;
Z is selected from the group consisting of substituted amine, morpholine, substituted morpholine, thiomorpholine, substituted thiomorpholine, piperazine and substituted piperazine;
or a pro-drug, a pharmaceutically acceptable salt or a solvate thereof.

Clause 9A. The functional dye of any one of clauses 1A-8A, wherein Y is selected from:

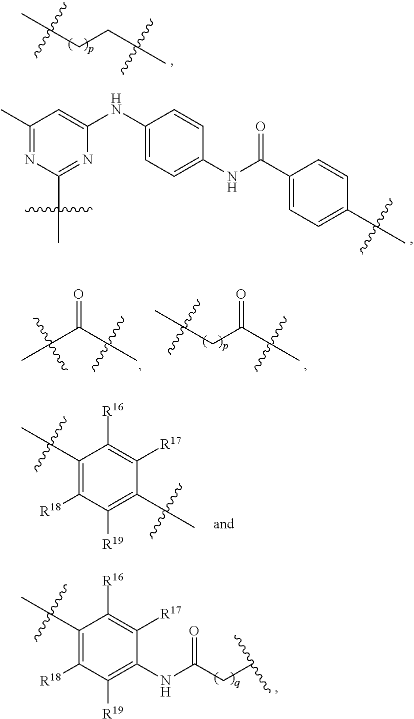

wherein:
each p is independently an integer from 1 to 20;
q is an integer from 1 to 20;
R¹⁶, R¹⁷, R¹⁸ and R¹⁹ are each independently selected from H, alkyl, substituted alkyl, alkoxy, substituted alkoxy, halogen, sulfonate, amino, substituted amino, amide, aryl, substituted aryl, heterocycle and substituted heterocycle.

Clause 10A. The functional dye of any one of clauses 1A to 9A, wherein Z is selected from:

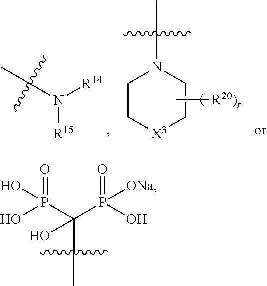

wherein:
R¹⁴ and R¹⁵ are each independently selected from C₁₋₆ alkyl;
R²⁰ is a substituent selected from alkyl, substituted alkyl, alkoxy, substituted alkoxy, halogen, sulfonate, amino, substituted amino, amide, aryl, substituted aryl, heterocycle and substituted heterocycle;
r is an integer from 0 to 4; and
X³ is selected from O, S, N and C.

Clause 11A. The functional dye of any one of clauses 1A-10A, wherein Y is aryl or substituted aryl.

Clause 12A. The functional dye of any one of clauses 1A-11A, wherein Z is morpholine or substituted morpholine.

Clause 13A. The functional dye of any one of clauses 1A-10A, wherein NR¹¹—Y—Z together form a group selected from:

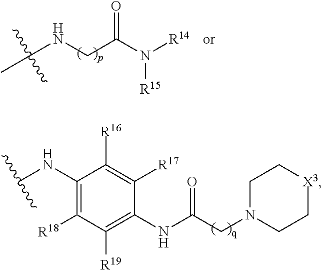

wherein:
p is an integer from 0 to 20;
R¹⁴ and R¹⁵ are each independently selected from C₁₋₆ alkyl;
q is an integer from 1 to 20;
R¹⁶, R¹⁷, R¹⁸ and R¹⁹ are each independently selected from H, alkyl, substituted alkyl, alkoxy, substituted alkoxy, halogen, sulfonate, amino, substituted amino, amide, aryl, substituted aryl, heterocycle and substituted heterocycle; and
X³ is selected from O, S, N and C.

Clause 14A. The functional dye of claim 13A, wherein NR¹¹—Y—Z together form:

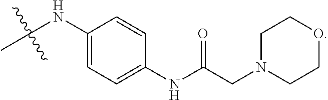 [SMILES: *Nc1ccc(NC(=O)CN2CCOCC2)cc1]

Clause 15A. The functional dye of any one of clauses 1A to 14A selected from the following structures:

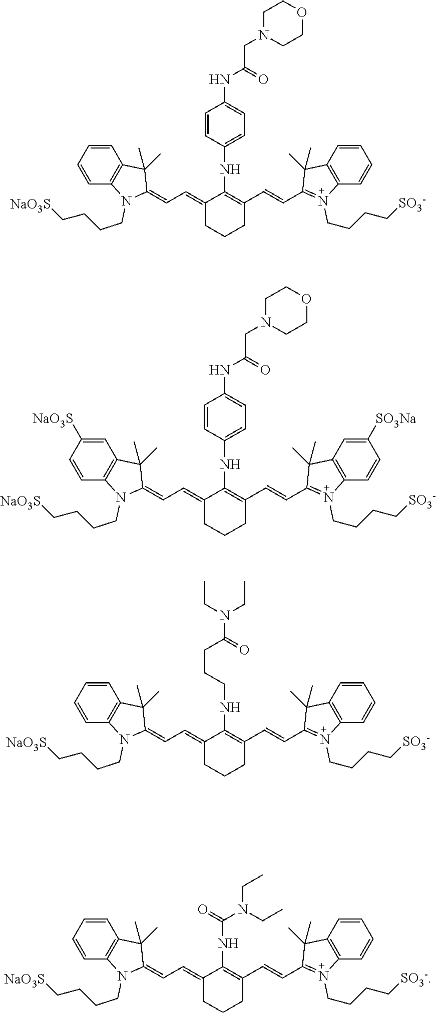

Clause 16A. A pharmaceutical composition, comprising: a functional dye of any one of clauses 1A-15A; and a pharmaceutically acceptable excipient.

Clause 17A. A pharmaceutical composition for use as an oral contrast agent, comprising: a functional dye of any one of clauses 1A-15A, and a pharmaceutically acceptable excipient,

Clause 18A. The pharmaceutical composition of clause 16A or 17A, further comprising one or more agents selected from PEG and sodium bicarbonate.

Clause 19A. The pharmaceutical composition of clause 18A, wherein the PEG is PEG3350.

Clause 20A. A method of imaging a tumor, the method comprising: administering a pharmaceutical composition according to clause 16A or 17A to an individual; detecting the presence of the functional dye; wherein increased concentration of the dye is indicative of a lesion.

Clause 21A. The method of clause 20A, wherein the functional dye is detected with a fluorescent endoscopic camera.

Clause 22A. The method of clause 20A, wherein the lesion is a cancerous or precancerous lesion of the gastrointestinal tract.

Clause 23A. The method of clause 20A, wherein administration of the functional dye is oral.

Clause 24A. The method of clause 20A, wherein detection of the dye is used to guide surgery of the lesion.

### Experimental

The small molecular compound SU-783 (also referred to herein as compound (1)) was identified using a fragment-based screen against high-grade adenomas in intestinal tissues of Apcm^{Min/+}—a preclinical model that closely recapitulates colorectal carcinogenesis in humans_{2,3}. SU-783 consists of a clinically-applied near-infrared fluorescent (NIRF) dye-backbone (i.e. IR783) conjugated to an adenoma-targeting moiety that is equipped with a morpholine functional group to ensure intracellular entrapment following uptake by tumor-associated cells.

### Example 1: Synthesis of Exemplary Functional Dyes

General Procedure:

A near infrared dye (e.g., IR780, IR783, S0456, S2493 etc.) is conjugated to terminal group of a subject drug or drug fragment, (e.g., an amine, thiol, hydroxy, piperidine or piperazine terminal group, or a synthetic equivalent thereof) in a polar aprotic solvent (e.g., N,N-dimethylformamide (DMF), dimethylsulfoxide (DMSO), etc.) and heating overnight (e.g., at 60-100° C., such as 70° C.), optionally in the presence of a catalytic amount of base (e.g., triethylamine, diisopropylethylamine, etc.).

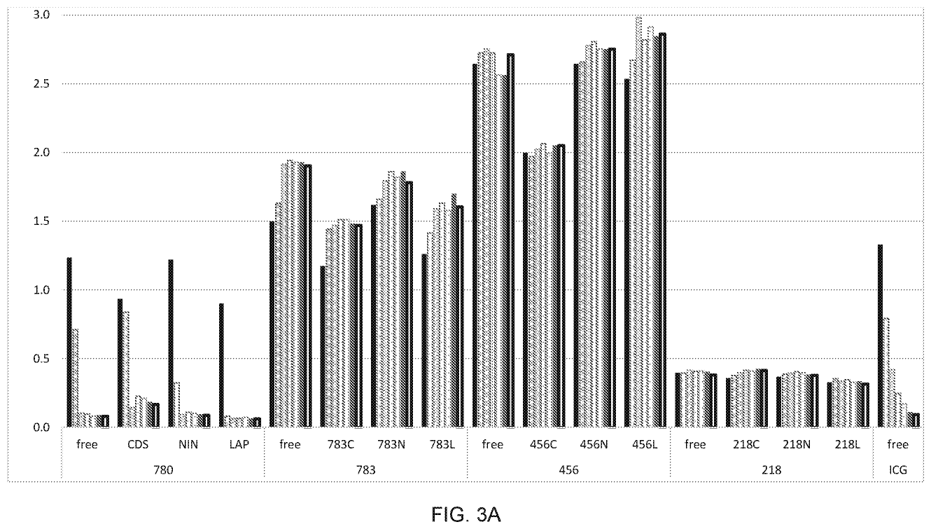

Exemplary compound SU-783 (Compound (1)) was synthesized according to the scheme illustrated in FIG. 2. The subject compounds may be synthesized in one step from commercially available materials without the need for further purification. An exemplary procedure, as illustrated in FIG. 2, is as follows: equimolar amounts of IR-783 (Sigma Aldrich, 543292) and N-(4-aminophenyl)-2-morpholin-4-ylacetamide (Sigma Aldrich, CDS004865) in dimethylformamide (DMF) were stirred overnight at 72° C. After which time, compound (1) was obtained and used in the applications described herein without further purification.

Functional Dye Stability in Water:

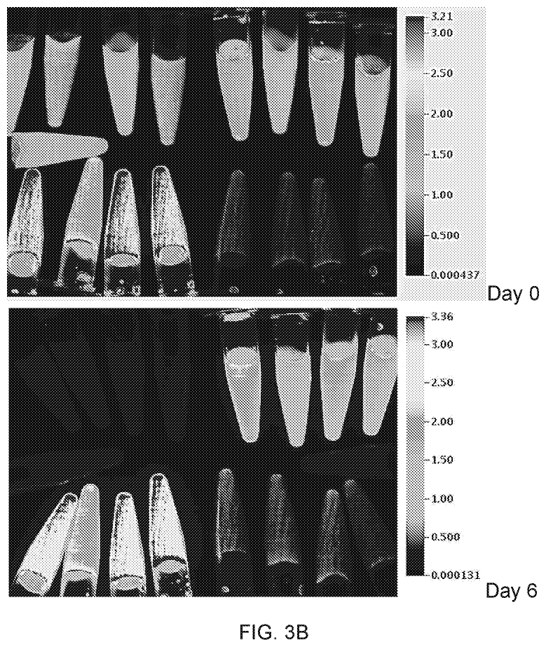

Compound (1), and other exemplary compounds, were dissolved in water at a concentration of 25 M and were subjected to near infrared fluorescence (λₑₓ 785 nm; λₑₘ>800 nm) every day for a week. As shown in FIG. 3A and FIG. 3B, compared to the current clinical standard indocyanine green (ICG), the functional dyes derived from IR783, S0456, and S2180 produced a stable fluorescent signal for the indicated time period. Furthermore, when NIR-II fluorescence imaging (λₑₓ 790 nm; λₑₘ>1000 nm) was performed after 2 weeks in water, the functional derived from S0456 and S2180 were still fluorescent and produced higher fluorescence intensity than a freshly prepared solution of an equimolar amount of ICG (FIG. 3C).

### Example 2: Detection of Adenomas in a Mouse Model of Intestinal Carcinogenesis Following Oral Administration of an Exemplary Non-Systemic Functional Dye (Compound (1), SU783)

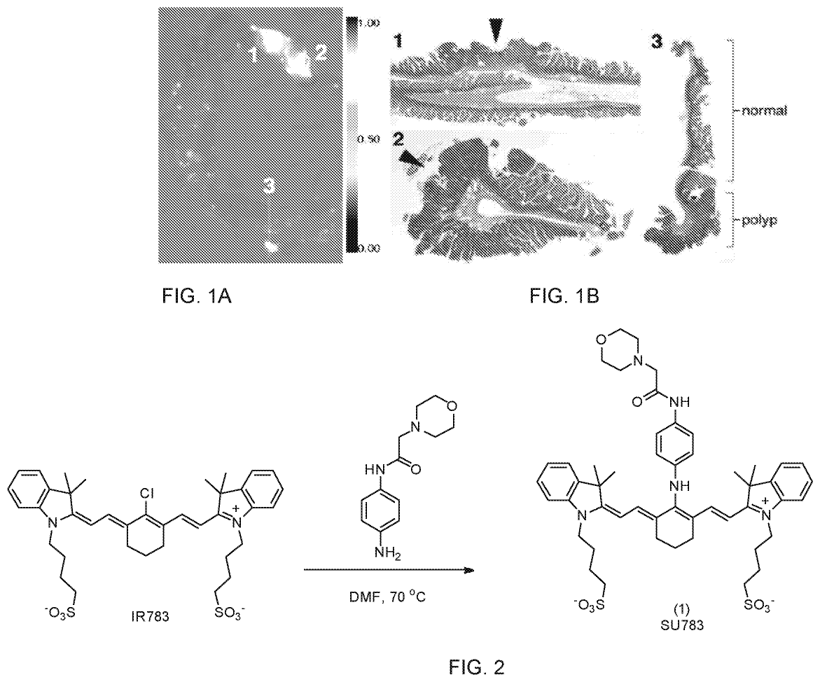

Apc^{Min/+} mice (14-20-week old; female; n=5) received an oral gavage of Compound (1) (SU-783) (0.4 mg/kg) in 8.4% sodium bicarbonate. The next day, the animals were sacrificed, the small intestines were harvested, washed with phosphate buffered saline (PBS), and imaged using NIRF (λₑₓ 785 nm; λₑₘ>800 nm). As shown in FIG. 1B to FIG. 1C, following oral administration of SU783, it highlighted adenomas throughout the intestinal tract with tumor-to-background ratios (TBR) >5. Furthermore, since the functional dye was designed to violate the Lipinski rule of 5, it was expected and observed that following oral administration SU783 was not taken up systemically and remained exclusively in the alimentary tract.

A freshly excised colon of an Apc^{Min/+} mouse was submerged in 100 microliters of freshly prepared 2.5 micromolar solution of SU-783 (Compound (1)) in 5% (w/v) D-glucose in water (D5W). After 15 min, the colon was washed three times with excessive D5W and imaged on a near-infrared fluorescence imager using 785-nm laser excitation.

To achieve the highest tumor/background ratio (TBR), SU-783 was designed to have strong fluorescent emission in the near-infrared (>800 nm) range where tissue autofluorescence is minimal. Finally, with a polar surface area >140 Å (i.e. 176 Å) and >10 rotatable bonds (i.e. 16), theoretical considerations predict a poor oral availability₄, which supports the oral application of SU-783 as a non-systemic contrast agent. Peclinical evaluation of orally administered SU-783 in Apc^{Min/+} mice demonstrated sensitive detection of adenomatous polyps with TBRs (FIG. 1A-1B) that were equivalent or better than those of clinically-approved, systemically-administered optical contrast agents such as IRdye800CW-labeled cetuximab (TBR 5.2) or OTL38 (TBR 4.4).

SU-783 is an oral contrast agent developed to improve the adenoma detection rate (ADR) and diagnostic accuracy for CRC and high-grade dysplasia—the most clinically relevant CRC precursor lesion₇—during colonoscopic surveillance of the lower GI tract. CRC is the second most lethal cancer worldwide and it has been shown that colonoscopic screening programs contributed to reduction of CRC incidence and mortality by 83% and 89%, respectively, due to timely detection and removal of high-grade premalignant polyps (i.e. incipient CRC), which cause ˜80% of CRCs₈. Currently, an estimated 11 million colonoscopies were performed in the US in 2012. Even when fecal immunochemical testing (FIT)—a noninvasive CRC test—is implemented on a large-scale, it is predicted that by 2024 the number of colonoscopies in the US will still be on the order of 5-16 million on an annual basis.

Shown in FIG. 1 is wide-field near-infrared fluorescence imaging (I_{ex,em}=785, >800 nm) of intestinal section of an Apcm^{Min/+} mouse 5 h after oral administration of SU-783 at a dose of 0.4 mg/kg (in 8.4% bicarbonate). Lesions 1 (TBR=6.1), 2 (TBR=5.3), and 3 (TBR=5.2) were sectioned and processed for histopathological examination by a veterinary pathologist. All NIRF-positive lesions were identified as sessile adenomas (arrow heads). The sensitivity and specificity is particularly well-illustrated by tissue section 3, where SU-783 discretely highlights the sessile adenoma (high fluorescence signal) without staining normal mucosa. Of note, following oral administration, no SU-783-associated fluorescence signal was found in the blood, liver, kidneys, or bladder indicating that SU-783 was not systemically absorbed.

### Example 3: Detection of Adenomas in a Rat Model of Colorectal Carcinogenesis Following Oral Administration of an Exemplary Non-Systemic Functional Dye (Compound (1), SU783)

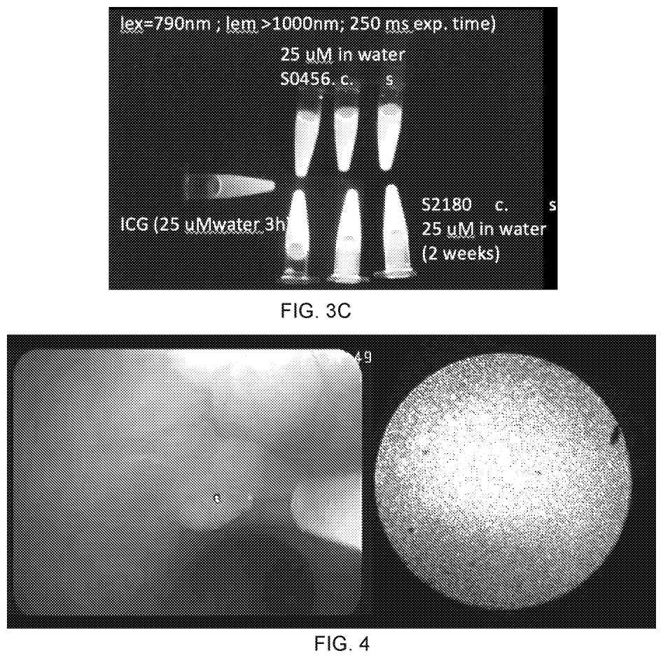

Apc^{Pirc/+} rats (14-20-week old; male/female; n=5) received an oral gavage of Compound (1) (SU783) (0.4 mg/kg) in 8.4% sodium bicarbonate. The next day, the animals were anesthetized using isoflurane (2% v/v), their colons washed with PBS, and NIRF endoscopy (λₑₓ 785 nm; λₑₘ>800 nm) was performed. As shown in FIG. 4, exemplary Compound (1) highlighted adenomas in real-time during NIRF endoscopy.

### Example 4: Affinity of Functional Dye for Human Adenomas Versus Healthy, Normal Colorectal Tissues

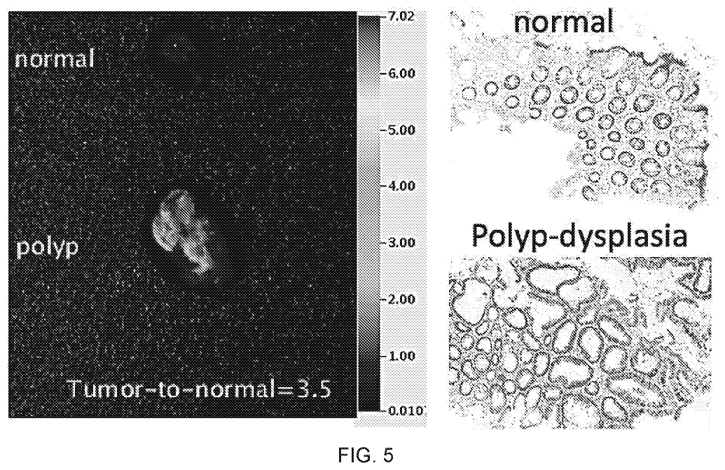

Freshly-resected human biopsies were incubated in a solution of 12.5 μM Compound (1) (SU783) in PBS for 15 min at 37′C. The washed tissue biopsies were subjected to NIRF imaging and it was shown that Compound (1) exhibits a 3.5× higher tumor-over-normal signal (FIG. 5).

### Example 5: Detection of Breast Cancer Following Oral Administration of an Exemplary Systemic Functional Dye (Compound (8), SU 780)

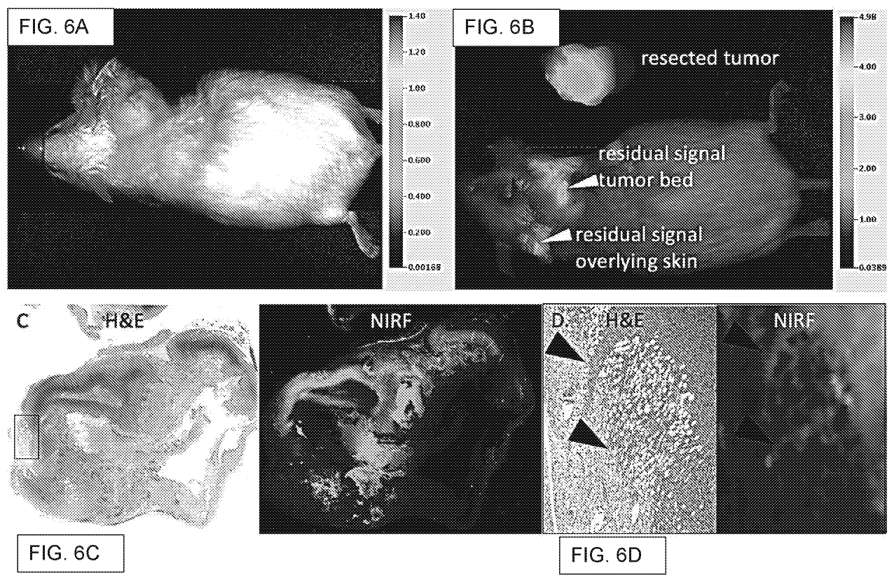

A wild type with an unidentified spontaneous flank tumor received an oral gavage of Compound (8) (SU780) (0.4 mg/kg). The following day, the animal was sacrificed, and the tumor was subjected to NIRF imaging. As shown in FIG. 6B, the tumor was highlighted by Compound (1). Furthermore, when NIRF imaging was performed on tissue section of the tumor, it showed perfect correlation with tumor infiltration into normal tissues.

Shown in FIG. 6 is the highlighting of a breast tumor following oral administration of the systemic functional dye Compound (8) (SU780). FIG. 6A shows NIRF imaging of the intact mouse 24 h post oral gavage of Compound (8). FIG. 6B shows NIRF imaging of resected tumor, demonstrating that SU780 highlights the tumor following oral administration. Furthermore, FIG. 6B shows that Compound (8) can be used during image-guided resection since residual tumor tissue was fluorescing in the resection bed. FIG. 6C shows H&E stained tumor section of the tumor, demonstrating this was a breast cancer. Interestingly, after tissue processing for H&E, SU780 is still present in the section and highlights mainly epithelial cancer cells. FIG. 6D shows higher magnification of tumor margin (black square in C) demonstrating that SU780 highlights the finger-like infiltration of the breast cancer cells into normal mammary fat pad (arrow heads).

White-light endoscopy currently is the mainstay for (incipient) CRC screening. However, multiple studies have reported that 17-28% of colorectal polyps are missed. Particularly, the miss-rate of 32.7% for flat or sessile polyps—lesions that are 5 times more likely to be cancerous relative to pedunculated polyps—is significant. Moreover, it is often difficult to determine the true lateral extent of these lesions, thus hampering the ability to achieve complete endoscopic mucosal resection (EMR). Consequently, approximately 15-26% of CRC recur at or near the therapeutic site.

There is an unmet need for novel, minimally invasive-imaging approaches that reliably enable highly sensitive and specific detection of (pre)malignant colorectal lesions. While targeted biopsy using dyes to delineate mucosal abnormalities (i.e. chromoendoscopy) has a higher detection rate, it is not embraced by endoscopists due to the perceived difficulty, cost, and time associated with intraluminal dye administration₁. To mitigate the high miss-rate and low diagnostic accuracy of conventional white-light endoscopy, and, negate the perceived drawbacks of chromoendoscopy, the functional dyes aim at the clinical implementation of a strategy to orally administer the NIRF dye SU-783 prior to endoscopic surveillance to highlight high-grade lesions during endoscopy and improve targeted biopsy and endotherapeutic intervention.

What is claimed is:

1. A functional dye of formula:
D-L-F
wherein:
D is a near infrared fluorescent dye;
L is an optional linker; and
F is a small molecule that targets a protein.

2. The functional dye of claim 1, wherein the small molecule is a known drug, or a fragment of a known drug that targets a protein selected from the group consisting of (non)-receptor kinases, immune checkpoint proteins, G-protein-coupled receptors (GPCRs), influx transporters, efflux transporters, ion channels, human leukocyte antigens (HLA), proteases, caspases and nuclear receptors.

3. The functional dye of claim 1 or 2, wherein the protein is selected from a wild type, a splice variant,

4. The functional dye of any one of claims 1 to 3, wherein F comprises a small molecule kinase inhibitor or a small molecule kinase inhibitor fragment.

5. The functional dye of claim 4, wherein the small molecule kinase inhibitor is selected from ceritinib and palbociclib.

6. The functional dye of claim 4, wherein the small molecule kinase inhibitor fragment is selected from N-desmethyl imatinib, N-desmethyl bosutinib, N-desmethyl nintedanib, N-desmethyl ponatinib, N-desmethyl brigatinib, N-deshydroxyethyl dasatinib.

7. The functional dye of claim 4, wherein the small molecule kinase inhibitor fragment comprises a fragment of nintedanib or lapatinib.

8. The functional dye of any one of claims 1 to 3, wherein F comprises a small molecule modulator of G-protein-coupled receptors (GPCRs) or a fragment of a small molecule modulator of G-protein-coupled receptors (GPCRs).

9. The functional dye of claim 8, wherein the small molecule modulator of G-protein-coupled receptors (GPCRs) is selected from N-desmethyl sildenafil and N-desmethyl eszopiclone.

10. The functional dye of any one of claims 1 to 3, wherein F comprises a small molecule agonist of ion-channels.

11. The functional dye of claim 10, wherein the small molecule agonist of ion-channels is procainamide.

12. The functional dye of claim 1, wherein F comprises a small molecule of the following structure:

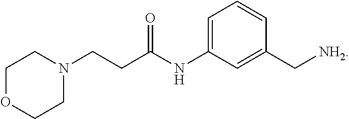 [SMILES: NCc1cccc(NC(=O)CCN2CCOCC2)c1]

13. The functional dye of any one of claims 1 to 3, wherein F is selected from a fragment of formula (F1) and a fragment of formula (F2), wherein fragment (F1) is of the formula: 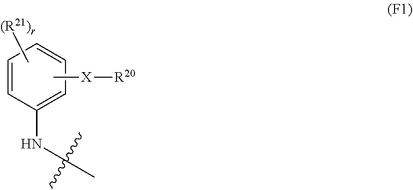
wherein:
X is selected from C(O)NR²², NR²², O and S, wherein R²² is selected from H, alkyl, substituted alkyl, aryl, substituted aryl, heterocycle, substituted heterocycle, heteroaryl and substituted heteroaryl;
R²⁰ is selected from substituted alkyl, substituted acyl, benzyl, substituted benzyl, aryl, substituted aryl, heteroaryl, substituted heteroaryl, heterocycle, substituted heterocycle;
R²¹ are each independently selected from alkyl, substituted alkyl, alkoxy, substituted alkoxy, halogen, CF₃, sulfonate, amino, substituted amino, amide, aryl, substituted aryl, heteroaryl, substituted heteroaryl, heterocycle and substituted heterocycle; and
r is an integer from 0 to 4; and
fragment (F2) is of the formula 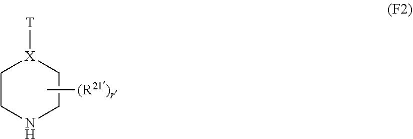
wherein:
X is selected from a N, CH and CR³², wherein R³² is selected from alkyl, substituted alkyl, hydroxy, alkoxy, substituted alkoxy, halogen, CF₃, sulfonate, amino, substituted amino, amide, aryl, substituted aryl, heteroaryl, substituted heteroaryl, heterocycle and substituted heterocycle;
T is a protein-targeting moiety comprising a fragment of a known drug that targets a protein selected from the group consisting of (non)-receptor kinases, immune checkpoint proteins, G-protein-coupled receptors (GPCRs), influx transporters, efflux transporters, ion channels, human leukocyte antigens (HLA), proteases, caspases and nuclear receptors from a small molecule kinase inhibitor or a fragment of a small molecule modulator of GPCRs;
R^{21′} are each independently selected from alkyl, substituted alkyl, alkoxy, substituted alkoxy, halogen, CF₃, sulfonate, amino, substituted amino, amide, aryl, substituted aryl, heteroaryl, substituted heteroaryl, heterocycle and substituted heterocycle; and
r′ is an integer from 0 to 8.

14. The functional dye of claim 13, wherein fragment (F1) is of the formula (F1A): 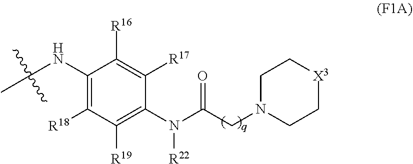
wherein:
q is an integer from 1 to 20;
R²² is selected from H, alkyl, substituted alkyl, aryl, substituted aryl, heterocycle, substituted heterocycle, heteroaryl, substituted heteroaryl;
R¹⁶, R¹⁷, R¹⁸ and R¹⁹ are each independently selected from H, alkyl, substituted alkyl, alkoxy, substituted alkoxy, halogen, CF³, sulfonate, amino, substituted amino, amide, aryl, substituted aryl, heteroaryl, substituted heteroaryl, heterocycle and substituted heterocycle; and
X³ is selected from a heteroatom, a substituted heteroatom, CH₂ and a substituted carbon atom.

15. The functional dye of claim 14, wherein formula (F1A) is of the structure:

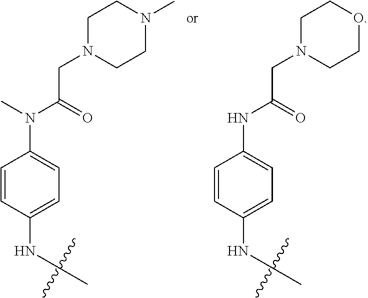

16. The functional dye of claim 13, wherein fragment (F1) is of the formula (F1B): 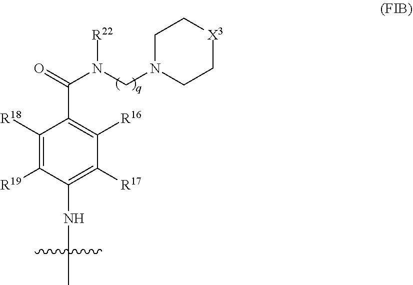
wherein:
q is an integer from 1 to 20;
R²² is selected from H, alkyl, substituted alkyl, aryl, substituted aryl, heterocycle, substituted heterocycle, heteroaryl, substituted heteroaryl;
R¹⁶, R¹⁷, R¹⁸ and R¹⁹ are each independently selected from H, alkyl, substituted alkyl, alkoxy, substituted alkoxy, halogen, CF³, sulfonate, amino, substituted amino, amide, aryl, substituted aryl, heteroaryl, substituted heteroaryl, heterocycle and substituted heterocycle; and
X³ is selected from a heteroatom, a substituted heteroatom, CH₂ and a substituted carbon atom.

17. The functional dye of claim 16, wherein formula (F1B) is of the structure:

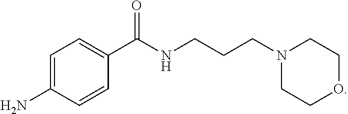 [SMILES: Nc1ccc(C(=O)NCCCN2CCOCC2)cc1]

18. The functional dye of claim 13, wherein fragment (F1) is of the formula (F1C): 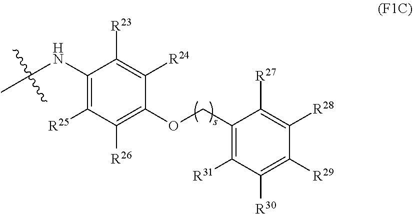
wherein:
s is an integer from 1 to 20; and
R²³ to R³¹ are each independently selected from H, alkyl, substituted alkyl, alkoxy, substituted alkoxy, halogen, CF₃, sulfonate, amino, substituted amino, amide, aryl, substituted aryl, heterocycle, substituted heterocycle, heterocycle and substituted heterocycle.

19. The functional dye of claim 18, wherein the formula (F1C) is of the structure:

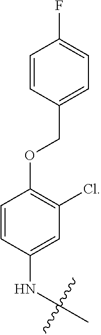 [SMILES: *Nc1ccc(OCc2ccc(F)cc2)c(Cl)c1]

20. The functional dye of claim 13, wherein the formula (F1) is of the structure:

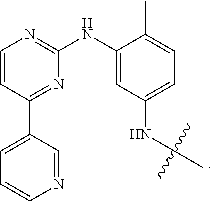 [SMILES: *Nc1ccc(C)c(Nc2nccc(-c3cccnc3)n2)c1]

21. The functional dye of any one of claims 1 to 20, wherein the near infrared fluorescent dye is a cyanine (Cy) dye.

22. The functional dye of claim 21, wherein the cyanine dye is of the formula (A): 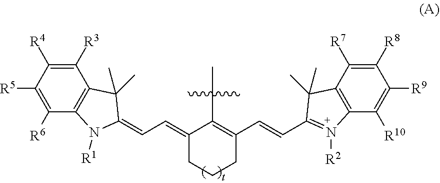
wherein:
R¹ and R² are each independently selected from H, alkyl, and (CH₂)ₙR¹², wherein n is an integer from 1 to 20 and R¹² is selected from the group consisting of H, sulfonate, carboxylic acid, amine, quaternary ammonium cation, phosphate, ester, halogen, azide, cyano, alkyne, and heterocycle;
R³ and R⁴, are each independently selected from H, alkyl, substituted alkyl, alkoxy, substituted alkoxy, halogen, CF₃, carboxy, phosphonate, carboxylate, cyano, hydroxyl, carboxyamide, sulfonamide, sulfonate, amino, substituted amino, amide, aryl, substituted aryl, heteroaryl, substituted heteroaryl, heterocycle and substituted heterocycle;
or R³ and R⁴ together with the carbon to which they are attached form a group selected from aryl, substituted aryl, heteroaryl, substituted heteroaryl, heterocycle, substituted heterocycle, cycloalkyl and substituted cycloalkyl;
R⁷ and R⁸ are each independently selected from H, alkyl, substituted alkyl, alkoxy, substituted alkoxy, halogen, CF₃, carboxy, phosphonate, carboxylate, cyano, hydroxyl, carboxyamide, sulfonamide, sulfonate, amino, substituted amino, amide, aryl, substituted aryl, heteroaryl, substituted heteroaryl, heterocycle and substituted heterocycle;
or R⁷ and R⁸ together with the carbon to which they are attached form a group selected from aryl, substituted aryl, heteroaryl, substituted heteroaryl, heterocycle, substituted heterocycle, cycloalkyl and substituted cycloalkyl;
R⁵, R⁶, R⁹ and R¹⁰ are each independently selected from H, alkyl, substituted alkyl, alkoxy, substituted alkoxy, halogen, CF₃, carboxy, phosphonate, carboxylate, cyano, hydroxyl, carboxyamide, sulfonamide, sulfonate, amino, substituted amino, amide, aryl, substituted aryl, heteroaryl, substituted heteroaryl, heterocycle and substituted heterocycle; and
t is an integer from 0 to 1.

23. The functional dye of claim 22, wherein the cyanine dye is substituted with at least one iodide.

24. The functional dye of claim 22 or 23, wherein the cyanine dye is substituted with at least one isotopic label.

25. The functional dye of claim 24, wherein the isotopic label is selected from ²H, ¹²⁰I, ¹²³I, ¹²⁴I, ¹²⁵I, ¹³¹I, ²⁰⁹As, ²¹⁰As, ²¹¹As, ³⁵S, ^{U}C, ¹³C, ¹⁴C, ³²P, ¹⁵N, ¹³N, ¹¹⁰In, ¹¹¹In, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹⁵⁴Gd, ¹⁵⁵Gd, ¹⁵⁵Gd, ¹⁵⁷Gd, ¹⁵⁸Gd, ¹⁵O, ¹⁸⁶Re, ¹⁸⁸Re, ⁵¹M, ^{52m}Mn, ⁵⁵Co, ⁷²As, ⁷⁵Br, ⁷⁶Br, ^{82m}Rb and ⁸³Sr.

26. The functional dye of claim 22, wherein the cyanine dye is selected from:

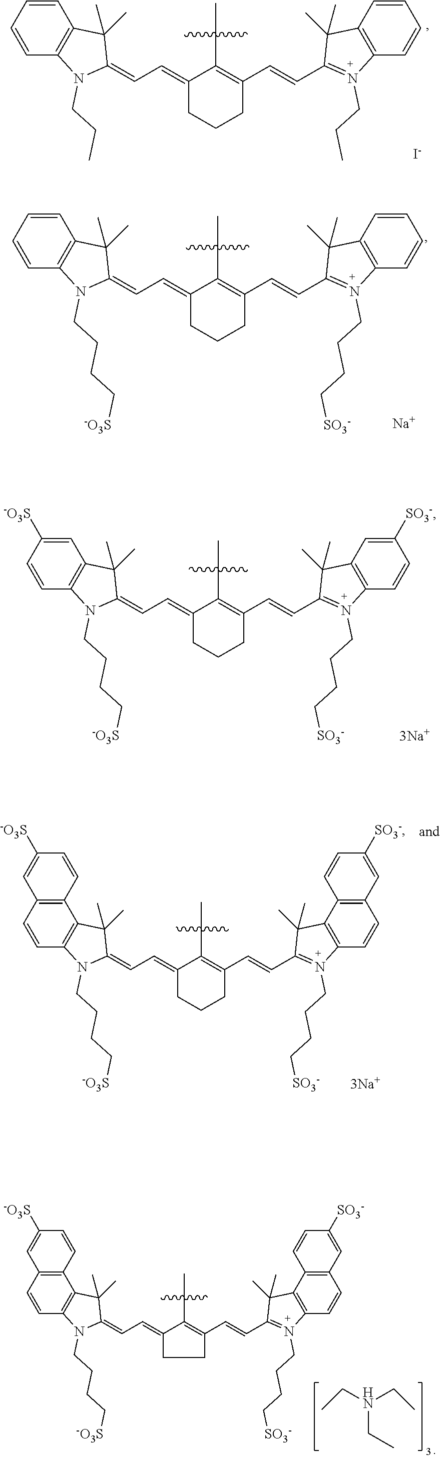

27. The functional dye of any one of claims 1 to 20, wherein the near infrared fluorescent dye is fluorescein or a derivative thereof.

28. The functional dye of claim 27, wherein the fluorescein derivative is fluorescein isothiocyanate (FITC).

29. The functional dye of any one of claims 1 to 20, wherein the near infrared fluorescent dye is rhodamine isothiocyanate (RITC).

30. The functional dye of any one of claims 1 to 3, wherein the functional dye is of the formula (A1): 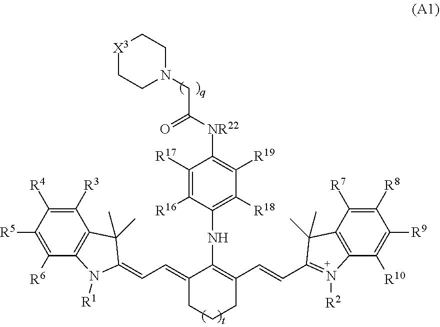
wherein:
R¹ and R² are each independently selected from H, alkyl, and (CH₂)ₙR¹², wherein n is an integer from 1 to 20 and R¹² is selected from the group consisting of H, sulfonate, carboxylic acid, amine, quaternary ammonium cation, phosphate, ester, halogen, azide, cyano, alkyne, and heterocycle;
R³ and R⁴, are each independently selected from H, alkyl, substituted alkyl, alkoxy, substituted alkoxy, halogen, CF₃, carboxy, phosphonate, carboxylate, cyano, hydroxyl, carboxyamide, sulfonamide, sulfonate, amino, substituted amino, amide, aryl, substituted aryl, heteroaryl, substituted heteroaryl, heterocycle and substituted heterocycle;
or R³ and R⁴ together with the carbon to which they are attached form a group selected from aryl, substituted aryl, heteroaryl, substituted heteroaryl, heterocycle, substituted heterocycle, cycloalkyl and substituted cycloalkyl;
R⁷ and R⁸ are each independently selected from H, alkyl, substituted alkyl, alkoxy, substituted alkoxy, halogen, CF₃, carboxy, phosphonate, carboxylate, cyano, hydroxyl, carboxyamide, sulfonamide, sulfonate, amino, substituted amino, amide, aryl, substituted aryl, heteroaryl, substituted heteroaryl, heterocycle and substituted heterocycle;
or R⁷ and R⁸ together with the carbon to which they are attached form a group selected from aryl, substituted aryl, heteroaryl, substituted heteroaryl, heterocycle, substituted heterocycle, cycloalkyl and substituted cycloalkyl; and
R⁵, R⁶, R⁹ and R¹⁰ are each independently selected from H, alkyl, substituted alkyl, alkoxy, substituted alkoxy, halogen, CF₃, carboxy, phosphonate, carboxylate, cyano, hydroxyl, carboxyamide, sulfonamide, sulfonate, amino, substituted amino, amide, aryl, substituted aryl, heteroaryl, substituted heteroaryl, heterocycle and substituted heterocycle;
T is an integer from 0 to 1;
q is an integer from 1 to 20;
R²² is selected from H, alkyl, substituted alkyl, aryl, substituted aryl, heterocycle, substituted heterocycle, heteroaryl, substituted heteroaryl;
R¹⁶, R¹⁷, R¹⁸ and R¹⁹ are each independently selected from H, alkyl, substituted alkyl, alkoxy, substituted alkoxy, halogen, CF₃, sulfonate, amino, substituted amino, amide, aryl, substituted aryl, heteroaryl, substituted heteroaryl, heterocycle and substituted heterocycle; and
X³ is selected from a heteroatom, a substituted heteroatom, CH₂ and a substituted carbon atom.

31. The functional dye of claim 30, selected from the following structures:

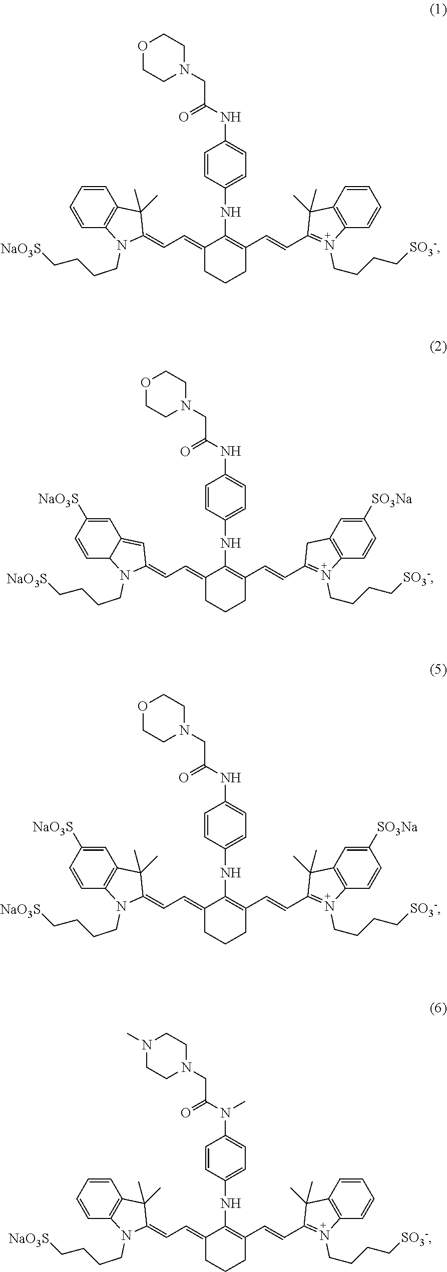

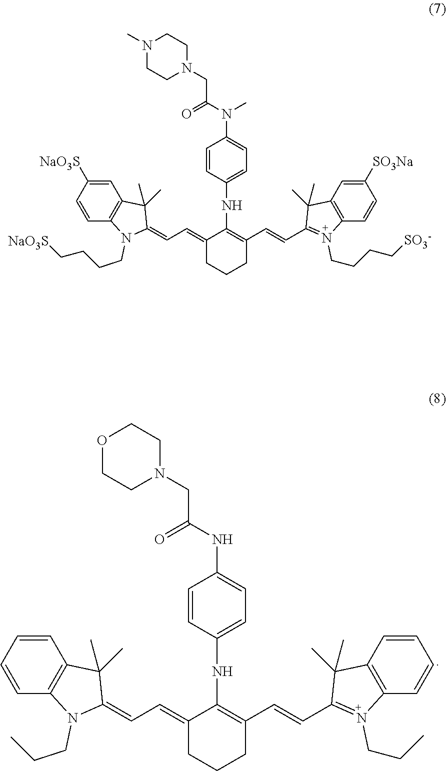

32. The functional dye of any one of claims 1 to 3, wherein the functional dye is of the formula (A1a): 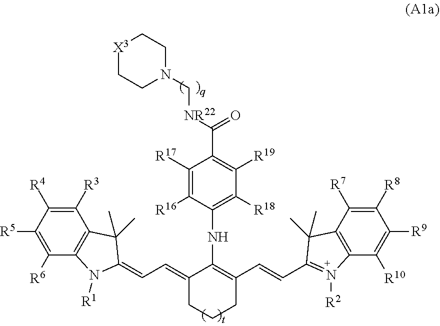
wherein:
R¹ and R² are each independently selected from H, alkyl, and (CH₂)ₙR¹², wherein n is an integer from 1 to 20 and R¹² is selected from the group consisting of H, sulfonate, carboxylic acid, amine, quaternary ammonium cation, phosphate, ester, halogen, azide, cyano, alkyne, and heterocycle;
R³ and R⁴, are each independently selected from H, alkyl, substituted alkyl, alkoxy, substituted alkoxy, halogen, CF₃, carboxy, phosphonate, carboxylate, cyano, hydroxyl, carboxyamide, sulfonamide, sulfonate, amino, substituted amino, amide, aryl, substituted aryl, heteroaryl, substituted heteroaryl, heterocycle and substituted heterocycle;
or R³ and R⁴ together with the carbon to which they are attached form a group selected from aryl, substituted aryl, heteroaryl, substituted heteroaryl, heterocycle, substituted heterocycle, cycloalkyl and substituted cycloalkyl;
R⁷ and R⁸ are each independently selected from H, alkyl, substituted alkyl, alkoxy, substituted alkoxy, halogen, CF₃, carboxy, phosphonate, carboxylate, cyano, hydroxyl, carboxyamide, sulfonamide, sulfonate, amino, substituted amino, amide, aryl, substituted aryl, heteroaryl, substituted heteroaryl, heterocycle and substituted heterocycle;
or R⁷ and R⁸ together with the carbon to which they are attached form a group selected from aryl, substituted aryl, heteroaryl, substituted heteroaryl, heterocycle, substituted heterocycle, cycloalkyl and substituted cycloalkyl; and
R⁵, R⁶, R⁹ and R¹⁰ are each independently selected from H, alkyl, substituted alkyl, alkoxy, substituted alkoxy, halogen, CF₃, carboxy, phosphonate, carboxylate, cyano, hydroxyl, carboxyamide, sulfonamide, sulfonate, amino, substituted amino, amide, aryl, substituted aryl, heteroaryl, substituted heteroaryl, heterocycle and substituted heterocycle;
T is an integer from 0 to 1;
q is an integer from 1 to 20;
R²² is selected from H, alkyl, substituted alkyl, aryl, substituted aryl, heterocycle, substituted heterocycle, heteroaryl, substituted heteroaryl;
R¹⁶, R¹⁷, R¹⁸ and R¹⁹ are each independently selected from H, alkyl, substituted alkyl, alkoxy, substituted alkoxy, halogen, CF₃, sulfonate, amino, substituted amino, amide, aryl, substituted aryl, heteroaryl, substituted heteroaryl, heterocycle and substituted heterocycle; and
X³ is selected from a heteroatom, a substituted heteroatom, CH₂ and a substituted carbon atom.

33. The functional dye of claim 32, of structure (10):

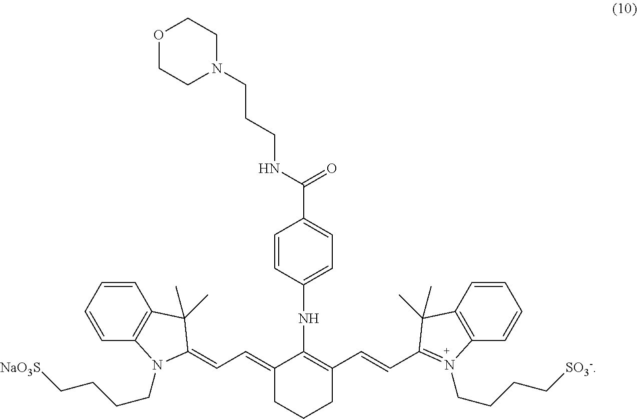

34. The functional dye of any one of claims 1 to 3, wherein the functional dye is of the formula (A2): 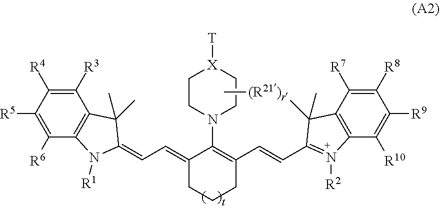
wherein:
R¹ and R² are each independently selected from H, alkyl, and (CH₂)ₙR¹², wherein n is an integer from 1 to 20 and R¹² is selected from the group consisting of H, sulfonate, carboxylic acid, amine, quaternary ammonium cation, phosphate, ester, halogen, azide, cyano, alkyne, and heterocycle;
R³ and R⁴, are each independently selected from H, alkyl, substituted alkyl, alkoxy, substituted alkoxy, halogen, CF₃, carboxy, phosphonate, carboxylate, cyano, hydroxyl, carboxyamide, sulfonamide, sulfonate, amino, substituted amino, amide, aryl, substituted aryl, heteroaryl, substituted heteroaryl, heterocycle and substituted heterocycle;
or R³ and R⁴ together with the carbon to which they are attached form a group selected from aryl, substituted aryl, heteroaryl, substituted heteroaryl, heterocycle, substituted heterocycle, cycloalkyl and substituted cycloalkyl;
R⁷ and R⁸ are each independently selected from H, alkyl, substituted alkyl, alkoxy, substituted alkoxy, halogen, CF₃, carboxy, phosphonate, carboxylate, cyano, hydroxyl, carboxyamide, sulfonamide, sulfonate, amino, substituted amino, amide, aryl, substituted aryl, heteroaryl, substituted heteroaryl, heterocycle and substituted heterocycle;
or R⁷ and R⁸ together with the carbon to which they are attached form a group selected from aryl, substituted aryl, heteroaryl, substituted heteroaryl, heterocycle, substituted heterocycle, cycloalkyl and substituted cycloalkyl; and
R⁵, R⁶, R⁹ and R¹⁰ are each independently selected from H, alkyl, substituted alkyl, alkoxy, substituted alkoxy, halogen, CF₃, carboxy, phosphonate, carboxylate, cyano, hydroxyl, carboxyamide, sulfonamide, sulfonate, amino, substituted amino, amide, aryl, substituted aryl, heteroaryl, substituted heteroaryl, heterocycle and substituted heterocycle;
t is an integer from 0 to 1;
X is selected from a N, CH and CR³², wherein R³² is selected from alkyl, substituted alkyl, hydroxy, alkoxy, substituted alkoxy, halogen, CF₃, sulfonate, amino, substituted amino, amide, aryl, substituted aryl, heteroaryl, substituted heteroaryl, heterocycle and substituted heterocycle;
T is a protein targeting moiety comprising a fragment of a known drug that targets a protein selected from the group consisting of (non)-receptor kinases, immune checkpoint proteins, G-protein-coupled receptors (GPCRs), influx transporters, efflux transporters, ion channels, human leukocyte antigens (HLA), proteases, caspases and nuclear receptors from a small molecule kinase inhibitor or a fragment of a small molecule modulator of GPCRs;
R^{21′} are each independently selected from alkyl, substituted alkyl, alkoxy, substituted alkoxy, halogen, CF₃, sulfonate, amino, substituted amino, amide, aryl, substituted aryl, heteroaryl, substituted heteroaryl, heterocycle and substituted heterocycle; and
r′ is an integer from 0 to 8.

35. The functional dye of claim 34, wherein T is a fragment of imatinib, bosutinib, nintedanib, ponatinib, brigatinib, dasatinib or palbociclib.

36. The functional dye of claim 34, wherein T is a fragment of sildenafil or eszopiclone.

37. The functional dye of claim 34, wherein T is selected from:

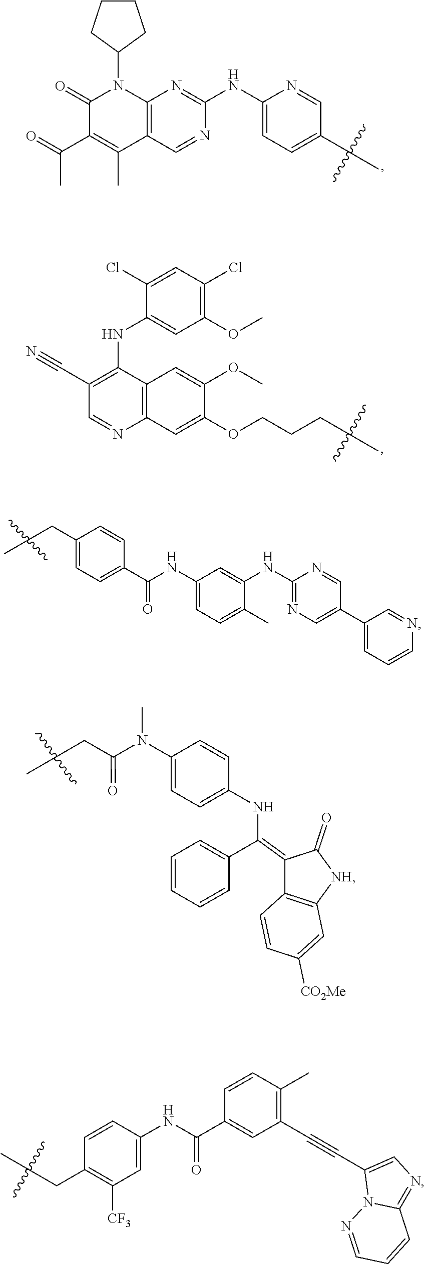 [SMILES: *c1ccc(Nc2ncc3c(C)c(C(C)=O)c(=O)n(C4CCCC4)c3n2)nc1 ; *CCCOc1cc2ncc(C#N)c(Nc3cc(OC)c(Cl)cc3Cl)c2cc1OC ; *Cc1ccc(C(=O)Nc2ccc(C)c(Nc3ncc(-c4cccnc4)cn3)c2)cc1 ; *CC(=O)N(C)c1ccc(N/C(=C2\C(=O)Nc3cc(C(=O)OC)ccc32)c2ccccc2)cc1 ; *Cc1ccc(NC(=O)c2ccc(C)c(C#Cc3cnc4cccnn34)c2)cc1C(F)(F)F]

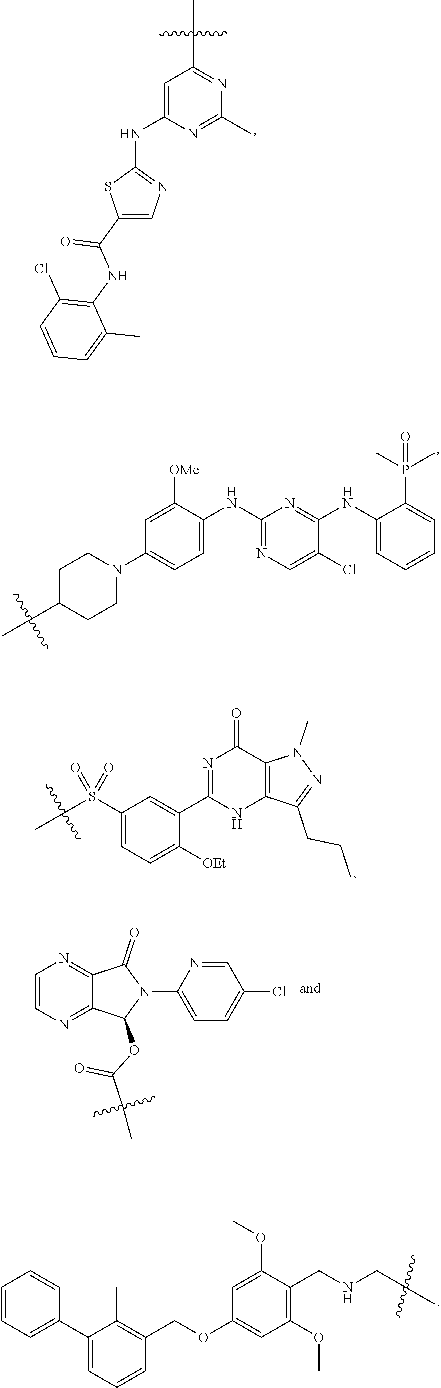 [SMILES: *c1cc(Nc2ncc(C(=O)Nc3c(C)cccc3Cl)s2)nc(C)n1 ; *C1CCN(c2ccc(Nc3ncc(Cl)c(Nc4ccccc4P(C)(C)=O)n3)c(OC)c2)CC1 ; *S(=O)(=O)c1ccc(OCC)c(-c2nc(=O)c3c([nH]2)c(CCC)nn3C)c1 ; *C(=O)O[C@H]1c2nccnc2C(=O)N1c1ccc(Cl)cn1 ; *CNCc1c(OC)cc(OCc2cccc(-c3ccccc3)c2C)cc1OC]

38. A pharmaceutical composition, comprising:
a functional dye of any one of claims 1-37; and
a pharmaceutically acceptable excipient.

39. A pharmaceutical composition for use as an oral contrast agent, comprising:
a functional dye of any one of claims 1-37; and
a pharmaceutically acceptable excipient.

40. The pharmaceutical composition of claim 38 or 39, further comprising one or more agents selected from PEG and sodium bicarbonate.

41. The pharmaceutical composition of claim 40, wherein the PEG is PEG3350.

42. A method of imaging a tumor, the method comprising:
administering a pharmaceutical composition according to any one of claims 38 to 41 to an individual;
detecting the presence of the functional dye;
wherein increased concentration of the dye is indicative of a lesion.

43. The method of claim 42, wherein the functional dye is detected with a fluorescence endoscopic camera or a fluorescence bronchoscopic camera.

44. The method of claim 42, wherein the functional dye is detected with a fluorescence capsule endoscopic camera, cystoscope or a laparoscope.

45. The method of claim 43 or 44 wherein the camera is a combined white light near-infrared (WL/NIRF) camera.

46. The method of any one of claims 42 to 45, wherein the lesion is a cancerous or precancerous lesion.

47. The method of claim 46, wherein the lesion is found in one or more locations selected from the gastrointestinal tract, breast, brain, prostate, pancreas, skin, bladder, head, neck and thyroid.

48. The method of any one of claims 42 to 27, wherein administration of the functional dye is oral.

49. The method of any one of claims 42 to 48, wherein detection of the dye is used to guide biopsy or surgical resection of the lesion.

50. A kit comprising:
a functional dye of any one of claims 1-37; and
a device for detecting the presence of the functional dye.

51. The kit of claim 50, wherein the device for detection of the functional dye is selected from a capsule endoscope, a portable cytometer, a smartphone add on device and a wearable cytometer.

52. The kit of claim 52, wherein the smartphone add on device is a combined NIRF/WL camera unit.
